(19) United States
(12) Patent Application Publication
(10) Pub. No.: US 2005/0245534 A1
(43) Pub. Date: Nov. 3, 2005

(54) Inhibitors of the 11-beta-hydroxysteroid dehydrogenase Type 1 enzyme

(76) Inventors: James T. Link, Evanston, IL (US); Marina A. Pliushchev, Vernon Hills, IL (US); Jeffrey J. Rohde, Evanston, IL (US); Dariusz Wodka, Waukegan, IL (US); Jyoti R. Patel, Libertyville, IL (US); Qi Shuai, Gurnee, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/965,591

(22) Filed: Oct. 14, 2004

Related U.S. Application Data
(63) Continuation-in-part of application No. 10/834,459, filed on Apr. 29, 2004.

(51) Int. Cl. A61K 31/495; A61K 31/496; A61K 31/445; C07D 45/02; C07D 211/06

(52) U.S. Cl. 514/252.12; 514/254.1; 514/319; 544/380; 544/379; 546/203

(57) ABSTRACT

The present invention relates to compounds which are inhibitors of the 11-beta-hydroxysteroid dehydrogenase Type 1 enzyme. The present invention further relates to the use of inhibitors of 11-beta-hydroxysteroid dehydrogenase Type 1 enzyme for the treatment of non-insulin dependent type 2 diabetes, insulin resistance, obesity, lipid disorders, metabolic syndrome, and other diseases and conditions that are mediated by excessive glucocorticoid action.

This application is a continuation-in-part of U.S. patent application Ser. No. 10/834,459, filed Apr. 29, 2004, which is hereby incorporated by reference.

### FIELD OF INVENTION

The present invention relates to compounds which are inhibitors of the 11-beta-hydroxysteroid dehydrogenase Type 1 enzyme. The present invention further relates to the use of inhibitors of 11-beta-hydroxysteroid dehydrogenase Type 1 enzyme for the treatment of non-insulin dependent type 2 diabetes, insulin resistance, obesity, lipid disorders, metabolic syndrome, and other diseases and conditions that are mediated by excessive glucocorticoid action.

### BACKGROUND OF THE INVENTION

Insulin is a hormone which modulates glucose and lipid metabolism. Impaired action of insulin (i.e., insulin resistance) results in reduced insulin-induced glucose uptake, oxidation and storage, reduced insulin-dependent suppression of fatty acid release from adipose tissue (i.e., lipolysis), and reduced insulin-mediated suppression of hepatic glucose production and secretion. Insulin resistance frequently occurs in diseases that lead to increased and premature morbidity and mortality.

Diabetes mellitus is characterized by an elevation of plasma glucose levels (hyperglycemia) in the fasting state or after administration of glucose during a glucose tolerance test. While this disease may be caused by several underlying factors, it is generally grouped into two categories, Type 1 and Type 2 diabetes. Type 1 diabetes, also referred to as Insulin Dependent Diabetes Mellitus (“IDDM”), is caused by a reduction of production and secretion of insulin. In type 2 diabetes, also referred to as non-insulin dependent diabetes mellitus, or NIDDM, insulin resistance is a significant pathogenic factor in the development of hyperglycemia. Typically, the insulin levels in type 2 diabetes patients are elevated (i.e., hyperinsulinemia), but this compensatory increase is not sufficient to overcome the insulin resistance. Persistent or uncontrolled hyperglycemia in both type 1 and type 2 diabetes mellitus is associated with increased incidence of macrovascular and/or microvascular complications including atherosclerosis, coronary heart disease, peripheral vascular disease, stroke, nephropathy, neuropathy, and retinopathy.

Insulin resistance, even in the absence of profound hyperglycemia, is a component of the metabolic syndrome. Recently, diagnostic criteria for metabolic syndrome have been established. To qualify a patient as having metabolic syndrome, three out of the five following criteria must be met: elevated blood pressure above 130/85 mmHg, fasting blood glucose above 110 mg/dl, abdominal obesity above 40″ (men) or 35″ (women) waist circumference, and blood lipid changes as defined by an increase in triglycerides above 150 mg/dl or decreased HDL cholesterol below 40 mg/dl (men) or 50 mg/dl (women). It is currently estimated that 50 million adults, in the US alone, fulfill these criteria. That population, whether or not they develop overt diabetes mellitus, are at increased risk of developing the macrovascular and microvascular complications of type 2 diabetes listed above.

Available treatments for type 2 diabetes have recognized limitations. Diet and physical exercise can have profound beneficial effects in type 2 diabetes patients, but compliance is poor. Even in patients having good compliance, other forms of therapy may be required to further improve glucose and lipid metabolism.

One therapeutic strategy is to increase insulin levels to overcome insulin resistance. This may be achieved through direct injection of insulin or through stimulation of the endogenous insulin secretion in pancreatic beta cells. Sulfonylureas (e.g., tolbutamide and glipizide) or meglitinide are examples of drugs that stimulate insulin secretion (i.e., insulin secretagogues) thereby increasing circulating insulin concentrations high enough to stimulate insulin-resistant tissue. However, insulin and insulin secretagogues may lead to dangerously low glucose concentrations (i.e., hypoglycemia). In addition, insulin secretagogues frequently lose therapeutic potency over time.

Two biguanides, metformin and phenformin, may improve insulin sensitivity and glucose metabolism in diabetic patients. However, the mechanism of action is not well understood. Both compounds may lead to lactic acidosis and gastrointestinal side effects (e.g., nausea or diarrhea).

Alpha-glucosidase inhibitors (e.g., acarbose) may delay carbohydrate absorption from the gut after meals, which may in turn lower blood glucose levels, particularly in the postprandial period. Like biguanides, these compounds may also cause gastrointestinal side effects.

Glitazones (i.e., 5-benzylthiazolidine-2,4-diones) are a newer class of compounds used in the treatment of type 2 diabetes. These agents may reduce insulin resistance in multiple tissues, thus lowering blood glucose. The risk of hypoglycemia may also be avoided. Glitazones modify the activity of the Peroxisome Proliferator Activated Receptor (“PPAR”) gamma subtype. PPAR is currently believed to be the primary therapeutic target for the main mechanism of action for the beneficial effects of these compounds. Other modulators of the PPAR family of proteins are currently in development for the treatment of type 2 diabetes and/or dyslipidemia. Marketed glitazones suffer from side effects including bodyweight gain and peripheral edema.

Additional treatments to normalize blood glucose levels in patients with diabetes mellitus are needed. Other therapeutic strategies are being explored. For example, research is being conducted concerning Glucagon-Like Peptide 1 (“GLP-1”) analogues and inhibitors of Dipeptidyl Peptidase IV (“DPP-IV”) that increase insulin secretion. Other examples include: Inhibitors of key enzymes involved in the hepatic glucose production and secretion (e.g., fructose-1,6-bisphosphatase inhibitors), and direct modulation of enzymes involved in insulin signaling (e.g., Protein Tyrosine Phosphatase-1B, or “PTP-1B”).

Another method of treating or prophylactically treating diabetes mellitus includes using inhibitors of 11-β-hydroxysteroid dehydrogenase Type 1 (11β-HSD1). Such methods are discussed in J. R. Seckl et al., Endocrinology, 142: 1371-1376, 2001, and references cited therein. Glucocorticoids are steroid hormones that are potent regulators of glucose and lipid metabolism. Excessive glucocorticoid action may lead to insulin resistance, type 2 diabetes, dyslipidemia, increased abdominal obesity, and hypertension. Glucocorticoids circulate in the blood in an active form (i.e., cortisol in humans) and an inactive form (i.e., cortisone in humans). 11β-HSD1, which is highly expressed in liver and adipose tissue, converts cortisone to cortisol leading to higher local concentration of cortisol. Inhibition of 11β-HSD 1 prevents or decreases the tissue specific amplification of glucocorticoid action thus imparting beneficial effects on blood pressure and glucose- and lipid-metabolism.

Thus, inhibiting 11β-HSD1 benefits patients suffering from non-insulin dependent type 2 diabetes, insulin resistance, obesity, lipid disorders, metabolic syndrome, and other diseases and conditions mediated by excessive glucocorticoid action.

### SUMMARY OF THE INVENTION

One aspect of the present invention is directed toward a compound of formula (I) 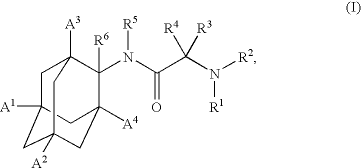
wherein
A¹, A², A³, and A⁴ are each individual members selected from the group consisting of hydrogen, alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸R⁹)]ₙ, —C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and, —C(R²³R²⁴)—N(R²⁵R²⁶);
n is 0 or 1;
p is 0 or 1;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, R³, R⁴ and the intervening carbon atoms form a non-aromatic heterocycle, R², R³ and the intervening carbon and nitrogen atoms form a non-aromatic heterocycle;
R⁵ is a member selected from the group consisting of hydrogen, alkyl, carboxyalkyl, carboxycycloalkyl, cycloalkyl, aryl, arylalkyl, aryloxyalkyl, heterocycle, heterocyclealkyl, and, heterocycleoxyalkyl;
R⁶ is a member selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, and, heterocycleoxyalkyl;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and, heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalky, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and, —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and, —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and, heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and, heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and, heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle;
provided that, if R⁶ is hydrogen; then at least one of A¹, A², A³ and A⁴ is not hydrogen.

A further aspect of the present invention encompasses the use of the compounds of formula (I) for the treatment of disorders that are mediated by 11-beta-hydroxysteroid dehydrogenase Type 1 enzyme, such as non-insulin dependent type 2 diabetes, insulin resistance, obesity, lipid disorders, metabolic syndrome, and other diseases and conditions that are mediated by excessive glucocorticoid action.

According to still another aspect, the present invention is directed to a pharmaceutical composition comprising a therapeutically effective amount of a compound of formula (I) in combination with a pharmaceutically suitable carrier.

### DETAILED DESCRIPTION OF THE INVENTION

All patents, patent applications, and literature references cited in the specification are herein incorporated by reference in their entirety.

One aspect of the present invention is directed toward a compound of formula (I) 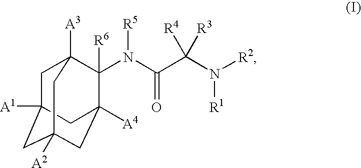
wherein
A¹, A², A³, and A⁴ are each individual members selected from the group consisting of hydrogen, alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸R⁹)]ₙ—C(O)—R¹⁰, —O—[C(R¹¹R¹²)]β—C(O)—R¹³, —OR¹⁴, —N(R¹⁵R¹⁶), —CO₂R¹⁷, —C(O)—N(R¹⁸R¹⁹), —C(R²⁰R²¹)—OR²² and, —C(R²³R²⁴)—N(R²¹R²⁶);
n is 0 or 1;
p is 0 or 1;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, R³, R⁴ and the intervening carbon atoms form a non-aromatic heterocycle, R², R³ and the intervening carbon and nitrogen atoms form a non-aromatic heterocycle;
R⁵ is a member selected from the group consisting of hydrogen, alkyl, carboxyalkyl, carboxycycloalkyl, cycloalkyl, aryl, arylalkyl, aryloxyalkyl, heterocycle, heterocyclealkyl, and, heterocycleoxyalkyl;
R⁶ is a member selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, and, heterocycleoxyalkyl;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and, heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalky, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and, —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and, —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and, heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and, heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and, heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle;
provided that, if R⁶ is hydrogen; then at least one of A¹, A², A³ and A⁴ is not hydrogen.

Another aspect of the present invention is directed toward a therapeutically suitable prodrug of a compound of formula (I).

Another aspect of the present invention is directed toward a therapeutically suitable salt of a compound of formula (I).

Another aspect of the present invention is directed toward a therapeutically suitable metabolite of a compound of formula (I).

Another aspect of the present invention is directed toward a compound of formula (II), 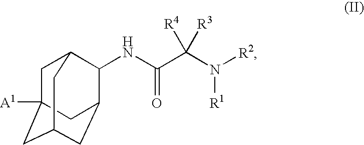
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹, —C(R²R²)—OR, and, —C(R²³R²⁴)—N(R²⁵R²⁶);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, R³, R⁴ and the intervening carbon atoms form a non-aromatic heterocycle, R², R³ and the intervening carbon and nitrogen atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and, heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalky, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and, —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and, —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and, heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and, heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and, heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle;
provided that, if R⁶ is hydrogen; then at least one of A¹, A², A³ and A⁴ is not hydrogen.

Another aspect of the present invention is directed toward a compound of formula (IIa), 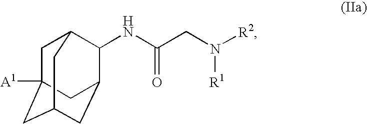
wherein
A¹ is a member selected from the group consisting of —OH, —CO₂H, carboxyalkyl, carboxycycloalky, and —C(O)—N(R¹⁸R¹⁹);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IIb), 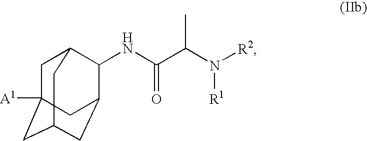
wherein
A¹ is a member selected from the group consisting of —OH, —CO₂H, carboxyalkyl, carboxycycloalkyl, and —C(O)—N(R¹⁸R¹⁹);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IIc), 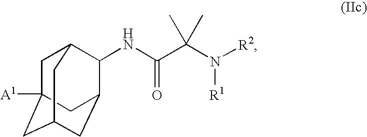
wherein
A¹ is a member selected from the group consisting of —OH, —CO₂H, carboxyalkyl, carboxycycloalkyl, and —C(O)—N(R¹⁸R¹⁹);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IIId), 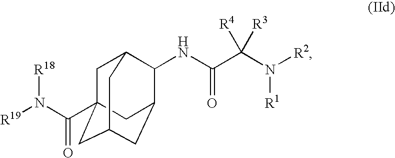
wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and the intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, R³, R⁴ and the intervening carbon atoms form a non-aromatic heterocycle, R², R³ and the intervening carbon and nitrogen atoms form a non-aromatic heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IIe), 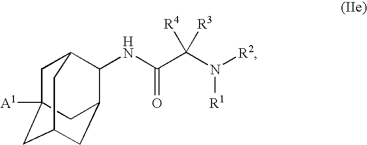
wherein
A¹ is a member selected from the group consisting of aryl and heterocycle;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, halo alkyl, heterocycle, heterocyclealkyl, heterocycle-hetero cycle, aryl-heterocycle, and, R¹, R² and the intervening atoms form a heterocycle; and,
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, R³, R⁴ and the intervening carbon atoms form a non-aromatic heterocycle, R², R³ and the intervening carbon and nitrogen atoms form a non-aromatic heterocycle.

Another aspect of the present invention is directed toward a therapeutically suitable prodrug of a compound of formula (II).

Another aspect of the present invention is directed toward a therapeutically suitable salt of a compound of formula (II).

Another aspect of the present invention is directed toward a therapeutically suitable metabolite of a compound of formula (II).

Another aspect of the present invention is directed toward a compound of formula (III), 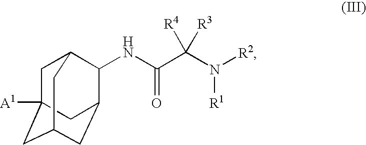
wherein
A¹ is a member selected from the group consisting of alkyl, alkyl-NH-alkyl, alkylcarbonyl, alkylsulfonyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹⁸R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and, —C(R²³R²⁴)—N(R²⁵R²⁶;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and aryl-heterocycle;
R³ and R⁴ are each a member independently selected from the group consisting of hydrogen, alkyl, carboxyalkyl, carboxycycloalkyl, cycloalkyl, aryl, and, heterocycle;
R⁷ is selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰;
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R^{27,} R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle.

Another aspect of the present invention is directed toward a compound of formula (IIIa), 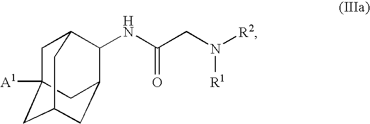
wherein
A¹ is a member selected from the group consisting of —OH, —CO₂H, carboxyalkyl, carboxycycloalky, and —C(O)—N(R¹⁸R¹⁹);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and aryl-heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IIIb), 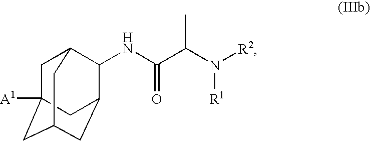
wherein
A¹ is a member selected from the group consisting of —OH, —CO₂H, carboxyalkyl, carboxycycloalkyl, and —C(O)—N(R¹⁸R¹⁹);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and aryl-heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IIIc), 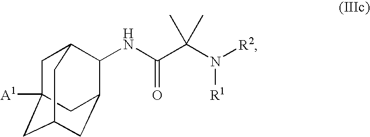
wherein
A¹ is a member selected from the group consisting of —OH, —CO₂H, carboxyalkyl, carboxycycloalkyl, and —C(O)—N(R¹⁸R¹⁹);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and aryl-heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IIId), 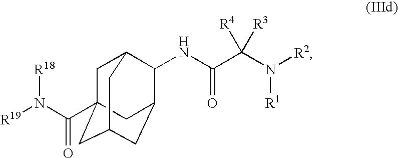
wherein
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and aryl-heterocycle;
R³ and R⁴ are each a member independently selected from the group consisting of hydrogen, alkyl, carboxyalkyl, carboxycycloalkyl, cycloalkyl, aryl, and heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R⁸, R⁹ and the intervening atoms form a non-aromatic heterocycle.

Another aspect of the present invention is directed toward a compound of formula (IIIe), 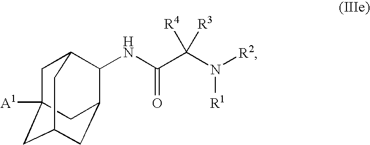
wherein
A¹ is a member selected from the group consisting of aryl and heterocycle;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle; and,
R³ and R⁴ are each a member independently selected from the group consisting of hydrogen, alkyl, carboxyalkyl, carboxycycloalkyl, cycloalkyl, aryl, and, heterocycle.

Another aspect of the present invention is directed toward a therapeutically suitable prodrug of a compound of formula (III).

Another aspect of the present invention is directed toward a therapeutically suitable salt of a compound of formula (III).

Another aspect of the present invention is directed toward a therapeutically suitable metabolite of a compound of formula (III).

Another aspect of the present invention is directed toward a compound of formula (IV), 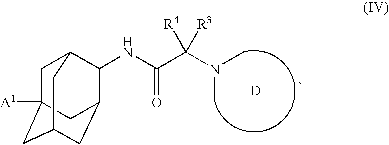
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ, —C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and, —C(R²³R²⁴)—N(R²⁵R²⁶);
D is a non-aromatic heterocycle;
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, and R³, R⁴ and the intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R^{27,} R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle.

Another aspect of the present invention is directed toward a compound of formula (IVa), 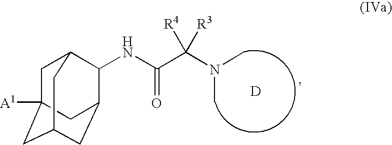
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ, —C(O)—R¹⁰, —O—[C(R¹¹R¹²)]ₚ—C(O)—R¹³, —OR¹⁴, —N(R¹⁵R⁶), —CO₂R¹⁷, —C(O)—N(R¹⁸R¹⁹), —C(R²⁰R²¹)—OR²², and —C(R²³R²⁴)—N(R²⁵R²⁶);
D is a non-aromatic heterocycle;
R³ and R⁴ are each a member independently selected from the group consisting of hydrogen, alkyl, carboxyalkyl, carboxycycloalkyl, cycloalkyl, aryl, and, heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R⁹, R³⁰ and the intervening atoms form a non-aromatic heterocycle.

Another aspect of the present invention is directed toward a compound of formula (IVb), 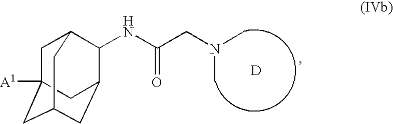
wherein
A¹ is a member selected from the group consisting of —OH, —CO₂H, carboxyalkyl, carboxycycloalkyl, and —C(O)—N(R¹⁸R¹⁹);
D is a non-aromatic heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IVc), 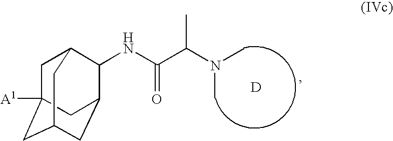
wherein
A¹ is a member selected from the group consisting of —OH, —CO₂H, carboxyalkyl, carboxycycloalkyl, and —C(O)—N(R¹⁸R¹⁹);
D is a non-aromatic heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IVd), 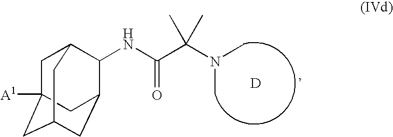
wherein
A¹ is a member selected from the group consisting of —OH, —CO₂H, carboxyalkyl, carboxycycloalkyl, and —C(O)—N(R¹⁸R¹⁹);
D is a non-aromatic heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a therapeutically suitable prodrug of a compound of formula (IV).

Another aspect of the present invention is directed toward a therapeutically suitable salt of a compound of formula (IV).

Another aspect of the present invention is directed toward a therapeutically suitable metabolite of a compound of formula (IV).

Another aspect of the present invention is directed toward a compound of formula (V), 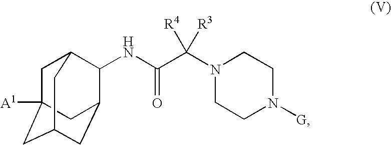
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶);
G is a member selected from the group consisting of aryl and heterocycle;
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, and R³, R⁴ and the intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R^{27,} R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle.

Another aspect of the present invention is directed toward a compound of formula (Va), 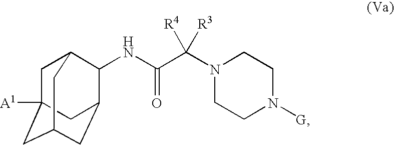
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶);
G is a member selected from the group consisting of aryl and heterocycle;
R³ and R⁴ are each a member independently selected from the group consisting of hydrogen, alkyl, carboxyalkyl, carboxycycloalkyl, cycloalkyl, aryl, and, heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R^{27,} R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle.

Another aspect of the present invention is directed toward a compound of formula (Vb), 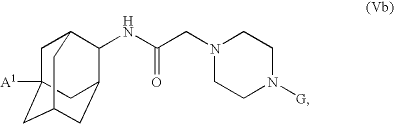
wherein
A¹ is a member selected from the group consisting of —OH, —CO₂H, carboxyalkyl, carboxycycloalkyl, and —C(O)—N(R¹⁸R¹⁹);
G is a member selected from the group consisting of aryl and heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (Vc), 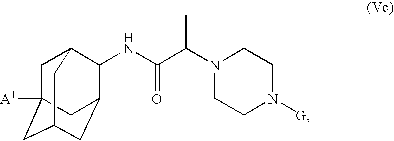
wherein
A¹ is a member selected from the group consisting of —OH, —CO₂H, carboxyalkyl, carboxycycloalkyl, and —C(O)—N(R¹⁸R¹⁹);
G is a member selected from the group consisting of aryl and heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (Vd), 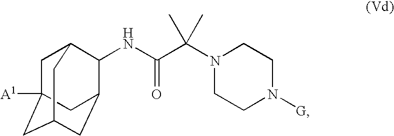
wherein
A¹ is a member selected from the group consisting of —OH, —CO₂H, carboxyalkyl, carboxycycloalkyl, and —C(O)—N(R¹⁸R¹⁹);
G is a member selected from the group consisting of aryl and heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (Ve), 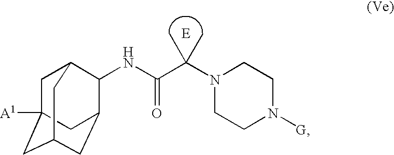
wherein
A¹ is a member selected from the group consisting of —OH, —CO₂H, carboxyalkyl, carboxycycloalkyl, and —C(O)—N(R¹⁸R¹⁹);
G is a member selected from the group consisting of aryl and heterocycle;
E is a member selected from the group consisting of cycloalkyl and non-aromatic heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a therapeutically suitable prodrug of a compound of formula (V).

Another aspect of the present invention is directed toward a therapeutically suitable salt of a compound of formula (V).

Another aspect of the present invention is directed toward a therapeutically suitable metabolite of a compound of formula (V).

Another aspect of the present invention is directed toward a compound of formula (VI), 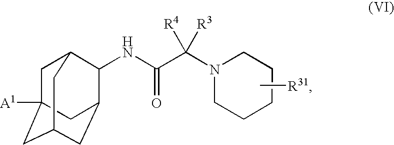
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶);
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, and R³, R⁴ and the intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R^{27,} R²⁸ and the intervening atoms form a non-aromatic heterocycle;
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIa), 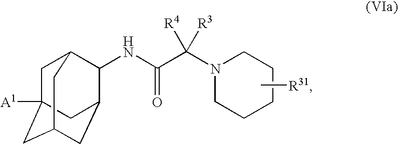
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶); and
R³ and R⁴ are each a member independently selected from the group consisting of hydrogen, alkyl, carboxyalkyl, carboxycycloalkyl, cycloalkyl, haloalkyl, aryl, and, heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹ and R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R^{27,} R²⁸ and the intervening atoms form a non-aromatic heterocycle;
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIb), 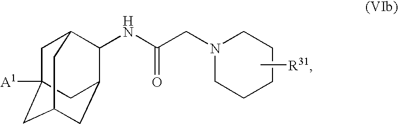
wherein
A¹ is a member selected from the group consisting of —OH, —CO₂H, carboxyalkyl, carboxycycloalkyl, and, —C(O)—N(R¹⁸R¹⁹);
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIc), 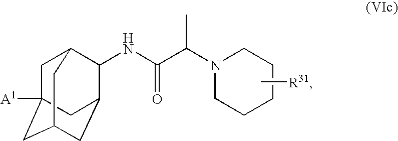
wherein
A¹ is a member selected from the group consisting of —OH, —CO₂H, carboxyalkyl, carboxycycloalkyl, and —C(O)—N(R¹⁸R¹⁹);
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VId), 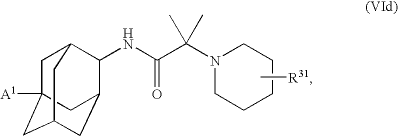
wherein
A¹ is a member selected from the group consisting of —OH, —CO₂H, carboxyalkyl, carboxycycloalkyl, and —C(O)—N(R¹⁸R¹⁹);
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIe), 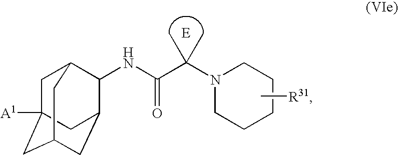
wherein
A¹ is a member selected from the group consisting of —OH, —CO₂H, carboxyalkyl, carboxycycloalkyl, and —C(O)—N(R¹⁸R¹⁹);
E is a member selected from the group consisting of cycloalkyl and non-aromatic heterocycle;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a therapeutically suitable prodrug of a compound of formula (VI).

Another aspect of the present invention is directed toward a therapeutically suitable salt of a compound of formula (VI).

Another aspect of the present invention is directed toward a therapeutically suitable metabolite of a compound of formula (VI).

Another aspect of the present invention is directed toward a compound of formula (VII), 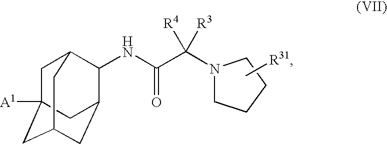
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶);
R³ and R⁴ are each a member independently selected from the group consisting of hydrogen, alkyl, carboxyalkyl, carboxycycloalkyl, cycloalkyl, haloalkyl, aryl, heterocycle, R³, R⁴ and the intervening atoms form a cycloalkyl, and R³, R⁴ and the intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and the intervening atoms form a non-aromatic heterocycle;
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIIa), 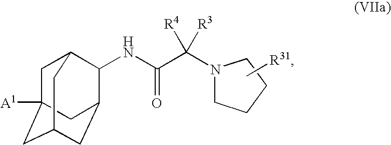
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶); and
R³ and R⁴ are each a member independently selected from the group consisting of hydrogen, alkyl, carboxyalkyl, carboxycycloalkyl, cycloalkyl, aryl, and, heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and the intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and the intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and the intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and the intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R^{27,} R²⁸ and the intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIIb), 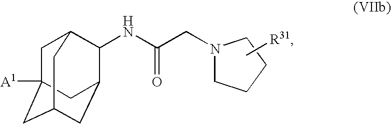
wherein
A¹ is a member selected from the group consisting of —OH, —CO₂H, carboxyalkyl, carboxycycloalkyl, and —C(O)—N(R¹⁸R¹⁹);
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIIc), 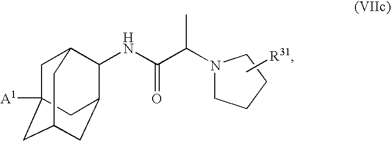
wherein
A¹ is a member selected from the group consisting of —OH, —CO₂H, carboxyalkyl, carboxycycloalkyl, and —C(O)—N(R¹⁸R¹⁹);
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIId), 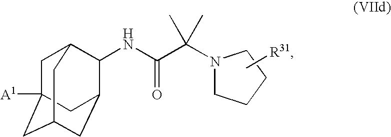
wherein
A¹ is a member selected from the group consisting of —OH, —CO₂H, carboxyalkyl, carboxycycloalkyl, and —C(O)—N(R¹⁸R¹⁹);
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (VIIe), 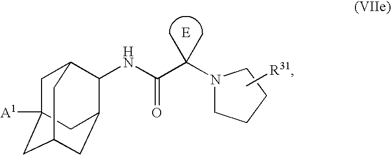
wherein
A¹ is a member selected from the group consisting of —OH, —CO₂H, carboxyalkyl, carboxycycloalkyl, and —C(O)—N(R¹⁸R¹⁹);
E is a member selected from the group consisting of cycloalkyl and non-aromatic heterocycle;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a therapeutically suitable prodrug of a compound of formula (VII).

Another aspect of the present invention is directed toward a therapeutically suitable salt of a compound of formula (VII).

Another aspect of the present invention is directed toward a therapeutically suitable metabolite of a compound of formula (VII).

Another aspect of the present invention is directed toward a compound of formula (VIII), 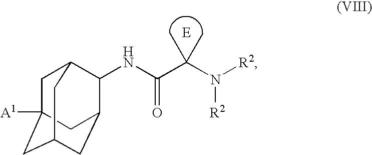
wherein
A¹ is a member selected from the group consisting of —OH, —CO₂H, carboxyalkyl, carboxycycloalkyl, and —C(O)—N(R¹⁸R¹⁹);
E is a member selected from the group consisting of cycloalkyl and non-aromatic heterocycle;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a therapeutically suitable prodrug of a compound of formula (VIII).

Another aspect of the present invention is directed toward a therapeutically suitable salt of a compound of formula (VIII).

Another aspect of the present invention is directed toward a therapeutically suitable metabolite of a compound of formula (VIII).

Another aspect of the present invention is directed toward a compound of formula (IX), 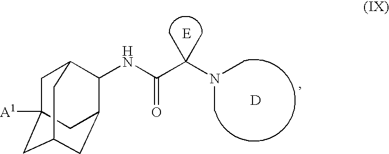
wherein
A¹ is a member selected from the group consisting of —OH, —CO₂H, carboxyalkyl, carboxycycloalkyl, and —C(O)—N(R¹⁸R¹⁹);
D is a non-aromatic heterocycle;
E is a member selected from the group consisting of cycloalkyl and non-aromatic heterocycle; and
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IXa), 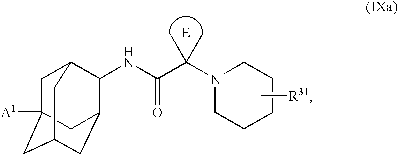
wherein
A¹ is a member selected from the group consisting of —OH, —CO₂H, carboxyalkyl, carboxycycloalkyl, and —C(O)—N(R¹⁸R¹⁹);
E is a member selected from the group consisting of cycloalkyl and non-aromatic heterocycle;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the present invention is directed toward a compound of formula (IXb), 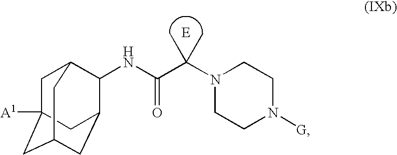
wherein
A¹ is a member selected from the group consisting of —OH, —CO₂H, carboxyalkyl, carboxycycloalkyl, and —C(O)—N(R¹⁸R¹⁹);
G is a member selected from the group consisting of aryl and heterocycle; and
E is a member selected from the group consisting of cycloalkyl and non-aromatic heterocycle; and
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle.

Another aspect of the present invention is directed toward a compound of formula (IXc), 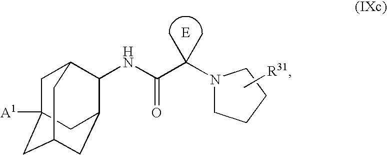
wherein
A¹ is a member selected from the group consisting of —OH, —CO₂H, carboxyalkyl, carboxycycloalkyl, and —C(O)—N(R¹⁸R¹⁹);
E is a member selected from the group consisting of cycloalkyl and non-aromatic heterocycle;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and the intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

Another aspect of the invention includes a method of inhibiting 11-beta-hydroxysteroid dehydrogenase Type I enzyme comprising administering to a mammal, a therapeutically effective amount of a compound of formula (I).

Another aspect of the invention includes a method of inhibiting 11-beta-hydroxysteroid dehydrogenase Type I enzyme comprising administering to a mammal, a therapeutically effective amount of a compound of formula (II).

Another aspect of the invention includes a method of inhibiting 11-beta-hydroxysteroid dehydrogenase Type I enzyme comprising administering to a mammal, a therapeutically effective amount of a compound of formula (III).

Another aspect of inhibiting 11-beta-hydroxysteroid dehydrogenase Type I enzyme comprising administering to a mammal, a therapeutically effective amount of a compound of formula (IV).

Another aspect of the invention includes a method of inhibiting 11-beta-hydroxysteroid dehydrogenase Type I enzyme comprising administering to a mammal, a therapeutically effective amount of a compound of formula (V).

Another aspect of the invention includes a method of inhibiting 11-beta-hydroxysteroid dehydrogenase Type I enzyme comprising administering to a mammal, a therapeutically effective amount of a compound of formula (VI).

Another aspect of the invention includes a method of inhibiting 11-beta-hydroxysteroid dehydrogenase Type I enzyme comprising administering to a mammal, a therapeutically effective amount of a compound of formula (VII).

Another aspect of the invention includes a method of inhibiting 11-beta-hydroxysteroid dehydrogenase Type I enzyme comprising administering to a mammal, a therapeutically effective amount of a compound of formula (VIII).

Another aspect of the invention includes a method of inhibiting 11-beta-hydroxysteroid dehydrogenase Type I enzyme comprising administering to a mammal, a therapeutically effective amount of a compound of formula (IX).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (I).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (II).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (III).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (IV).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (V).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (VI).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (VII).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (VIII).

Another aspect of the invention includes a method of treating or prophylactically treating disorders in a mammal by inhibiting 11-beta-hydroxysteroid dehydrogenase Type I enzyme comprising administering to a mammal, a therapeutically effective amount of a compound of formula (IX).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (I).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (II).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (III).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (IV).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (V).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (VI).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (VII).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (VIII).

Another aspect of the invention includes a method of treating or prophylactically treating non-insulin dependent type 2 diabetes in a mammal by inhibiting 11-beta-hydroxysteroid dehydrogenase Type I enzyme comprising administering to a mammal, a therapeutically effective amount of a compound of formula (IX).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (I).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (II).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (III).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (IV).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (V).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (VI).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (VII).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (VIII).

Another aspect of the invention includes a method of treating or prophylactically treating insulin resistance in a mammal by inhibiting 11-beta-hydroxysteroid dehydrogenase Type I enzyme comprising administering to a mammal, a therapeutically effective amount of a compound of formula (IX).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (I).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (II).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (III).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (IV).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (V).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (VI).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (VII).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (VIII).

Another aspect of the invention includes a method of treating or prophylactically treating obesity in a mammal by inhibiting 11-beta-hydroxysteroid dehydrogenase Type I enzyme comprising administering to a mammal, a therapeutically effective amount of a compound of formula (IX).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (I).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (II).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (III).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (IV).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (V).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (VI).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (VII).

Another aspect of the invention includes a method of treating or prophylactically treating lipid disorders in a mammal by inhibiting 11-beta-hydroxysteroid dehydrogenase Type I enzyme comprising administering to a mammal, a therapeutically effective amount of a compound of formula (VIII).

Another aspect of the invention includes a method of treating or prophylactically treating lipid disorders in a mammal by inhibiting 1 μl-beta-hydroxysteroid dehydrogenase Type I enzyme comprising administering to a mammal, a therapeutically effective amount of a compound of formula (IX).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (I).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (II).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (III).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (IV).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (V).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (VI).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (VII).

Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (VIII). Another aspect of the invention includes a method of treating or prophylactically treating metabolic syndrome in a mammal by inhibiting 11-beta-hydroxysteroid dehydrogenase Type I enzyme comprising administering to a mammal, a therapeutically effective amount of a compound of formula (IX).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (I).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (II).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (III).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (IV).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (V).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (VI).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (VII).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (VIII).

Another aspect of the invention includes a method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of a compound of formula (IX).

Another aspect of the present invention is directed toward a pharmaceutical composition comprising a therapeutically effective amount of a compound of formula (I) in combination with a pharmaceutically suitable carrier.

Another aspect of the present invention is directed toward a pharmaceutical composition comprising a therapeutically effective amount of a compound of formula (II) in combination with a pharmaceutically suitable carrier.

Another aspect of the present invention is directed toward a pharmaceutical composition comprising a therapeutically effective amount of a compound of formula (III) in combination with a pharmaceutically suitable carrier. Another aspect of the present invention is directed toward a pharmaceutical composition comprising a therapeutically effective amount of a compound of formula (IV) in combination with a pharmaceutically suitable carrier.

Another aspect of the present invention is directed toward a pharmaceutical composition comprising a therapeutically effective amount of a compound of formula (V) in combination with a pharmaceutically suitable carrier.

Another aspect of the present invention is directed toward a pharmaceutical composition comprising a therapeutically effective amount of a compound of formula (VI) in combination with a pharmaceutically suitable carrier.

Another aspect of the present invention is directed toward a pharmaceutical composition comprising a therapeutically effective amount of a compound of formula (VII) in combination with a pharmaceutically suitable carrier.

Another aspect of the present invention is directed toward a pharmaceutical composition comprising a therapeutically effective amount of a compound of formula (VIII) in combination with a pharmaceutically suitable carrier.

Another aspect of the present invention is directed toward a pharmaceutical composition comprising a therapeutically effective amount of a compound of formula (IX) in combination with a pharmaceutically suitable carrier.

As set forth herein, the invention includes administering a therapeutically effective amount of any of the compounds of formula I-IX and the salts and prodrugs thereof to a mamal. Preferably, the invention also includes administering a therapeutically effective amount of any of the compounds of formula I-IX to a human, and more preferably to a human in need of being treated for or prophylactically treated for any of the respective disorders set forth herein.

Definition of Terms

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkyl-NH,” as used herein, refers to an alkyl group, as defined herein, appended to the parent molecular moiety through a nitrogen atom.

The term “alkyl-NH-alkyl,” as used herein, refers to an alkyl-NH group, as defined herein, appended to the parent molecular moiety through an alkyl group, as defined herein.

The term “aryl,” as used herein, refers to a monocyclic-ring system or a polycyclic-ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention may be optionally substituted with 0, 1, 2, 3, 4 or 5 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, aryl, arylcarbonyl, aryloxy, arylsulfonyl, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, heterocycle, heterocyclecarbonyl, heterocycleoxy, heterocyclsulfonyl, hydroxy, hydroxyalkoxy, hydroxyalkyl, mercapto, mercaptoalkoxy, mercaptoalkyl, methylenedioxy, nitro, R_{f}R_{g}N—, R_{f}R_{g}Nalkyl, R_{f}R_{g}Ncarbonyl and R_{f}R_{g}Nsulfonyl, wherein R_{f} and R_{g} are members independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl, and wherein substituent aryl, the aryl of arylcarbonyl, the aryl of aryloxy, the aryl of arylsulfonyl, the substituent heterocycle, the heterocycle of heterocyclecarbonyl, the heterocycle of heterocycleoxy, the heterocycle of heterocyclesulfonyl may be optionally substituted with 0, 1, 2 or 3 substituents independently selected from the group consisting of alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, methylenedioxy, oxo, nitro, R_{f}R_{g}N—, R_{f}R_{g}Nalkyl, R_{f}R_{g}Ncarbonyl and R_{f}R_{g}Nsulfonyl.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “aryl-heterocycle,” as used herein, refers to an aryl group, as defined herein, appended to the parent molecular moiety through a heterocycle group, as defined herein.

The term “aryl-NH—,” as used herein, refers to an aryl group, as defined herein, appended to the parent molecular moiety through a nitrogen atom.

The term “aryl-NH-alkyl,” as used herein, refers to an aryl-NH— group, as defined herein, appended to the parent molecular moiety through an alkyl group, as defined herein.

The term “aryloxy,” as used herein, refers to an aryl group, as defined herein, appended to the parent molecular moiety through an oxy moiety, as defined herein. Representative examples of aryloxy include, but are not limited to phenoxy, naphthyloxy, 3-bromophenoxy, 4-chlorophenoxy, 4-methylphenoxy, and 3,5-dimethoxyphenoxy.

The term “aryloxyalkyl,” as used herein, refers to an aryloxy group, as defined herein, appended to the parent molecular moiety through an alkyl group, as defined herein.

The term “arylsulfonyl,” as used herein, refers to an aryl group, as defined herein, appended to the parent molecular moiety through a sulfonyl group, as defined herein. Representative examples of arylsulfonyl include, but are not limited to, phenylsulfonyl, 4-bromophenylsulfonyl and naphthylsulfonyl.

The term “carbonyl,” as used herein refers to a —C(O)— group.

The term “carboxy,” as used herein refers to a —C(O)—OH group.

The term “carboxyalkyl,” as used herein refers to a carboxy group as defined herein, appended to the parent molecular moiety through an alkyl group as defined herein.

The term “carboxycycloalkyl,” as used herein refers to a carboxy group as defined herein, appended to the parent molecular moiety through an cycloalkyl group as defined herein.

The term “cycloalkyl,” as used herein, refers to a saturated cyclic hydrocarbon group containing from 3 to 8 carbons. Examples of cycloalkyl include cyclopropyl, cyclobutyl, cyclopentyl, cyclohexyl, cycloheptyl, and cyclooctyl.

The cycloalkyl groups of this invention may be substituted with 1, 2, 3, 4 or 5 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, nitro, R_{f}R_{g}N—, R_{f}R_{g}Nalkyl, R_{f}R_{g}Ncarbonyl and R_{f}R_{g}Nsulfonyl, wherein R_{f} and R_{g} are members independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl.

The term “cycloalkylsulfonyl,” as used herein, refers to cycloalkyl group, as defined herein, appended to the parent molecular moiety through a sulfonyl group, as defined herein. Representative examples of cycloalkylsulfonyl include, but are not limited to, cyclohexylsulfonyl and cyclobutylsulfonyl.

The term “halo” or “halogen,” as used herein, refers to —Cl, —Br, —I or —F.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic,” as used herein, refers to a monocyclic or bicyclic ring system. Monocyclic ring systems are exemplified by any 3- or 4-membered ring containing a heteroatom independently selected from oxygen, nitrogen and sulfur; or a 5-, 6-, 7- or 8-membered ring containing one, two or three heteroatoms wherein the heteroatoms are independently members selected from nitrogen, oxygen and sulfur. The 5-membered ring has from 0-2 double bonds and the 6-, 7-, and 8-membered rings have from 0-3 double bonds. Representative examples of monocyclic ring systems include, but are not limited to, azetidinyl, azepinyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic ring systems fused to an aryl group as defined herein, a cycloalkyl group as defined herein, or another heterocyclic monocyclic ring system. Bicyclic ring systems can also be bridged and are exemplified by any of the above monocyclic ring systems joined with a cycloalkyl group as defined herein, or another non-aromatic heterocyclic monocyclic ring system. Representative examples of bicyclic ring systems include but are not limited to, for example, benzimidazolyl, benzoazepine, benzothiazolyl, benzothienyl, benzoxazolyl, benzofuranyl, benzopyranyl, benzothiopyranyl, benzodioxinyl, 1,3-benzodioxolyl, cinnolinyl, 1,5-diazocanyl, 3,9-diaza-bicyclo[4.2.1]non-9-yl, 3,7-diazabicyclo[3.3.1]nonane, octahydro-pyrrolo[3,4-c]pyrrole, indazolyl, indolyl, indolinyl, indolizinyl, naphthyridinyl, isobenzofuranyl, isobenzothienyl, isoindolyl, isoindolinyl, isoquinolinyl, phthalazinyl, pyranopyridyl, quinolinyl, quinolizinyl, quinoxalinyl, quinazolinyl, 2,3,4,5-tetrahydro-1H-benzo[c]azepine, 2,3,4,5-tetrahydro-1H-benzo[b]azepine, 2,3,4,5-tetrahydro-1H-benzo[d]azepine, tetrahydroisoquinolinyl, tetrahydroquinolinyl, and thiopyranopyridyl.

The heterocycles of this invention may be optionally substituted with 0, 1, 2 or 3 substituents independently selected from alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, aryl, arylcarbonyl, aryloxy, arylsulfonyl, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, heterocycle, heterocyclecarbonyl, heterocycleoxy, heterocyclesulfonyl, hydroxy, hydroxyalkoxy, hydroxyalkyl, mercapto, mercaptoalkoxy, mercaptoalkyl, methylenedioxy, oxo, nitro, R_{f}R_{g}N—, R_{f}R_{g}Nalkyl, R_{f}R_{g}Ncarbonyl and R_{f}R_{g}Nsulfonyl, wherein R_{f} and R_{g} are members independently selected from the group consisting of hydrogen, alkyl, alkoxyalkyl, alkylcarbonyl, alkylsulfonyl, alkoxycarbonyl, cycloalkyl, cycloalkylalkyl, cycloalkylcarbonyl and cycloalkylsulfonyl, and wherein substituent aryl, the aryl of arylcarbonyl, the aryl of aryloxy, the aryl of arylsulfonyl, the substituent heterocycle, the heterocycle of heterocyclecarbonyl, the heterocycle of heterocycleoxy, the heterocycle of heterocyclesulfonyl may be optionally substituted with 0, 1, 2 or 3 substituents independently selected from the group consisting of alkenyl, alkenylthio, alkenyloxy, alkoxy, alkoxyalkoxy, alkoxyalkoxyalkoxy, alkoxyalkoxyalkyl, alkoxyalkyl, alkoxycarbonyl, alkoxycarbonylalkoxy, alkoxycarbonylalkyl, alkoxysulfonyl, alkyl, alkylcarbonyl, alkylcarbonylalkoxy, alkylcarbonylalkyl, alkylcarbonylalkylthio, alkylcarbonyloxy, alkylcarbonylthio, alkylsulfinyl, alkylsulfinylalkyl, alkyl sulfonyl, alkylsulfonylalkyl, alkylthio, alkylthioalkyl, alkylthioalkoxy, alkynyl, alkynyloxy, alkynylthio, carboxy, carboxyalkoxy, carboxyalkyl, cyano, cyanoalkoxy, cyanoalkyl, cyanoalkylthio, ethylenedioxy, formyl, formylalkoxy, formylalkyl, haloalkenyl, haloalkenyloxy, haloalkoxy, haloalkyl, haloalkynyl, haloalkynyloxy, halogen, hydroxy, hydroxyalkoxy, hydroxyalkyl, mercapto, mercaptoalkoxy, mercaptoalkyl, methylenedioxy, oxo, nitro, R_{f}R_{g}N—, R_{f}R_{g}Nalkyl, R_{f}R_{g}Ncarbonyl and R_{f}R_{g}Nsulfonyl.

The term “heterocyclealkyl,” as used herein, refers to a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl.

The term “heterocyclealkoxy,” as used herein, refers to a heterocycle, as defined herein, appended to the parent molecular moiety through an alkoxy group, as defined herein.

The term “heterocycleoxy,” as used herein, refers to a heterocycle, as defined herein, appended to the parent molecular moiety through an oxy group, as defined herein.

The term “heterocycleoxyalkyl,” as used herein, refers to a heterocycleoxy, as defined herein, appended to the parent molecular moiety through an alkyl group, as defined herein.

The term “heterocycle-NH—,” as used herein, refers to a heterocycle, as defined herein, appended to the parent molecular moiety through a nitrogen atom.

The term “heterocycle-NH-alkyl,” as used herein, refers to a heterocycle-NH—, as defined herein, appended to the parent molecular moiety through an alkyl group, as defined herein.

The term “heterocycle-heterocycle,” as used herein, refers to a heterocycle, as defined herein, appended to the parent molecular moiety through a heterocycle group, as defined herein.

The term “heterocyclcarbonyl,” as used herein, refers to a heterocycle, as defined herein, appended to the parent molecular moiety through a carbonyl group, as defined herein. Representative examples of heterocyclecarbonyl include, but are not limited to, 1-piperidinylcarbonyl, 4-morpholinylcarbonyl, pyridin-3-ylcarbonyl and quinolin-3-ylcarbonyl.

The term “heterocyclesulfonyl,” as used herein, refers to a heterocycle, as defined herein, appended to the parent molecular moiety through a sulfonyl group, as defined herein. Representative examples of heterocyclesulfonyl include, but are not limited to, 1-piperidinylsulfonyl, 4-morpholinylsulfonyl, pyridin-3-ylsulfonyl and quinolin-3-ylsulfonyl.

The term “non-aromatic,” as used herein, refers to a monocyclic or bicyclic ring system that does not contain the appropriate number of double bonds to satisfy the rule for aromaticity. Representative examples of a “non-aromatic” heterocycles include, but are not limited to, piperidinyl, piperazinyl, homopiperazinyl, and pyrrolidinyl. Representative bicyclic ring systems are exemplified by any of the above monocyclic ring systems fused to an aryl group as defined herein, a cycloalkyl group as defined herein, or another heterocyclic monocyclic ring system.

The term “oxo,” as used herein, refers to a ═O group appended to the parent molecule through an available carbon atom.

The term “oxy,” as used herein, refers to a —O— group.

The term “sulfonyl,” as used herein, refers to a —S(O)₂— group.

Salts

The present compounds may exist as therapeutically suitable salts. The term “therapeutically suitable salt,” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use. The salts may be prepared during the final isolation and purification of the compounds or separately by reacting an amino group of the compounds with a suitable acid. For example, a compound may be dissolved in a suitable solvent, such as but not limited to methanol and water, and treated with at least one equivalent of an acid, like hydrochloric acid. The resulting salt may precipitate out and be isolated by filtration and dried under reduced pressure. Alternatively, the solvent and excess acid may be removed under reduced pressure to provide the salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, form ate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds may also be quaternized with alkyl chlorides, bromides, and iodides such as methyl, ethyl, propyl, isopropyl, butyl, lauryl, myristyl, stearyl, and the like.

Basic addition salts may be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

Prodrugs

The present compounds may also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the parent compounds of formula (1-IXc) for example, by hydrolysis in blood. The term “prodrug,” refers to compounds that contain, but are not limited to, substituents known as “therapeutically suitable esters.” The term “therapeutically suitable ester,” refers to alkoxycarbonyl groups appended to the parent molecule on an available carbon atom. More specifically, a “therapeutically suitable ester,” refers to alkoxycarbonyl groups appended to the parent molecule on one or more available aryl, cycloalkyl and/or heterocycle groups as defined herein. Compounds containing therapeutically suitable esters are an example, but are not intended to limit the scope of compounds considered to be prodrugs. Examples of prodrug ester groups include pivaloyloxymethyl, acetoxymethyl, phthalidyl, indanyl and methoxymethyl, as well as other such groups known in the art. Other examples of prodrug ester groups are found in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Optical Isomers-Diastereomers-Geometric Isomers

Asymmetric centers may exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well known in the art.

Geometric isomers may exist in the present compounds. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration. Furthermore, the invention contemplates the various isomers and mixtures thereof resulting from the disposal of substituents around an adamantane ring system. Two substituents around a single ring within an adamantane ring system are designated as being of Z or E relative configuation. For examples, see C. D. Jones, M. Kaselj, R. N. Salvatore, W. J. le Noble J. Org. Chem. 63: 2758-2760, 1998.

The compounds and processes of the present invention will be better understood in connection with the following synthetic schemes and Experimentals that illustrate a means by which the compounds of the invention may be prepared.

The compounds of this invention may be prepared by a variety of procedures and synthetic routes. Representative procedures and synthetic routes are shown in, but are not limited to, Schemes 1-5.

Abbreviations which have been used in the descriptions of the Schemes and the Examples that follow are: DCM for dichloromethane; DMAP for dimethylaminopyridine; DMF for N,N-dimethylform amide; DMSO for dimethylsulfoxide; DAST for (diethylamino)sulfur trifluoride; DIPEA or Hünig's base for diisopropylethylamine; EDCI for (3-dimethylaminopropyl)-3-ethylcarbodiimide HCl; EtOAc for ethyl acetate; EtOH for ethanol; HATU for O-(7-azabenzotriazol-1-yl)-N,N, N′,N′-tetramethyluronium hexafluoro-phosphate; HOBt for hydroxybenzotriazole hydrate; MeOH for methanol; THF for tetrahydrofuran; tosyl for para-toluene sulfonyl, mesyl for methane sulfonyl, triflate for trifluoromethane sulfonyl.

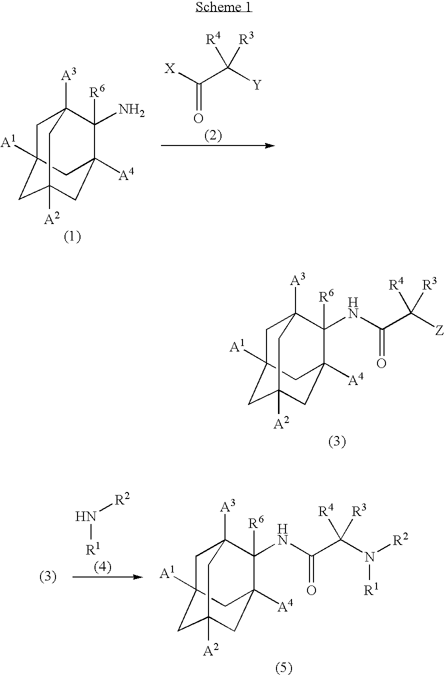

Substituted adamantanes of general formula (5), wherein A¹, A², A³, A⁴, R¹, R², R³, R⁴, and R⁶ are as defined in formula I, may be prepared as in Scheme 1. Substituted adamantamines of general formula (1), purchased or prepared using methodology known to those in the art, may be treated with acylating agents such as chloroacetyl chloride or 2-bromopropionyl bromide of general formula (2), wherein X is chloro, bromo, or fluoro, Y is a leaving group such as Cl (or a protected or masked leaving group), and R³ and R⁴ are defined as in formula I, and a base such as diisopropylethylamine to provide amides of general formula (3). Alternatively, acids of general formula (2) wherein X═OH may be coupled to substituted adamantamines of general formula (1) with reagents such as EDCI and HOBt to provide amides of general formula (3) (after conversion of Y into a leaving group Z wherein Z is chloro, bromo, iodo, —O-tosyl, —O-mesyl, or —O-triflate). Amides of general formula (3) may be treated with amines of general formula (4) wherein R¹ and R² are as defined in formula I to provide aminoamides of general formula (5). In some examples, A¹, A², A³, and/or A⁴ in amines of formula (1) may exist as a group further substituted with a protecting group such as hydroxy protected with acetyl or methoxymethyl. Examples containing a protected functional group may be required due to the synthetic schemes and the reactivity of said groups and could be later removed to provide the desired compound. Such protecting groups may be removed using methodology known to those skilled in the art or as described in T. W. Greene, P. G. M. Wuts “Protective Groups in Organic Synthesis” 3^{rd} ed. 1999, Wiley & Sons, Inc.

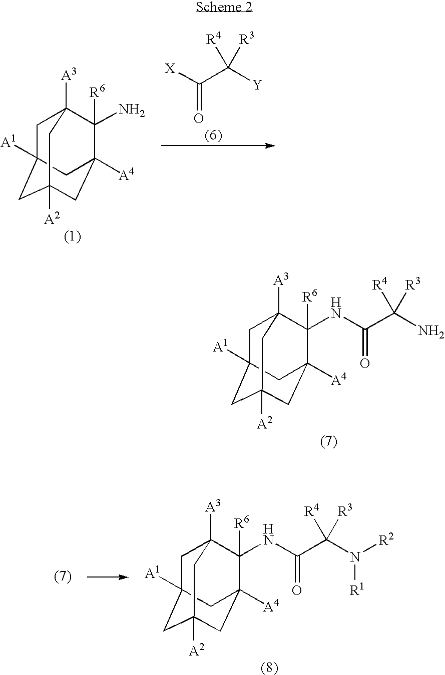

Substituted adamantanes of general formula (8), wherein A¹, A², A³, A⁴, R¹, R², R³, R⁴, and R⁶ are as defined in formula I, may be prepared as in Scheme 2. Substituted adamantamines of general formula (1) may be purchased or prepared using methodology known to those in the art. The amines of general formula (1) may be coupled with protected amino acids of general formula (6) (wherein X is OH, R³ and R⁴ are defined as in formula I, and Y is a protected or masked amino group) such as N-(tert-butoxycarbonyl)glycine with reagents such as EDCI and HOBt to provide amides of general formula (7) after deprotection. Alternatively, amines of general formula (1) may be treated with activated protected amino acids of general formula (2), wherein Y is a protected or masked amino group, and a base such as diisopropylethylamine to provide amides of general formula (7) after deprotection. Amides of general formula (7) may be treated with alkylating agents such as 1,5-dibromopentane and a base like potassium carbonate to yield amides of general formula (8).

Among other methods known to those in the art, amines of general formula (7) may be treated with aldehydes such as benzaldehyde and a reducing agent like sodium cyanoborohydride to yield amides of general formula (8). In some examples, A¹, A², A³, and/or A⁴ in amines of formula (1) may be a functional group covered with a protecting group such as hydroxy protected with acetyl or methoxymethyl. These protecting groups may be removed using methodology known to those in the art in amides of general formula (7) or (8). Alternatively a group such as chloro may be used and subsequently converted to hydroxyl by irradiating with microwaves in the presence of aqueous hydroxide.

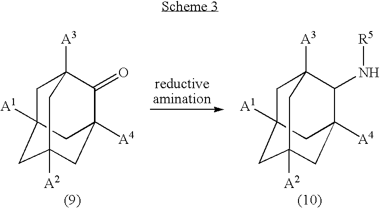

Substituted adamantane amines of general formula (10), wherein A¹, A², A³, A⁴, and R⁵ are as defined in formula I, may be prepared as in Scheme 3. Substituted adamantane ketones of general formula (9) may be purchased or prepared using methodology known to those in the art. Ketones of general formula (9) may be treated with ammonia or primary amines (R⁵NH₂) followed by reduction with sodium borohydride to provide amines of general formula (10). In some examples, A¹, A², A³, and/or A⁴ in ketones of formula (9) may be a functional group covered with a protecting group such as hydroxy protected with acetyl or methoxymethyl. These protecting groups may be removed using methodology known to those in the art in amines of general formula (10) or in compounds subsequently prepared from ketones of general formula (9) or amines of general formula (10). Alternatively a group such as chloro may be used and subsequently converted to hydroxyl by irradiating with microwaves in the presence of aqueous hydroxide.

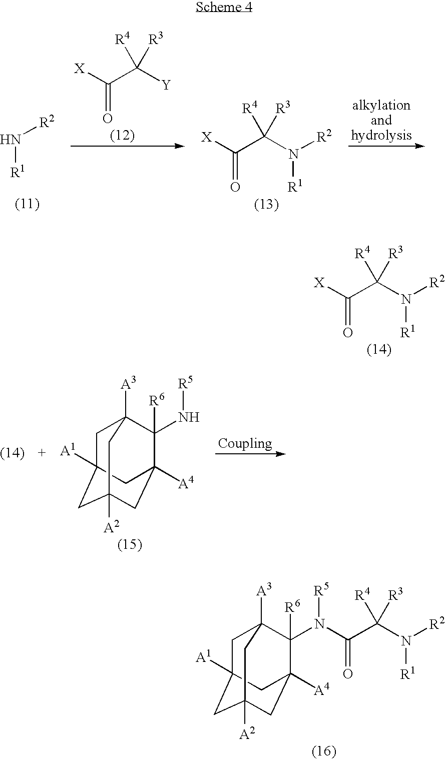

Substituted adamantanes of general formula (16), wherein A¹, A², A³, A⁴, R¹, R², R³, R⁴, R⁵, and R⁶ are as defined in formula I, may be prepared as in Scheme 4. Amines of general formula (11) may be purchased or prepared using methodology known to those in the art. The amines of general formula (11) may be reacted with reagents of general formula (12), wherein R³ and R⁴ are defined as in formula I and X is an alkoxy group, such as 2-bromopropionic acid methyl ester in the presence of a base like diisopropylethylamine to provide esters of general formula (13). Esters of general formula (13) may be alkylated using a base like lithium diisopropylamide and an alkylating agent such as methyl iodide to yield acids of general formula (14), X═OH, after hydrolysis. Substituted adamantamines of general formula (15) may be purchased or prepared using methodology known to those in the art. Coupling of acids of general formula (14) and amines of general formula (15) with reagents such as EDCI and HOBt may provide amides of general formula (16). In some examples A¹, A², A³ and/or A⁴ in amines of general formula (15) may contain a functional group such as carboxy protected with a methyl group. In amides of general formula (16), these protecting groups may be removed using methodology known to those skilled in the art.

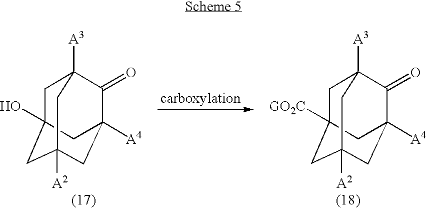

Substituted adamantanes of general formula (18), wherein A², A³, and A⁴ are as defined in formula I, may be prepared as in Scheme 5. Substituted adamantanes of general formula (17) may be purchased or prepared using methodology known to those in the art. Polycycles of general formula (17) may be treated with oleum and formic acid followed by an alcohol GOH, where G is an alkyl, cycloalkyl, aryl, or acid protecting group, to provide polycycles of general formula (18). In some examples, G in formula (9) may be a protecting group such as methyl. These protecting groups may be removed using methodology known to those in the art from polycycles of general formula (18) or in compounds subsequently prepared from (18).

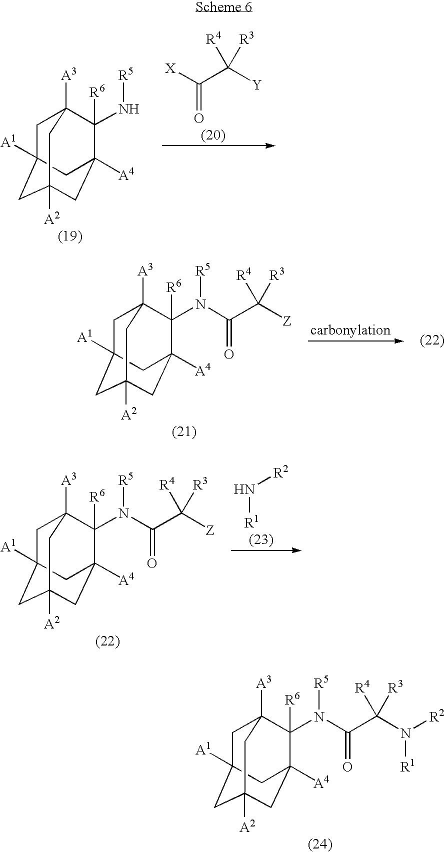

Substituted adamantanes of general formula (24), wherein A¹, A², A³, A⁴, R¹, R², R³, R⁴, R⁵, and R⁶ are as defined in formula I, may be prepared as in Scheme 6. Substituted adamantamines of general formula (19), wherein A¹, A², A³, and A⁴ are defined as in formula one I with the proviso that at least one is a hydroxyl group or a protected or masked hydroxyl group, may be purchased or prepared using methodology known to those in the art. Substituted adamantamines of general formula (19) may be treated with acylating agents such as chloroacetyl chloride or 2-bromopropionyl bromide of general formula (20), wherein X is chloro, bromo, or fluoro, Y is a leaving group such as Cl (or a protected or masked leaving group), and R³ and R⁴ are defined as in formula I, and a base such as diisopropylethylamine to provide amides of general formula (21). Alternatively, acids of general formula (20) wherein X═OH may be coupled to substituted adamantamines of general formula (19) with reagents such as EDCI and HOBt to provide amides of general formula (21) (after conversion of Y into a leaving group Z wherein Z is chloro, bromo, iodo, —O-tosyl, —O-mesyl, or —O-triflate). Hydroxyadamantanes, or protected or masked hydroxyl adamantanes which can be converted to the corresponding hydroxyadamantane, (21) may be carbonylated with reagents like oleum and formic acid to yield the corresponding adamantyl acid or ester (22), wherein A¹, A², A³, and A⁴ are defined as in formula one I with the proviso that at least one is a carboxy group or a protected carboxy group (CO₂R¹⁷ wherein R¹⁷ is defined as in formula I). Amides of general formula (22) may be treated with amines of general formula (23) wherein R¹ and R² are as defined in formula I to provide aminoamides of general formula (24). In some examples, A¹, A², A³, and/or A⁴ in amines of formula (24) may exist as a group further substituted with a protecting group such as carboxy protected as an alkyl ester. Examples containing a protected functional group may be required due to the synthetic schemes and the reactivity of said groups and could be later removed to provide the desired compound. Such protecting groups may be removed using methodology known to those skilled in the art or as described in T. W. Greene, P. G. M. Wuts “Protective Groups in Organic Synthesis” 3^{rd} ed. 1999, Wiley & Sons, Inc.

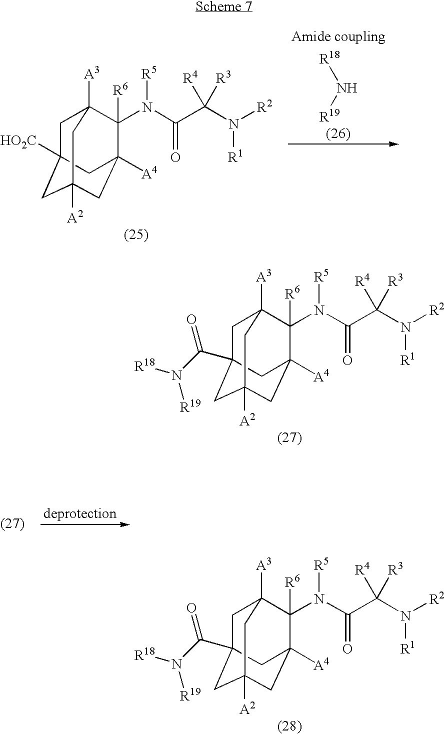

Substituted adamantanes of general formula (28), wherein A², A³, A⁴, R¹, R², R³, R⁴, R⁵, R⁶, R¹⁸, and R¹⁹ are as defined in formula I, may be prepared as in Scheme 7. Adamantyl acids of general formula (25) may be prepared as described herein or using methodology known to those in the art. The acids of general formula (25) may be coupled with amines of general formula (26) (wherein R¹⁸ and R¹⁹ are defined as in formula I) with reagents such as O-(Benzotrialzol-1-yl)-1,1,3,3-tetramethyluronium tetrafluoroborate (TBTU) to provide amides of general formula (27). In some examples, A², A³, A⁴, R¹, R², R³, R⁴, R⁵, R⁶, R¹⁸, and R¹⁹ in amines of formula (27) may contain a functional group covered with a protecting group such as carboxy protected as an ester. These protecting groups may be removed using methodology known to those in the art to provide amides of general formula (28).

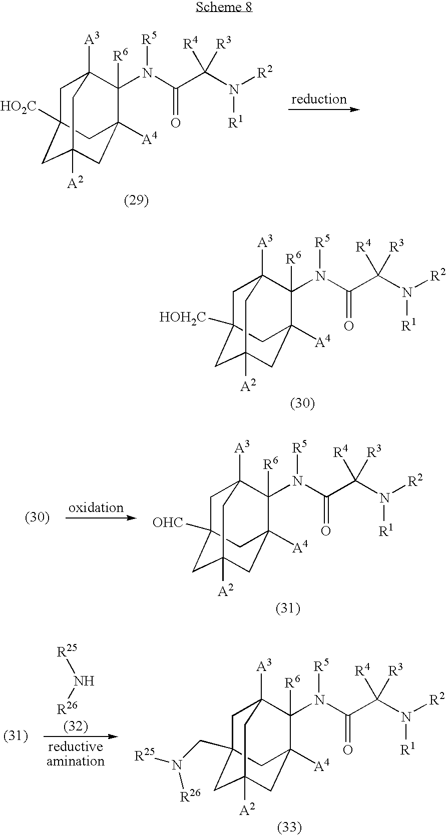

Substituted adamantanes of general formula (33), wherein A 2, A¹, A⁴, R¹, R², R³, R⁴, R⁵, R⁶, R²⁵, and R²⁶ are as defined in formula I, may be prepared as in Scheme 8. Acids of general formula (29) may be prepared as detailed herein or by using methodology known to those in the art. Acids (29) may be reduced using a reagent like borane to alcohols of general formula (30). Alcohols of general formula (30) may be oxidized with reagents such as tetrapropylammonium perruthenate to aldehydes of general formula (31). Aldehydes of general formula (31) may be reductively aminated with an amine of general formula (32), wherein R²⁵ and R²⁶ are as defined in formula I, and a reducing agent such as sodium cyanoborohydride to provide amines of general formula (33). In some examples, A², A³, A⁴, R¹, R², R³, R⁴, R⁵, R⁶, R²⁵, and R²⁶ in amines of formula (33) may be and/or contain a functional group covered with a protecting group such as such as carboxy protected as an ester. These protecting groups may be removed using methodology known to those in the art.

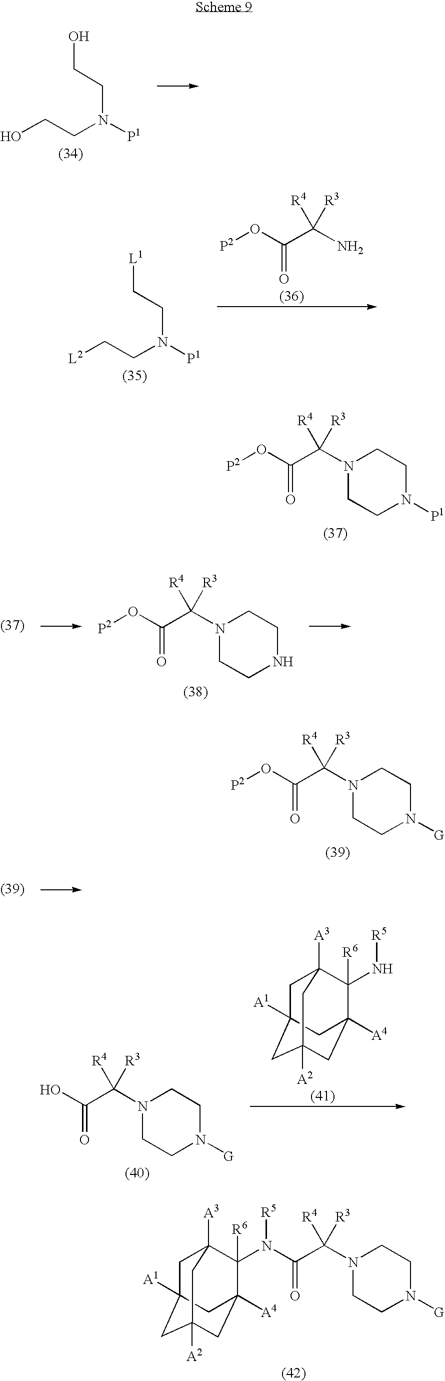

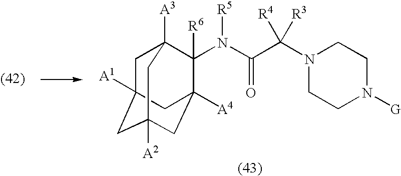

Substituted adamantanes of general formula (42), wherein A¹, A², A³, A⁴, R³, R⁴, R⁵, and R⁶ are as defined in formula I and G is as defined in formula V, may be prepared as in Scheme 9. Diethanolamines of general formula (34) wherein P¹ is an alkylsulfonyl or arylsulfonyl group may be purchased or prepared using methodology known to those in the art. Diethanolamines (34) wherein P¹ is an alkylsulfonyl or arylsulfonyl group can be prepared by reacting diethanolamine with a sulfonyl chloride like 2-nitrobenzenesulfonylchloride in the presence of a base like triethylamine in a solvent like methylene chloride. The diols of general formula (34) may be converted to sulfonamides of general formula (35) (wherein L¹ and L² are Cl, Br, I, OMs, or OTf) with reagents such as triflic anhydride. Sulfonamides of general formula (35) may be treated with aminoesters (36), wherein R³ and R⁴ are as defined in formula I and p² is an alkyl group, and a base like sodium carbonate to yield piperazines of general formula (37). Piperazine sulfonamides (37) can be deprotected to provide piperazines (38). Amines (38) can be arylated, or heteroarylated, with a reagent like 2-bromo-5-trifluoromethyl-pyridine to give piperazines of general formula (39). Esters (39) may be converted to acids of general formula (40). Acids (40) can be coupled to adamantly amines of general formula (41), wherein A¹, A², A³, A⁴, and R⁶ are as defined in formula I, to give amides of general formula (42). In some examples, A¹, A², A³, A⁴, R³, R⁴, R⁵, and/or R⁶ in amines of formula (42) may contain a functional group covered with a protecting group such as such as carboxy protected as an ester. These protecting groups may be removed using methodology known to those in the art to give amides of general formula (43).

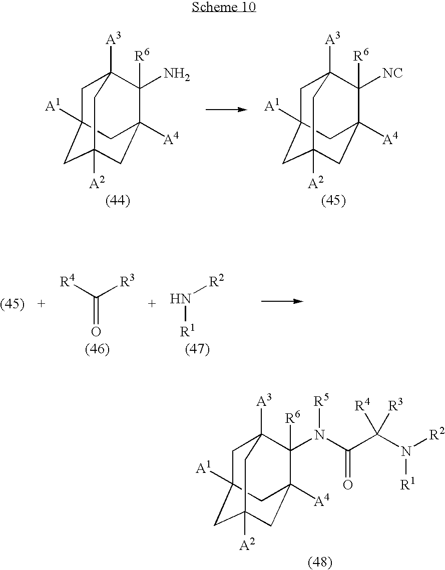

Substituted adamantanes of general formula (48), wherein A¹, A², A³, A⁴, R¹, R², R³, R⁴, R⁵, and R⁶ are as defined in formula I, may be prepared as in Scheme 10. Substituted adamantamines of general formula (44), wherein A¹, A², A³, A⁴, and R⁶ are as defined in formula I, may be purchased or prepared using methodology known to those in the art. The amines of general formula (44) may be converted to isonitriles of general formula (45) with reagents such as methyl formate followed by treatment with phosphorous oxychloride in the presence of a base like triethylamine. Isonitriles of general formula (45) may be treated with aldehydes or ketones of general formula (46), amines of general formula (47), and an acid such as acetic acid to provide amides of general formula (48). In some examples, A¹, A², A³, A⁴, R¹, R², R³, R⁴, R⁵, and/or R⁶ in compounds of formula (48) may contain a functional group covered with a protecting group such as carboxy protected as an ester. These protecting groups may be removed using methodology known to those in the art in amides of general formula (48).

The compounds and processes of the present invention will be better understood by reference to the following Examples, which are intended as an illustration of and not a limitation upon the scope of the invention. Further, all citations herein are incorporated by reference.

Compounds of the invention were named by ACD/ChemSketch version 5.01 (developed by Advanced Chemistry Development, Inc., Toronto, ON, Canada) or were given names consistent with ACD nomenclature. Adamantane ring system isomers were named according to common conventions. Two substituents around a single ring within an adamantane ring system are designated as being of Z or E relative configuation (for examples see C. D. Jones, M. Kaselj, R. N. Salvatore, W. J. le Noble J. Org. Chem. 63: 2758-2760, 1998).

### EXAMPLE 1

### N-[(Z)-5-hydroxy-2-adamantyl]-2-{4-[5-(trifluoromethyl)pyridin-2-yl]piperazin-1-yl}acetamide

### EXAMPLE 1A

### Acetic acid 2-oxo-adamantan-5-yl ester

A solution of 5-hydroxy-2-adamantanone (2.6 g, 15.66 mmoles) in dichloromethane (DCM) (50 mL) was treated with dimethylaminopyridine (DMAP) (2.1 g, 17 mmoles) and acetic anhydride (2.3 mL, 23 mmoles) and stirred overnight at 50° C. The solvent was removed under reduced pressure and the residue was partitioned between water and ethyl acetate. The aqueous layer was extracted twice with ethyl acetate. Combined organic extracts were washed with water, dried (MgSO₄) and filtered. The filtrate was concentrated under reduced pressure to provide the title compound as an off-white solid (3.124 g, 95.8%).

### EXAMPLE 1B

### E- and Z-acetic acid 2-amino-adamantan-5-yl ester

A solution of acetic acid 2-oxo-adamantan-5-yl ester (3.124 g, 15 mmoles), from Example 1A, and 4 Å molecular seives (1 g) in methanolic ammonia (7N, 50 mL) was stirred overnight at room temperature. The mixture was cooled in an ice bath, treated portionwise with sodium borohydride (2.27 g, 60 mmoles) and stirred at room temperature for 2 hours. The suspension was filtered and concentrated under reduced pressure. The residue was taken into DCM (50 mL), acidified with 1N HCl to pH=3 and the layers separated. The aqueous layer was basified with 2N NaOH to pH=12 and extracted three times with 4:1 tetrahydrofuran:dichloromethane (THF:DCM). The combined organic extracts were dried (MgSO₄) and filtered. The filtrate was concentrated under reduced pressure to provide the title compound as a white solid (1.82 g, 58%).

### EXAMPLE 1C

### E- and Z-acetic acid 2-(2-chloroacetylamino)-adamantan-5-yl ester

A solution of E- and Z-acetic acid 2-amino-adamantan-5-yl ester (1.82 g, 8.69 mmoles), from Example 1B, in DCM (30 mL) and diisopropylethylamine (DIPEA) (1.74 mL, 10 mmoles) was cooled in an ice bath and treated with chloroacetyl chloride (0.76 mL, 9.57 mmoles). The solution was stirred for 2 hours at room temperature and concentrated under reduced pressure. The residue was partitioned between water and ethyl acetate. The organic layer was washed with saturated aqueous sodium bicarbonate, water, dried (MgSO₄) and filtered. The filtrate was concentrated under reduced pressure to provide the title compound as dark beige solid (2.1 g, 84.5%).

### EXAMPLE 1D

A solution of E- and Z-acetic acid 2-(2-chloroacetylamino)-adamantan-5-yl ester (2.1 g, 7.3 mmoles), from Example 1C, in MeOH (30 mL) and DIPEA (1.53 mL, 8.8 mmoles) was treated with 1-(5-trifluoromethyl-pyridin-2-yl)-piperazine (2.04 g, 8.8 mmoles) and stirred for 6 hours at 70° C. An aqueous solution of potassium carbonate (K₂CO₃) (15 mL) was added to the reaction and stirred overnight at 70° C. MeOH was removed under reduced pressure and the residue was partitioned with DCM. The aqueous layer was extracted with DCM and the combined organic extracts washed twice with water, dried (MgSO₄) and filtered. The filtrate was concentrated under reduced pressure to provide an off-white solid, which was purified by column chromatography (silica gel, 30-90% acetone in hexane) to provide the title compound as a white solid (0.5 g, 23%). ¹H NMR (300 MHz, CDCl₃) δ 8.41 (s, 1H), 7.65 (dd, J=2.7, 9.1 Hz, 1H,), 7.6 (s, 1H), 6.65 (d, J=9.1 Hz, 1H), 3.98 (d, J=8.5 Hz, 1H), 3.69 (s, 4H), 3.09 (s, 2H), 2.67 (s, 4H), 2.19-2.15 (m, 3H), 1.79-1.38 (m, 10H); MS (APCI+) m/z 439 (M+H)⁺.

### EXAMPLE 2

### N-[(E)-5-hydroxy-2-adamantyl]-2-{4-[5-(trifluoromethyl)pyridin-2-yl]piperazin-1-yl}acetamide

Purification of the concentrated filtrate from Example 1D by column chromatography (silica gel, 30-90% acetone in hexane) provided the title compound as a white solid (1.5 g, 47%). ¹H NMR (300 MHz, CDCl₃) δ 8.41 (s, 1H), 7.67 (dd, J=2.1, 9.1 Hz, 1H), 7.6 (s, 1H), 6.67 (d, J=9.1 Hz, 1H), 4.07 (d, J=8.1 Hz, 1H), 3.69 (s, 4H), 3.1 (s, 2H), 2.68 (s, 4H), 2.12-2.17 (m, 3H), 1.91 (m, 2H), 1.79-1.75 (m, 4H), 1.67 (m, 2H), 1.57 (s, 1H), 1.36 (s, 1H); MS (APCI+) m/z 439 (M+H)⁺.

### EXAMPLE 3

### N-[(E)-5-hydroxy-2-adamantyl]-2-{4-[5-(trifluoromethyl)pyridin-2-yl]piperazin-1-yl}propanamide

### EXAMPLE 3A

### E- and Z-acetic acid 2-(2-bromo-propionylamino)-adamantan-5-yl ester

A solution of E- and Z-acetic acid 2-amino-adamantan-5-yl ester (0.54 g, 2.58 mmoles), from Example 1B, in DCM (10 mL) and DIPEA (0.54 mL, 3.09 mmoles) was cooled in an ice bath and treated with 2-bromopropionyl chloride (0.26 mL, 2.6 mmoles). The solution was stirred for 2 hours at room temperature and DCM was removed under reduced pressure. The residue was partitioned between water and ethyl acetate. The organic layer was washed with saturated aqueous sodium bicarbonate, water, dried (MgSO₄) and filtered. The filtrate was concentrated under reduced pressure to provide the title compound as a dark beige solid (746 mg, 84%).

### EXAMPLE 3B

A solution of E- and Z-acetic acid 2-(2-bromo-propionylamino)-adamantan-5-yl ester (0.746 g, 2.17 mmoles), from Example 3A, in MeOH (10 mL) and DIPEA (0.416 mL, 2.39 mmoles) was treated with 1-(5-trifluoromethyl-pyridin-2-yl)-piperazine (0.552 g, 2.39 mmoles) and stirred for 6 hours at 70° C. Saturated aqueous K₂CO₃ (5 mL) was added to the reaction mixture and the mixture stirred overnight at 70° C. The mixture was concentrated under reduced pressure and the residue partitioned by the addition of DCM. The aqueous layer was extracted with additional DCM (3×). The combined organic extracts were washed twice with water, dried (MgSO₄) and filtered. The filtrate was concentrated under reduced pressure to provide an off-white solid, which was purified by column chromatography (silica gel, 30-90% acetone in hexane) to provide the title compound as a white solid (500 mg, 53%). ¹H NMR (300 MHz, CDCl₃) δ 8.41 (s, 1H), 7.65 (m, 2H), 6.67 (d, J=8.8 Hz, 1H), 4.03 (d, J=8.5 Hz, 1H), 3.69 (m, 4H), 3.15 (q, J=7.1 Hz, 1H), 2.63 (m, 4H), 2.15 (m, 3H), 1.9 (m, 2H), 1.77 (m, 4H), 1.66 (m, 2H), 1.52 (s, 1H), 1.36 (s, 1H), 1.28 (d, J=7.1 Hz, 3H); MS(APCI+) m/z 453 (M+H)⁺.

### EXAMPLE 4

### 2-[(cis)-2,6-dimethylmorpholin-4-yl]-N-[(E)-5-hydroxy-2-adamantyl]propanamide

### EXAMPLE 4A

### E- and Z-5-chloro-2-adamantamine

A solution of 5-chloro-2-adamantanone (4.8 g, 26 mmoles) and 4 Å molecular sieves (2 g) in methanolic ammonia (7N, 50 mL) was stirred overnight at room temperature, cooled in an ice bath, treated with the portionwise addition of sodium borohydride (3.93 g, 104 mmoles) and stirred at room temperature for 2 hours. The suspension was filtered and concentrated under reduced pressure. The residue was taken into DCM (50 mL) and acidified with 1N HCl to pH=3. The layers were separated and the aqueous layer basified with 2N NaOH to pH=12 and extracted three times with 4:1 THF:DCM. The combined organic extracts were dried (MgSO₄), filtered and concentrated under reduced pressure to provide the title compound as a white solid (4.68 g, 97%).

### EXAMPLE 4B

### E- and Z-2-bromo-N-(5-chloro-adamantan-2-yl)-propionamide

A solution of E- and Z-5-chloro-2-adamantamine (1 g, 5.38 mmoles), from Example 4A, in DCM (30 mL) and DIPEA (2.08 mL, 11.96 mmoles) was cooled in an ice bath and treated with 2-bromopropionyl chloride (0.65 mL, 6.46 mmoles) and the mixture stirred for 2 hours at room temperature. The mixture was concentrated under reduced pressure, partitioned between water and ethyl acetate. The organic layer was washed with aqueous saturated sodium bicarbonate (2×), water (2×), dried (MgSO₄) and filtered. The filtrate was concentrated under reduced pressure to provide the title compound as a tan solid (1.47 g, 85%).

### EXAMPLE 4C

A solution of E- and Z-2-bromo-N-(5-chloro-adamantan-2-yl)-propionamide (55 mg, 0.17 mmoles) from Example 4B in MeOH (1 mL) and DIPEA (0.1 mL) was treated with cis-2,6-dimethylmorpholine (23 mg, 0.2 mmoles) and the mixture stirred overnight at 70° C. The mixture was concentrated under reduced pressure. The residue dissolved in dioxane (0.1 mL) and 5N potassium hydroxide (0.4 mL) and irradiated by microwaves for 1 hour at 190° C. The mixture was filtered through a Celite cartridge and washed with 1:1 DMSO:MeOH (1.5 mL). The title compound was isolated by reverse phase HPLC (20-100% acetonitrile in 0.1% TFA in water) on a YMC ODS Guardpak column as a clear oil (30 mg, 48%). ¹H NMR (300 MHz, CDCl₃) δ 7.65 (d, J=8.3 Hz, 1H); 4.0 (d, J=8.6 Hz, 1H), 3.67 (m, 2H), 3.03 (q, J=7.0 Hz, 1H), 2.62 (t, J=11.2 Hz, 2H), 2.11 (m, 3H), 1.97-1.8 (m, 3H), 1.77-1.65 (m, 4H), 1.65-1.52 (m, 4H), 1.23 (d; J=7.1 Hz, 3H), 1.17 (dd, J=5.8, 6.1 Hz, 6H); MS (APCI+) m/z 337 (M+H)⁺.

### EXAMPLE 5

### N-[(Z)-5-hydroxy-2-adamantyl]-2-(4-hydroxypiperidin-1-yl)propanamide

The title compound was prepared according to the method of Example 4C substituting 4-hydroxypiperidine for cis-2,6-dimethylmorpholine (12 mg, 21%). ¹H NMR (300 MHz, CDCl₃) δ 7.75 (s, 1H), 3.9 (d, J=9.2 Hz, 1H), 3.74 (s, 1H), 3.12 (m, 1H), 2.77 (m, 2H), 2.43 (m, 1H), 2.25 (m, 2H), 2.15-1.93 (m, 10H), 1.75-1.6 (m, 8H), 1.23 (d, J=6.8 Hz, 3H); MS(APCI+) m/z 323 (M+H)⁺.

### EXAMPLE 6

### N-[(E)-5-hydroxy-2-adamantyl]-2-(4-hydroxypiperidin-1-yl)propanamide

The title compound was prepared according to the method of Example 4C substituting 4-hydroxypiperidine for cis-2,6-dimethylmorpholine (24 mg, 42%). ¹H NMR (300 MHz, CDCl₃) δ 7.76 (d, J=2.4 Hz, 1H), 4.0 (d, J=8.1 Hz, 1H), 3.74 (m, 1H), 3.13 (q, J=7.2 Hz, 1H), 2.78 (m, 2H), 2.44 (t, 12.2, 1H), 2.28 (t, J=9.6 Hz, 1H), 2.16-2.05 (m, 5H), 1.96-1.88 (m, 4H), 1.77-1.52 (m, 9H), 1.23 (d, J=7.2 Hz, 3H); MS(APCI+) m/z 323 (M+H)⁺.

### EXAMPLE 7

### 2-azepan-1-yl-N-[(E)-5-hydroxy-2-adamantyl]propanamide

The title compound was prepared according to the method of Example 4C substituting hexamethyleneimine for cis-2,6-dimethylmorpholine (35 mg, 60%). ¹H NMR (300 MHz, CDCl₃) δ 7.84 (s, 1H), 3.99 (d, J=8.1 Hz, 1H), 3.35 (d, J=5.9 Hz, 1H), 2.71-2.65 (bd, 4H), 2.16-2.10 (m, 3H), 1.89 (d, J=11.9 Hz, 2H), 1.77-1.65 (m, 14H), 1.52 (d, J=12.8 Hz, 2H), 1.24 (d, J=6.9 Hz, 3H); MS(APCI+) m/z 321 (M+H)⁺.

### EXAMPLE 8

### (E)-4-[({4-[5-(trifluoromethyl)pyridin-2-yl]piperazin-1-yl}acetyl)amino]-1-adamantyl carbamate

A solution of N-[(E)-5-hydroxy-2-adamantyl]-2-{4-[5-(trifluoromethyl)pyridin-2-yl]piperazin-1-yl}acetamide (44 mg, 0.1 mmoles) from Example 2 in DCM (1 mL) was treated with trichloroacetylisocyanate (13 μL, 0.11 mmoles) and stirred for 2 hours at room temperature. The solvent was removed under reduced pressure, the residue was dissolved in MeOH (1 mL) followed by the addition of saturated potassium carbonate (3 mL) and the mixture stirred overnight at 50° C. The mixture was concentrated under reduced pressure, partitioned with DCM and the aqueous layer extracted with additional DCM. The combined organic extracts were washed twice with water, dried (MgSO₄) and filtered. The filtrate was concentrated under reduced pressure to provide the title compound as a white solid (40 mg, 83%). ¹H NMR (300 MHz, CDCl₃) δ 8.42 (s, 1H), 7.64 (m, 2H), 6.67 (d, J=9.2 Hz, 1H), 4.4 (s, 2H), 4.12 (d, J=5.8 Hz, 1H), 3.68 (s, 4H), 3.09 (s, 2H), 2.68 (s, 4H), 2.19-2.17 (m, 9H), 1.64-1.63 (m, 4H); MS (APCI+) m/z 482 (M+H)⁺.

### EXAMPLE 9

### (E)-4-[(2-{4-[5-(trifluoromethyl)pyridin-2-yl]piperazin-1-yl}acetyl)amino]-1-adamantyl acetate

A solution of N-[(E)-5-hydroxy-2-adamantyl]-2-{4-[5-(trifluoromethyl)pyridin-2-yl]piperazin-1-yl}acetamide (44 mg, 0.1 mmoles) from Example 2 in DCM (0.5 mL) and pyridine (0.5 mL) was treated with acetyl chloride (11 μL, 0.15 mmoles), catalytic amount of DMAP and stirred overnight at 50° C. Solvents were removed under reduced pressure and the residue was purified (silica gel, 10-30% acetone in hexane) to provide the title compound as a white solid (35 mg, 73%). ¹H NMR (300 MHz, CDCl₃) δ 8.41 (s, 1H), 7.64 (m, 2H), 6.65 (d, J=9.2 Hz, 1H), 4.12 (d, J=8.1 Hz, 1H), 3.68 (s, 4H), 3.09 (s, 2H), 2.68 (s, 4H), 2.21-2.14 (m, 7H), 1.98 (s, 3H), 1.64 (s, 2H), 1.26-1.22 (m, 4H); MS (APCI+) m/z 481 (M+H)⁺.

### EXAMPLE 10

### N-[(E)-5-(acetylamino)-2-adamantyl]-2-{4-[5-(trifluoromethyl)pyridin-2-yl]piperazin-1-yl}acetamide

A solution of N-[(E)-5-hydroxy-2-adamantyl]-2-{4-[5-(trifluoromethyl)pyridin-2-yl]piperazin-1-yl}acetamide (44 mg, 0.1 mmoles) from Example 2 in TFA (0.5 mL) and acetonitrile (0.1 mL) was stirred overnight at 100° C. The mixture was adjusted to pH 10 with 2N NaOH and extracted with DCM. The organic layer was washed with water (2×), dried (MgSO₄) and filtered. The filtrate was concentrated under reduced pressure and purified (silica gel, 10-35% acetone in hexane) to provide the title compound as a white solid (38 mg, 79%). ¹H NMR (300 MHz, CDCl₃) δ 8.41 (s, 1H), 7.64 (m, 2H), 6.67 (d, J=9 Hz, 1H), 5.16 (s, 1H), 4.10 (d, J=8.4 Hz, 1H), 3.69 (s, 4H), 3.09 (s, 2H), 2.68 (s, 4H), 2.18-2.16 (d, 2H), 2.09 (d, 4H), 2.01 (d, 2H), 1.92 (s, 3H), 1.69-1.63 (m, 5H); MS (APCI+) m/z 480 (M+H)⁺.

### EXAMPLE 11

### N-[(E)-5-fluoro-2-adamantyl]-2-{4-[5-(trifluoromethyl)pyridin-2-yl]piperazin-1-yl}acetamide

A solution of N-[(E)-5-hydroxy-2-adamantyl]-2-{4-[5-(trifluoromethyl)pyridin-2-yl]piperazin-1-yl}acetamide (66 mg, 0.15 mmoles) from Example 2 in DCM (0.5 mL) was cooled to −78° C., treated with (diethylamino)sulfur trifluoride (DAST) (0.020 mL, 0.16 mmoles) and slowly warmed to room temperature over 6 hours. The mixture was quenched with aqueous saturated sodium bicarbonate (0.1 mL), filtered through a Celite cartridge and purified (silica gel, 10-15% acetone in hexane) to provide the title compound as a white solid (42 mg, 63%). ¹H NMR (300 MHz, CDCl₃) δ 8.42 (s, 1H), 7.63 (m, 2H), 6.68 (d, J=9.2 Hz, 1H), 4.09 (d, J=8.5 Hz, 1H), 3.69 (s, 4H), 3.09 (s, 2H), 2.69 (s, 4H), 2.27-2.22 (m, 3H), 2.06 (m, 2H), 1.94 (m, 4H), 1.58-1.54 (m, 4H); (APCI+) m/z 441 (M+H)⁺.

### EXAMPLE 12

### N-[(Z)-5-fluoro-2-adamantyl]-2-{4-[5-(trifluoromethyl)pyridin-2-yl]piperazin-1-yl}acetamide

A solution of N-[(Z)-5-hydroxy-2-adamantyl]-2-{4-[5-(trifluoromethyl)pyridin-2-yl]piperazin-1-yl}acetamide (66 mg, 0.15 mmoles) from Example 1D in DCM (0.5 mL) was cooled to −78° C., treated with DAST (0.020 mL, 0.16 mmoles) and slowly warmed to room temperature for 6 hours. The mixture was quenched by the addition of aqueous saturated sodium bicarbonate (0.1 mL), filtered through a Celite cartridge and purified (silica gel, 10-15% acetone in hexane) to provide the title compound as a white solid (40 mg, 62%). ¹H NMR (300 MHz, CDCl₃) δ 8.42 (s, 1H), 7.67 (m, 2H), 6.67 (d, J=9.1 Hz, 1H), 3.97 (s, 1H), 3.7 (s, 4H), 3.1 (s, 2H), 2.68 (s, 4H), 2.29-2.24 (m, 3H), 1.91-1.7 (m, 10H); MS (APCI+) m/z 441 (M+H)⁺.

### EXAMPLE 13

### N-[(E)-5-hydroxy-2-adamantyl]-2-[4-(5-methylpyridin-2-yl)piperazin-1-yl]propanamide

### EXAMPLE 13A E- and Z-5-hydroxy-2-adamantamine

A solution of 5-hydroxy-2-adamantanone (10 g, 60.161 mmoles) and 4 Å molecular sieves (5 g) in methanolic ammonia (7N, 100 mL) was stirred overnight at room temperature. The mixture was cooled in an ice bath, treated by the portionwise addition of sodium borohydride (9.1 g, 240.64 mmoles) and stirred at room temperature for 2 hours. The mixture was filtered and MeOH was removed under reduced pressure. The mixture was taken into DCM (100 mL), acidified with 1N HCl to pH=3 and the layers separated. The aqueous layer was treated with 2N NaOH solution to pH=12 and extracted three times with 4:1 THF:DCM. The combined organic extracts were dried (MgSO₄) and filtered. The filtrate was concentrated under reduced pressure to provide the title compound as a white solid (9.84 g, 97.9%).

### EXAMPLE 13B

### E- and Z-2-bromo-N-(5-hydroxy-adamantan-2-yl)-propionamide

A solution of E- and Z-5-hydroxy-2-adamantamine (1 g, 5.98 mmoles) from Example 13A in DCM (30 mL) and DIPEA (2.08 mL, 11.96 mmoles) was cooled in an ice bath and treated with 2-bromopropionyl chloride (0.66 mL, 6.58 mmoles). The mixture was stirred for 2 hours at room temperature and DCM was removed under reduced pressure. The residue was partitioned between water and ethyl acetate. The organic layer was washed with saturated sodium bicarbonate, water, dried (MgSO₄) and filtered. The filtrate was concentrated under reduced pressure to provide the title compound as a dark beige solid (1.53 g, 84.6%). The isomers were separated by column chromatography (silica gel, 5-35% acetone in hexane) to furnish 1 g of E-2-bromo-N-(5-hydroxy-adamantan-2-yl)propionamide and 0.5 g of Z-2-bromo-N-(5-hydroxy-adamantan-2-yl)propionamide.

### EXAMPLE 13C

### 1-(5-Methyl-pyridin-2-yl)-piperazine

A solution of piperazine (215 mg, 2.5 mmoles), 2-bromo-5-methyl-pyridine (172 mg, 1 mmoles) in dioxane (1 mL) and potassium carbonate (276 mg, 2 mmoles) was irradiated by microwaves for 60 minutes at 180° C. The dioxane was removed under reduced pressure and the residue partitioned between aqueous potassium carbonate and ethyl acetate. The aqueous layer was extracted with ethyl acetate and the combined organic extracts washed twice with water, dried (MgSO₄) and filtered. The filtrate was concentrated under reduced pressure and the residue was purified (silica gel, 0-10% methanol in dichloromethane) to provide the title compound as a white solid (140 mg, 79%).

### EXAMPLE 13D

A solution of E-2-bromo-N-(5-hydroxy-adamantan-2-yl)-propionamide (36 mg, 0.12 mmoles) from Example 13B and 1-(5-methyl-pyridin-2-yl)-piperazine (21 mg, 0.12 mmoles) from Example 13C in MeOH (0.5 mL) and DIPEA (0.1 mL) was stirred overnight at 70° C. The MeOH was removed under reduced pressure and the residue purified (silica gel, 10-40% acetone in hexane) to provide the title compound as a white solid (40 mg, 83%). ¹H NMR (300 MHz, CDCl₃) δ 8.06 (d, J=5.3, 1H), 7.71 (s, 1H), 6.51 (s, 2H), 4.02 (d, J=8.2 Hz, 1H), 3.56 (s, 4H), 3.12 (m, 1H), 2.68 (bd, 4H), 2.28 (s, 3H), 2.17-2.10 (m, 3H), 1.91-1.88 (d, J=11.5 Hz, 2H), 1.76 (s, 4H), 1.66 (d, J=12.5 Hz, 2H), 1.51 (m, 2H), 1.27 (m, 3H); MS (APCI+) m/z 399 (M+H)⁺.

### EXAMPLE 14

### N-[(E)-5-hydroxy-2-adamantyl]-2-methyl-2-{4-[5-(trifluoromethyl)pyridin-2-yl]piperazin-1-yl}propanamide

### EXAMPLE 14A

### 2-[4-(5-Trifluoromethyl-pyridin-2-yl)-piperazin-1-yl]-propionic acid methyl ester

A solution of 1-(5-trifluoromethyl-pyridin-2-yl)-piperazine (0.9 g, 3.9 mmoles) in MeOH (13 mL) and DIPEA (1.5 mL) was treated with 2-bromo-propionic acid methyl ester (0.48 mL, 4.3 mmoles) and stirred overnight at 70° C. The MeOH was removed under reduced pressure and the residue was purified (silica gel, 10-40% acetone in hexane) to provide the title compound as a yellowish solid (1.23 g, 99%).

### EXAMPLE 14B 2-Methyl-2-[4-(5-trifluoromethyl-pyridin-2-yl)-piperazin-1-yl]-propionic acid methyl ester

A solution of 2-[4-(5-trifluoromethyl-pyridin-2-yl)-piperazin-1-yl]-propionic acid methyl ester (1.23 g, 3.9 mmoles) from Example 14A in dry THF (3 mL) was added dropwise to a −65° C. solution of 1.8 N lithium diisopropylamine (LDA) in dry THF (2.4 mL) and stirred at this temperature for 1 hour. Methyl iodide (0.49 mL, 7.88 mmoles) was added and the mixture was allowed to slowly warm to room temperature and stir for 2 hours at room temperature. The mixture was quenched with ice/water and partitioned with ethyl acetate. The aqueous layer was extracted with ethyl acetate (3×) and the combined organic extracts washed with water, dried (MgSO₄), filtered and the filtrate concentrated under reduced pressure. The residue was purified (silica gel, 10-30% acetone in hexane) to provide the title compound as a yellowish solid (1.05 g, 81.7%).

### EXAMPLE 14C 2-Methyl-2-[4-(5-trifluoromethyl-pyridin-2-yl)-piperazin-1-yl]-propionic acid

A solution of 2-methyl-2-[4-(5-trifluoromethyl-pyridin-2-yl)-piperazin-1-yl]-propionic acid methyl ester (1.05 g, 3.17 mmoles) from Example 14B in dioxane (10 mL) was treated with 5N potassium hydroxide (10 mL) and stirred for 4 hours at 60° C. The dioxane was removed under reduced pressure, the residue was neutralized with 1N HCl to pH=7 and extracted three times with 4:1 THF:DCM. The combined organic extracts were dried (MgSO₄), filtered and the filtrate concentrated under reduced pressure to provide the title compound as a white solid (0.9 g, 90%).

### EXAMPLE 14D

A solution of 2-methyl-2-[4-(5-trifluoromethyl-pyridin-2-yl)-piperazin-1-yl]-propionic acid (159 mg, 0.5 mmoles) from Example 14C in DCM (5 mL) and DIPEA (0.5 mL) was treated with hydroxybenzotriazole hydrate (HOBt) (84 mg, 0.6 mmoles), 5-hydroxy-2-adamantamine (100 mg, 0.6 mmoles) from Example 13A and 15 minutes later with (3-dimethylaminopropyl)-3-ethylcarbodiimide HCl (EDCI) (115 mg, 0.6 mmoles). The mixture was stirred overnight at room temperature after which the DCM was removed under reduced pressure and the residue was partitioned between water and ethyl acetate. The aqueous layer was extracted with ethyl acetate and the combined organic extracts washed with saturated sodium bicarbonate, water, dried (MgSO₄) and filtered. The filtrate was concentrated under reduced pressure and the crude product purified (silica gel, 10-40% acetone in hexane) to provide the title compound as a white solid (160 mg, 69%). ¹H NMR (300 MHz, CDCl₃) δ 8.41 (s, 1H), 7.67 (m, 2H), 6.66 (d, J=9.1 Hz, 1H), 4.0 (d, J=7.8 Hz, 1H), 3.66 (m, 4H), 2.64 (m, 4H), 2.23-2.1 (m, 3H), 1.9-1.63 (m, 10H), 1.25 (s, 6H); MS(APCI+) m/z 467 (M+H)⁺.

### EXAMPLE 15

### E-4-{2-Methyl-2-[4-(5-trifluoromethyl-pyridin-2-yl)-piperazin-1-yl]-propionylamino}-adamantane-1-carboxylic acid

### EXAMPLE 15A 2-Adamantanone-5-carboxylic acid methyl ester

A solution of 5-hydroxy-2-adamantanone (2.0 g, 12.0 mmol) in 99% formic acid (12 mL) was added dropwise with vigorous gas evolution over 40 minutes to a rapidly stirred 30% oleum solution (48 mL) heated to 60° C. (W. J. le Noble, S. Srivastava, C. K. Cheung, J. Org. Chem. 48: 1099-1101, 1983). Upon completion of addition, more 99% formic acid (12 mL) was slowly added over the next 40 minutes. The mixture was stirred another 60 minutes at 60° C. and then slowly poured into vigorously stirred methanol (100 mL) cooled to 0° C. The mixture was allowed to slowly warm to 23° C. while stirring for 2 hours and then concentrated in vacuo. The residue was poured onto ice (30 g) and methylene chloride (100 mL) added. The layers were separated, and the aqueous phase extracted twice more with methylene chloride (100 mL aliquots). The combined methylene chloride solutions were concentrated in vacuo to 50 mL, washed with brine, dried over Na₂SO₄, filtered, and concentrated in vacuo to provide the title compound as a pale yellow solid (2.5 g, 99% crude). ¹H NMR (300 MHz, DMSO-d₆) δ 3.61 (s, 3H), 2.47-2.40 (bs, 2H), 2.17-1.96 (m, 9H), 1.93-1.82 (m, 2H); MS (DCI) m/z 209 (M+H)⁺.

### EXAMPLE 15B

### E- and Z-4-Adamantamine-1-carboxylic acid methyl ester

A solution of 2-adamantanone-5-carboxylic acid methyl ester (2.0 g, 9.6 mmoles) from Example 15A and 4 Å molecular sieves (1.0 g) in methanolic ammonia (7N, 17 mL) was stirred overnight at room temperature. The reaction mixture was cooled in an ice bath, treated portionwise with sodium borohydride (1.46 g, 38.4 mmoles) and stirred at room temperature for 2 hours. The suspension was filtered and MeOH was removed under reduced pressure. The residue was taken into methylene chloride (200 mL) and acidified with 10% citric acid. The pH of the solution was adjusted to neutral with saturated NaHCO₃ and then saturated with NaCl. The layers were separated and the aqueous extracted twice more with methylene chloride. The combined organic extracts were dried over Na₂SO₄ and filtered. The filtrate was concentrated under reduced pressure to provide the title compound as a light yellow solid (1.7 g, 85% crude). ¹H NMR (300 MHz, CDCl₃) δ 3.66 (s, 3H), 3.16 (m, 1H), 2.27-1.46 (m, 13H); MS (DCI) m/z 210 (M+H)⁺.

### EXAMPLE 15C

### E- and Z-4-{2-Methyl-2-[4-(5-trifluoromethyl-pyridin-2-yl)-piperazin-1-yl]-propionylamino}-adamantane-1-carboxylic acid methyl ester

To a 0° C., heterogeneous solution of 2-methyl-2-[4-(5-trifluoromethyl-pyridin-2-yl)-piperazin-1-yl]-propionic acid (50 mg, 0.16 mmol) from Example 14C, E- and Z-4-adamantamine-1-carboxylic acid methyl ester (33 mg, 0.16 mmol) from Example 15B, tetrahydrofuran (1.3 mL), and Hunig's base (30 mg, 0.24 mmol) was added solid HATU (60 mg, 0.16 mmol). The stirred reaction mixture was allowed to slowly warm to 23° C. as the ice bath melted overnight (16 hours). LC/MS analysis of the homogenous reaction mixture revealed complete consumption of starting materials. The reaction mixture was concentrated under reduced pressure, and the residue purified with flash silica gel (ethyl acetate/hexanes, 20-80% gradient) to afford the title compound as a mixture of E/Z structural isomers (30 mg, 37%). Carried on as a slightly impure E/Z mixture.

### EXAMPLE 15D

### E-4-{2-Methyl-2-[4-(5-trifluoromethyl-pyridin-2-yl)-piperazin-1-yl]-propionylamino}-adamantane-1-carboxylic acid

A stirred, 23° C., homogenous solution of E- and Z-4-{2-methyl-2-[4-(5-trifluoromethyl-pyridin-2-yl)-piperazin-1-yl]-propionylamino}-adamantane-1-carboxylic acid methyl ester (19 mg, 0.037 mmol) from Example 15C and methanol (0.5 mL) became cloudy upon addition of 10% aqueous NaOH (1 mL). After stirring for 1 hour at 23° C., the reaction mixture was heated to 50° C. for 1 hour. The mixture was diluted with sat. aqueous NaHCO₃ and extracted three times with a tetrahydrofuran/methylene chloride solution (4/1). The combined organic extracts were dried over Na₂SO₄, filtered, and concentrated under reduced pressure. The E/Z isomers were separated by radial chromatography with 2% methanol in ethyl acetate/hexanes (4/1) as the eluant to afford the title compound (5 mg, 27%). ¹H NMR (500 MHz, DMSO-d₆) δ 8.41 (s, 1H), 7.79 (dd, J=2.5, 9 Hz, 1H), 7.71 (d, J=7.5 Hz, 1H), 6.96 (d, J=9.5 Hz, 1H), 3.79 (m, 1H), 3.66 (m, 4H), 2.54 (m, 4H), 1.95-1.70 (m, 11H), 1.58-1.52 (m, 2H), 1.13 (s, 6H); MS (DCI) m/z 495 (M+H)⁺.

### EXAMPLE 16

### E-4-({1-[4-(5-Trifluoromethyl-pyridin-2-yl)-piperazin-1-yl]-cyclopropanecarbonyl}-amino)-adamantane-1-carboxylic acid

### EXAMPLE 16A

### N,N-Bis-(2-hydroxy-ethyl)-2-nitrobenzenesulfonamide

A solution of 2-nitrobenzenesulfonyl chloride (10.5 g, 47.6 mmol) in anhydrous methylene chloride (25 mL) was added dropwise with stirring to a 0° C. solution of diethanolamine (5.00 g, 47.6 mmol) and triethylamine (4.92 g, 47.6 mmol) in anhydrous methylene chloride (50 mL). Reaction stirred three hours at 0° C. and then overnight at room temperature. Reaction mixture concentrated under reduced pressure. Residue dissolved in ethyl acetate, washed with 1 N NaOH, saturated NaHCO₃, and brine, dried over Na₂SO₄, filtered, and concentrated under reduced pressure. The residue was purified by normal phase HPLC on a Biotage pre-packed silica gel column eluting with ethyl acetate to afford the title compound (7.00 g, 51%). MS (ESI) m/z 291 (M+H)⁺.

### EXAMPLE 16B

### N,N-Bis-(2-trifluoromethanesulfonyloxyethyl)-2-nitrobenzenesulfonamide

Triflic anhydride (13.6 g, 48.3 mmol) was added dropwise with stirring to a 0° C. solution of N,N-bis-(2-hydroxyethyl)-2-nitrobenzenesulfonamide (7.00 g, 24.1 mmol) from Example 16A and 2,4,6-collidine (5.85 g, 48.3 mmol) in anhydrous methylene chloride (50 mL) (J. A. Kozlowski, et al., Bioorg. Med. Chem. Lett. 12: 791-794, 2002). Reaction stirred two hours at 0° C. and then overnight at room temperature. Reaction diluted with chloroform, washed with saturated NaHCO₃ and brine, dried over Na₂SO₄, filtered, and concentrated under reduced pressure. The residue was purified by normal phase HPLC on a Biotage pre-packed silica gel column eluting with 3:1 hexane:ethyl acetate to afford the title compound. MS (ESI) m/z 555 (M+H)⁺.

### EXAMPLE 16C

### 1-[4-(2-Nitrobenzenesulfonyl)-piperazin-1-yl]-cyclopropanecarboxylic acid methyl ester

A solution of N,N-bis-(2-trifluoromethanesulfonyloxyethyl)-2-nitrobenzenesulfonamide (1.83 g, 3.30 mmol) from Example 16B and 1-aminocyclopropane-1-carboxylic acid methyl ester HCl (0.50 g, 3.30 mmol) in anhydrous acetonitrile (10 mL) was treated with sodium carbonate (1.40 g, 13.2 mmol) and heated overnight at 60° C. (J. A. Kozlowski, et al., Bioorg. Med. Chem. Lett. 12: 791-794, 2002). Reaction diluted with ethyl acetate, washed with water and brine, dried over Na₂SO₄, filtered, and concentrated under reduced pressure. The residue was purified by normal phase HPLC on a Biotage pre-packed silica gel column eluting with 3:1 hexane:ethyl acetate to afford the title compound (1.78 g, 80%). MS (ESI) m/z 370 (M+H)⁺.

### EXAMPLE 16D

### 1-[4-(5-Trifluoromethylpyridin-2-yl)-piperazin-1-yl]-cyclopropanecarboxylic acid methyl ester

A solution of 1-[4-(2-nitrobenzenesulfonyl)-piperazin-1-yl]-cyclopropanecarboxylic acid methyl ester (0.60 g, 1.63 mmol) from Example 16C in anhydrous dimethylformamide (5 mL) was treated with potassium carbonate (0.67 g, 4.88 mmol) and thiophenol (0.21 g, 1.95 mmol) and stirred one hour at room temperature. This reaction mixture was then treated with 2-bromo-5-trifluoromethylpyridine (0.44 g, 1.95 mmol) and heated overnight at 80° C. Reaction diluted with ethyl acetate, washed with water and brine, dried over Na₂SO₄, filtered, and concentrated under reduced pressure. The residue was purified by normal phase HPLC on a Biotage pre-packed silica gel column eluting with 9:1 hexane:ethyl acetate to afford the title compound (0.32 g, 59%). MS (ESI) m/z 330 (M+H)⁺.

### EXAMPLE 16E

### 1-[4-(5-Trifluoromethylpyridin-2-yl)-piperazin-1-yl]-cyclopropanecarboxylic acid

A solution of 1-[4-(5-trifluoromethylpyridin-2-yl)-piperazin-1-yl]-cyclopropanecarboxylic acid methyl ester (0.32 g, 0.96 mmol) from Example 16D in tetrahydrofuran (5 mL) and methanol (2 mL) was treated with 4 N sodium hydroxide (2.40 mL, 9.60 mmol) and stirred overnight at 60° C. Reaction mixture concentrated under reduced pressure and dissolved in water. Solution neutralized with 1 N phosphoric acid (pH 7) and extracted three times with chloroform. Extracts dried over Na₂SO₄, filtered, and concentrated under reduced pressure to afford the title compound without further purification. MS (ESI) m/z 316 (M+H)⁺.

### EXAMPLE 16F

### E- and Z-4-({1-[4-(5-Trifluoromethyl-pyridin-2-yl)-piperazin-1-yl]-cyclopropanecarbonyl}-amino)-adamantane-1-carboxylic acid methyl ester

A solution of 1-[4-(5-trifluoromethylpyridin-2-yl)-piperazin-1-yl]-cyclopropanecarboxylic acid (60 mg, 0.19 mmol) from Example 16E, E- and Z-4-adamantamine-1-carboxylic acid methyl ester (40 mg, 0.19 mmol) from Example 15B, and O-(1H-benzotriazol-1-yl)-N,N,N′,N′-tetramethyluronium tetrafluoroborate (TBTU) (92 mg, 0.29 mmol) in dimethylformamide (3 mL) was treated, after stirring 5 minutes at room temperature, with N,N-diisopropylethylamine (50 mg, 0.38 mmol) and stirred overnight at room temperature. Reaction diluted with ethyl acetate, washed with water, saturated NaHCO₃, and brine, dried over Na₂SO₄, filtered, and concentrated under reduced pressure. The residue was purified by flash chromatography on silica gel eluting with 8:2 to 6:4 hexane:ethyl acetate to afford the title compound (72 mg, 75%). MS (ESI) m/z 507 (M+H)⁺.

### EXAMPLE 16G

### E-4-({1-[4-(5-Trifluoromethyl-pyridin-2-yl)-piperazin-1-yl]-cyclopropanecarbonyl}-amino)-adamantane-1-carboxylic acid

The title compound was prepared using the procedure described in Example 16E starting with E- and Z-4-({1-[4-(5-trifluoromethyl-pyridin-2-yl)-piperazin-1-yl]-cyclopropanecarbonyl}-amino)-adamantane-1-carboxylic acid methyl ester from Example 16F. The E and Z isomers were separated by flash chromatography on silica gel eluting with 20:1 to 10:1 methylene chloride:methanol to afford the title compound (37 mg, 53%). ¹H NMR (400 MHz, DMSO-d₆) δ 8.41 (s, 1H), 8.23 (d, J=7.5 Hz, 1H), 7.79 (dd, J=2.5, 9 Hz, 1H), 6.96 (d, J=9.5 Hz, 1H), 3.79 (m, 1H), 3.70 (m, 4H), 2.50 (m, 4H), 2.00-1.70 (m, 11H), 1.60-1.52 (m, 2H), 1.05 (m, 2H), 0.96 (m, 2H); MS (ESI) m/z 493 (M+H)⁺.

### EXAMPLE 17

### E-4-({1-[4-(5-Trifluoromethyl-pyridin-2-yl)-piperazin-1-yl]-cyclopropanecarbonyl}-amino)-adamantane-1-carboxylic acid amide

The title compound was prepared according to the procedure outlined in Example 23 substituting E-4-({1-[4-(5-trifluoromethyl-pyridin-2-yl)-piperazin-1-yl]-cyclopropanecarbonyl}-amino)-adamantane-1-carboxylic acid from example 16G for E-4-{2-methyl-2-[4-(5-trifluoromethyl-pyridin-2-yl)-piperazin-1-yl]-propionylamino}-adamantane-1-carboxylic acid. ¹H NMR (500 MHz, DMSO-d₆) δ 8.47 (s, 1H), 8.31 (d, J=9.5 Hz, 1H), 7.86 (dd, J=2.5, 9 Hz, 1H), 7.03 (d, J=9.5 Hz, 2H), 6.75 (bs, 1H), 3.88 (m, 1H), 3.77 (m, 4H), 2.57 (m, 4H), 2.05-1.80 (m, 11H), 1.61 (m, 2H), 1.12 (m, 2H), 1.03 (m, 2H); MS (ESI) m/z 492 (M+H)⁺.

### EXAMPLE 18

### E-4-{2-[4-(5-Trifluoromethyl-pyridin-2-yl)-piperazin-1-yl]-butyrylamino}-adamantane-1-carboxylic acid amide

### EXAMPLE 18A

### E- and Z-4-Formylamino-adamantane-1-carboxylic acid methyl ester

A solution of E- and Z-4-adamantamine-1-carboxylic acid methyl ester (12.7 g, 60.2 mmol) from Example 15B in methyl formate (60 mL) was treated with triethylamine (12.2 g, 120 mmol) and heated overnight at 50° C. in a high pressure tube. The reaction mixture was concentrated under reduced pressure. The residue was purified by normal phase HPLC on a Biotage pre-packed silica gel column eluting with 7:3 ethyl acetate:hexane to afford the title compound (6.00 g, 42%). MS (DCI) m/z 238 (M+H)⁺.

### EXAMPLE 18B

### E-4-Isocyano-adamantane-1-carboxylic acid methyl ester

A-10° C. solution of E- and Z-4-formylamino-adamantane-1-carboxylic acid methyl ester (6.00 g, 25.3 mmol) from Example 18A and triethylamine (12.8 g, 127 mmol) in anhydrous methylene chloride (30 mL) was treated dropwise with phosphorus oxychloride (5.82 g, 38.0 mmol) and reaction stirred one hour at −10° C. and then one hour at room temperature. Reaction cooled back down to 0° C. and quenched with saturated sodium bicarbonate. Organic layer separated and aqueous layer extracted two times with methylene chloride. Combined extracts dried over Na₂SO₄, filtered, and concentrated under reduced pressure. The E and Z isomers were separated by flash chromatography on silica gel eluting methylene chloride to provide the title compound (1.60 g, 29%). MS (DCI) m/z 220 (M+H)⁺.

### EXAMPLE 18C

### E-4-{2-[4-(5-Trifluoromethyl-pyridin-2-yl)-piperazin-1-yl]-butyrylamino}-adamantane-1-carboxylic acid methyl ester

A heterogeneous solution of 1-[5-trifluoromethyl)-2-pyridyl]piperazine (106 mg, 0.46 mmol), propionaldehyde (14 mg, 0.23 mmol), acetic acid (27 mg, 0.46 mmol), and dried 4 Å molecular sieves (25 mg) in anhydrous methanol (2 mL) which had been stirring at room temperature for twenty minutes was treated with E-4-isocyano-adamantane-1-carboxylic acid methyl ester (50 mg, 0.23 mmol) from Example 18B and stirred two hours at room temperature and overnight at 70° C. The reaction mixture was concentrated under reduced pressure. The residue was purified by flash chromatography on silica gel eluting with 7:3 to 1:1 hexane:ethyl acetate to provide the title compound. MS (ESI) m/z 509 (M+H)⁺.

### EXAMPLE 18D

### E-4-{2-[4-(5-Trifluoromethyl-pyridin-2-yl)-piperazin-1-yl]-butyrylamino}-adamantane-1-carboxylic acid

The title compound was prepared using the procedure described in Example 16E starting with E-4-{2-[4-(5-trifluoromethyl-pyridin-2-yl)-piperazin-1-yl]-butyrylamino}-adamantane-1-carboxylic acid methyl ester from Example 18C. MS (ESI) m/z 495 (M+H)⁺.

### EXAMPLE 18E

### E-4-{2-[4-(5-Trifluoromethyl-pyridin-2-yl)-piperazin-1-yl]-butyrylamino}-adamantane-1-carboxylic acid amide

The title compound was prepared according to the procedure outlined in Example 23 substituting E-4-{2-[4-(5-trifluoromethyl-pyridin-2-yl)-piperazin-1-yl]-butyrylamino}-adamantane-1-carboxylic acid from example 18D for E-4-{2-methyl-2-[4-(5-trifluoromethyl-pyridin-2-yl)-piperazin-1-yl]-propionylamino}-adamantane-1-carboxylic acid. ¹H NMR (500 MHz, DMSO-d₆) δ 8.39 (s, 1H), 7.77 (dd, J=2.5, 9 Hz, 1H), 7.68 (d, J=9.5 Hz, 1H), 6.97 (s, 1H), 6.94 (d, J=9.5 Hz, 1H), 6.71 (s, 1H), 3.82 (m, 1H), 3.58 (m, 4H), 3.12 (m, 1H), 2.65 (m, 2H), 2.56 (m, 2H), 1.95-1.70 (m, 11H), 1.65 (m, 1H), 1.55 (m, 1H), 1.41 (m, 2H), 0.83 (m, 3H); MS (ESI) m/z 494 (M+H)⁺.

### EXAMPLE 19

### E-4-{2-Cyclopropyl-2-[4-(5-trifluoromethyl-pyridin-2-yl)-piperazin-1-yl]-acetylamino}-adamantane-1-carboxylic acid amide

### EXAMPLE 19A

### E-4-{2-Cyclopropyl-2-[4-(5-trifluoromethyl-pyridin-2-yl)-piperazin-1-yl]-acetylamino}-adamantane-1-carboxylic acid

The title compound was prepared using the procedures described in Examples 18C-D substituting cyclopropanecarboxaldehyde for propionaldehyde.

### EXAMPLE 19B

### E-4-{2-Cyclopropyl-2-[4-(5-trifluoromethyl-pyridin-2-yl)-piperazin-1-yl]-acetylamino}-adamantane-1-carboxylic acid amide

The title compound was prepared using the procedures described in Examples 23 substituting E-4-{2-cyclopropyl-2-[4-(5-trifluoromethyl-pyridin-2-yl)-piperazin-1-yl]-acetylamino}-adamantane-1-carboxylic acid from example 19A for E-4-{2-methyl-2-[4-(5-trifluoromethyl-pyridin-2-yl)-piperazin-1-yl]-propionylamino}-adamantane-1-carboxylic acid. ¹H NMR (500 MHz, DMSO-d₆) δ 8.39 (s, 1H), 7.78 (dd, J=2.5, 9 Hz, 1H), 7.56 (d, J=9.5 Hz, 1H), 6.98 (s, 1H), 6.93 (d, J=9.5 Hz, 1H), 6.72 (s, 1H), 3.82 (m, 1H), 3.62 (m, 4H), 2.79 (m, 2H), 2.53 (m, 2H), 2.22 (d, J=9.5 Hz, 1H), 1.95-1.70 (m, 11H), 1.43 (m, 2H), 0.99 (m, 1H), 0.60 (m, 1H), 0.41 (m, 1H), 0.27 (m, 2H); MS (ESI) m/z 506 (M+H)⁺.

### EXAMPLE 20

### E-4-({1-[4-(5-Trifluoromethyl-pyridin-2-yl)-piperazin-1-yl]-cyclobutanecarbonyl}-amino)-adamantane-1-carboxylic acid amide

### EXAMPLE 20A

### E-4-({1-[4-(5-Trifluoromethyl-pyridin-2-yl)-piperazin-1-yl]-cyclobutanecarbonyl}-amino)-adamantane-1-carboxylic acid

The title compound was prepared using the procedures described in Examples 18C-D substituting cyclobutanone for propionaldehyde.

### EXAMPLE 20B

### E-4-({1-[4-(5-Trifluoromethyl-pyridin-2-yl)-piperazin-1-yl]-cyclobutanecarbonyl}-amino)-adamantane-1-carboxylic acid amide

The title compound was prepared using the procedures described in Examples 23 substituting E-4-({1-[4-(5-trifluoromethyl-pyridin-2-yl)-piperazin-1-yl]-cyclobutanecarbonyl}-amino)-adamantane-1-carboxylic acid for E-4-{2-methyl-2-[4-(5-trifluoromethyl-pyridin-2-yl)-piperazin-1-yl]-propionylamino}-adamantane-1-carboxylic acid. ¹H NMR (500 MHz, DMSO-d₆) δ 8.41 (s, 1H), 7.80 (dd, J=2.5, 9 Hz, 1H), 7.36 (d, J=9.5 Hz, 1H), 6.99 (s, 1H), 6.97 (d, J=9.5 Hz, 1H), 6.73 (s, 1H), 3.82 (m, 1H), 3.63 (m, 4H), 2.53 (m, 4H), 2.22 (m, 2H), 2.14 (m, 2H), 1.95-1.60 (m, 13H), 1.46 (m, 2H); MS (ESI) m/z 506 (M+H)⁺.

### EXAMPLE 21

### E-N-(5-Hydroxymethyl-adamantan-2-yl)-2-[4-(5-trifluoromethyl-pyridin-2-yl)-piperazin-1-yl]-isobutyramide

A solution of E-4-{2-methyl-2-[4-(5-trifluoromethyl-pyridin-2-yl)-piperazin-1-yl]-propionylamino}-adamantane-1-carboxylic acid (494 mg, 1 mmoles) in THF (2 mL) was cooled to 0° C. and treated with 1N borane solution in THF (2 mL). The reaction was stirred at reflux for 20 hours and carefully quenched with water (4 mL) after cooling to room temperature. The reaction mixture extracted three times with a tetrahydrofuran/methylene chloride solution (4/1). The combined organic extracts were dried over Na₂SO₄, filtered, and concentrated under reduced pressure. The residue was purified with flash silica gel (acetone/hexanes, 10-40% gradient) to provide the title compound as a white solid (400 mg, 83%). ¹H NMR (300 MHz, CDCl₃) δ 8.41 (s, 1H), 7.77 (d, J=11.5 Hz, 1H), 7.64 (d, J=6.3 Hz, 1H), 6.66 (d, J=9.1 Hz, 1H), 6.76 (s, 1H), 3.96 (bd, 1H), 3.66 (s, 4H), 3.25 (d, J=5.4 Hz, 2H), 2.65 (s, 4H), 1.99 (s, 2H), 1.71-1.56 (m, 12H), 1.25 (s, 6H); MS (ESI+) m/z 481 (M+H)⁺.

### EXAMPLE 22

### E-N-(5-Formyl-adamantan-2-yl)-2-[4-(5-trifluoromethyl-pyridin-2-yl)-piperazin-1-yl]-isobutyramide

A solution of E-N-(5-Hydroxymethyl-adamantan-2-yl)-2-[4-(5-trifluoromethyl-pyridin-2-yl)-piperazin-1-yl]-isobutyramide (400 mg, 0.83 mmoles) from Example 21 and 4 Å molecular sieves in DCE (3 mL) were treated with 4-methylmorpholine-N-oxide (124 mg, 1.24 mmoles) and tetrapropylammonium perruthenate (15 mg, 0.04 mmoles). The reaction was stirred at room temperature for 20 hours, filtered and washed with DCM. DCM was concentrated under reduced pressure to afford the title compound as a white solid (350 mg, 88%).

### EXAMPLE 23

### E-4-{2-Methyl-2-[4-(5-trifluoromethyl-pyridin-2-yl)-piperazin-1-yl]-propionylamino}-adamantane-1-carboxylic acid amide

A solution of E-4-{2-methyl-2-[4-(5-trifluoromethyl-pyridin-2-yl)-piperazin-1-yl]-propionylamino}-adamantane-1-carboxylic acid (100 mg, 0.21 mmoles) from Example 15 in DCM (2 mL) was treated with HOBt (33 mg, 0.22 mmoles) and EDC (46 mg, 0.24 mmoles) and stirred at room temperature for 1 hour. Excess of aqueous (35%) ammonia (2 mL) was added and the reaction was stirred for additional 20 hours. The layers were separated and the aqueous extracted twice more with methylene chloride (2×2 mL). The combined organic extracts were dried over Na₂SO₄ and filtered. The filtrate was concentrated under reduced pressure to provide the crude title compound that was purified on reverse phase HPLC to provide the title compound (80 mg, 81%). ¹H NMR (400 MHz, DMSO-d₆) δ 8.47 (s, 1H), 7.83 (d, J=6.8 Hz, 1H), 7.76 (d, J=7.9 Hz, 1H), 7.02 (d, J=9.5 Hz, 2H), 6.76 (s, 1H), 3.86 (d, J=7.9 Hz, 1H), 3.71 (s, 4H), 2.59 (s, 4H), 1.98-1.90 (m, 7H), 1.81-1.77 (m, 4H), 1.58 (d, J=12.9 Hz, 2H), 1.18 (s, 6H); MS (ESI+) m/z 494 (M+H)⁺.

### EXAMPLE 24

### E-4-{2-Methyl-2-[4-(5-trifluoromethyl-pyridin-2-yl)-piperazin-1-yl]-propionylamino}-adamantane-1-carboxylic acid hydroxyamide

A solution of E-4-{2-methyl-2-[4-(5-trifluoromethyl-pyridin-2-yl)-piperazin-1-yl]-propionylamino}-adamantane-1-carboxylic acid (100 mg, 0.21 mmoles) from Example 15 in DCM (2 mL) was treated with HOBt (33 mg, 0.22 mmoles) and EDC (46 mg, 0.24 mmoles) and stirred at room temperature for 1 hour. Excess of aqueous hydroxylamine (2 mL) was added and the reaction was stirred for additional 20 hours. The layers were separated and the aqueous extracted twice more with methylene chloride (2×2 mL). The combined organic extracts were dried over Na₂SO₄ and filtered. The filtrate was concentrated under reduced pressure to provide the crude title compound that was purified on reverse phase HPLC to provide the title compound (20 mg, 19%). ¹H NMR (400 MHz, Py-d₅) δ 8.67 (s, 1H), 7.85 (d, J=8.3 Hz, 1H), 7.79 (d, J=9.2 Hz, 1H), 6.86 (d, J=8.9 Hz, 1H), 4.3 (d, J=8.3 Hz, 1H), 3.74 (s, 4H), 2.57 (s, 4H), 2.29 (s, 4H), 2.18 (s, 2H), 2.11 (s, 2H), 1.97 (s, 1H), 1.86 (d, J=13.5 Hz, 2H)), 1.62 (d, J=13.3 Hz, 2H), 1.31 (s, 6H); MS (ESI+) m/z 510 (M+H)⁺.

### EXAMPLE 25

### E-4-{2-[4-(5-Trifluormethyl-pyridin-2-yl)-piperazin-1-yl]-acetylamino}-adamantane-1-carboxylic acid

### EXAMPLE 25A

### E- and Z-2-Chloro-N-(5-hydroxy-adamantan-2-yl)-acetamide

A solution of E- and Z-5-hydroxy-2-adamantamine (1.7 g, 10 mmoles) in DCM (33 mL) and DIPEA (1.47 g, 11.4 mmoles) was cooled in an ice bath and treated with 2-chloroacetyl chloride (0.88 mL, 11 mmoles). The mixture was stirred for 2.5 hours at room temperature and DCM was removed under reduced pressure. The residue was partitioned between water and ethyl acetate. The organic layer was washed with saturated 1 N HCl, water, brine, dried (Na₂SO₄), filtered, and concentrated in vacuo. The isomers were separated by column chromatography (silica gel, 10-30% acetone in hexane) to furnish 0.6 g of E-2-chloro-N-(5-hydroxy-adamantan-2-yl)acetamide and 0.27 g of Z-2-chloro-N-(5-hydroxy-adamantan-2-yl)acetamide.

### EXAMPLE 25B

### E-4-(2-Chloro-acetylamino)-adamantane-1-carboxylic acid methyl ester

A solution of E-2-chloro-N-(5-hydroxy-adamantan-2-yl)-2-methyl-acetamide (0.5 g, 2.1 mmol) from Example 25A in 99% formic acid (3 mL) was added dropwise by addition funnel with vigorous gas evolution to a rapidly stirred 30% oleum solution (13 mL) heated to 60° C. (W. J. le Noble, S. Srivastava, C. K. Cheung, J. Org. Chem. 48: 1099-1101, 1983). Upon completion of addition, more 99% formic acid (3 mL) was slowly added by addition funnel. The mixture was stirred another 60 minutes at 60° C. and then slowly poured into vigorously stirred ice water. The mixture was allowed to slowly warm to 23° C., filtered and washed with water to neutral pH. The precipitate was dried in a vacuum oven, taken into MeOH (3 mL) and treated with thionyl chloride at 0° C. (0.25 mL, 3.5 mmoles). The reaction mixture was stirring at room temperature for 3 hours and then MeOH was evaporated under reduced pressure to provide the title compound as an off-white solid (0.5 g, 85% crude).

### EXAMPLE 25C

### E-4-{2-[4-(5-Trifluormethyl-pyridin-2-yl)-piperazin-1-yl]-acetylamino}-adamantane-1-carboxylic acid

A solution of E-4-(2-chloro-acetylamino)-adamantane-1-carboxylic acid methyl ester (0.075 g, 0.26 mmoles) from Example 25B, in MeOH (1.5 mL) and DIPEA (0.05 mL, 0.29 mmoles) was treated with 1-(5-trifluoromethyl-pyridin-2-yl)-piperazine (0.091 g, 0.39 mmoles) and stirred for 2 hours at 80° C. The cooled reaction mixture was purified on reverse phase HPLC and hydrolyzed with 3N HCl at 60° C. over 6 hours. Drying of the reaction mixture under reduced pressure provided the title compound as a white solid (50 mg, 40%). ¹H NMR (300 MHz, DMSO-d₆) δ 10.48 (bs, 1H), 8.56 (d, J=7.2 Hz, 1H), 8.48 (bs, 1H), 7.92 (dd, J=2.4, 9.0 Hz, 1H), 7.07 (d, J=9.0 Hz, 1H), 4.51 (m, 2H), 4.06 (s, 2H), 3.89 (m, 1H), 3.56 (m, 2H), 3.41 (m, 2H), 3.21 (bs, 2H), 1.90 (m, 9H), 1.80 (m, 2H), 1.47 (m, 2H); MS (DCI+) m/z 467 (M+H)⁺.

### EXAMPLE 26

### E-4-[2-(3,3-Difluoro-piperidin-1-yl)-acetylamino]-adamantane-1-carboxylic acid

A solution of E-4-(2-chloro-acetylamino)-adamantane-1-carboxylic acid methyl ester (0.075 g, 0.26 mmoles) from Example 25B, in MeOH (1.5 mL) and DIPEA (0.05 mL, 0.29 mmoles) was treated with 3,3-difluoro-piperidine hydrochloride (0.062 g, 0.39 mmoles) and stirred for 2 hours at 80° C. The cooled reaction mixture was purified on reverse phase HPLC and hydrolyzed with 3N HCl at 60° C. over 6 hours. Drying of the reaction mixture under reduced pressure provided the hydrochloride salt of the title compound as a white solid (50 mg, 52%). ¹H NMR (300 MHz, DMSO-d₆) δ 8.45 (m, 1H), 3.97 (bs, 2H), 3.88 (m, 1H), 3.65 (m, 2H), 3.23 (m, 2H), 2.11 (m, 2H), 1.91 (m, 11H), 1.79 (m, 2H), 1.47 (m, 2H); MS (DCI+) m/z 357 (M+H)⁺.

### EXAMPLE 27

### E-4-[2-(2-Trifluoromethyl-pyrrolidin-1-yl)-acetylamino]-adamantane-1-carboxylic acid

A solution of E-4-(2-chloro-acetylamino)-adamantane-1-carboxylic acid methyl ester (0.075 g, 0.26 mmoles) from Example 25B, in MeOH (1.5 mL) and DIPEA (0.05 mL, 0.29 mmoles) was treated with 2-trifluoromethylpyrrolidine (0.055 g, 0.39 mmoles) and stirred for 2 hours at 80° C. The cooled reaction mixture was purified on reverse phase HPLC and hydrolyzed with 3N HCl at 60° C. over 6 hours. Drying of the reaction mixture under reduced pressure provided the hydrochloride salt of the title compound as a white solid (50 mg, 49%). ¹H NMR (300 MHz, DMSO-d₆) δ 7.72 (d, J=7.8 Hz, 1H), 3.79 (m, 2H), 3.54 (d, J=16.5 Hz, 1H), 3.36 (d, J=16.5 Hz, 1H), 3.07 (m, 1H), 2.72 (m, 1H), 2.10 (m, 1H), 1.82 (m, 14H), 1.48 (m, 2H); MS (DCI+) m/z 375 (M+H)⁺.

### EXAMPLE 28

### E-4-{2-[4-(5-Trifluoromethyl-pyridin-2-yl)-piperazin-1-yl]-acetylamino}-adamantane-1-carboxylic acid amide

A solution of E-4-{2-[4-(5-trifluoromethyl-pyridin-2-yl)-piperazin-1-yl]-acetylamino}-adamantane-1-carboxylic acid (100 mg, 0.21 mmoles) from Example 25C in DCM (2 mL) was treated with HOBt (32 mg, 0.21 mmoles) and EDC (46 mg, 0.24 mmoles) and stirred at room temperature for 1 hour. Excess of aqueous (35%) ammonia (2 mL) was added and the reaction was stirred for additional 20 hours. The layers were separated and the aqueous extracted twice more with methylene chloride (2×2 mL). The combined organic extracts were dried over Na₂SO₄ and filtered. The filtrate was concentrated under reduced pressure to provide the crude title compound that was purified on reverse phase HPLC to afford the title compound (75 mg, 75%). ¹H NMR (400 MHz, Py-d₅) δ 8.64 (s, 1H), 7.9 (d, J=7.6 Hz, 1H), 7.77 (d, J=9.2 Hz, 1H), 6.82 (d, J=9.2 Hz, 1H), 4.39 (d, J=8.3 Hz, 1H), 3.72 (t, J=4.9 Hz, 4H), 3.25 (s, 2H), 2.62 (t, J=4.9 Hz, 4H), 2.26 (m, 4H), 2.17 (s, 4H), 1.96 (m, 3H), 1.6 (d J=12.6 Hz, 2H); MS (ESI+) m/z 466 (M+H)⁺.

### EXAMPLE 29

### E-4-[2-(2-Trifluoromethyl-pyrrolidin-1-yl)-acetylamino]-adamantane-1-carboxylic acid amide

A solution of E-4-[2-(2-trifluoromethyl-pyrrolidin-1-yl)-acetylamino]-adamantane-1-carboxylic acid (74 mg, 0.2 mmoles) from Example 27 in DCM (2 mL) was treated with HOBt (33 mg, 0.22 mmoles) and EDC (46 mg, 0.24 mmoles) and stirred at room temperature for 1 hour. Excess of aqueous (35%) ammonia (2 mL) was added and the reaction was stirred for additional 20 hours. The layers were separated and the aqueous extracted twice more with methylene chloride (2×2 mL). The combined organic extracts were dried over Na₂SO₄ and filtered. The filtrate was concentrated under reduced pressure to provide the crude title compound which was purified on reverse phase HPLC to afford the title compound (45 mg, 60%). ¹H NMR (300 MHz, CDCl₃) δ 7.6 (d, J=6.4 Hz, 1H), 5.57-5.2 (bd, 2H), 4.05 (d, J=8.1 Hz, 1H), 3.56 (d, J=17 Hz, 1H), 3.32 (m, 2H), 3.22 (m, 1H), 2.58 (q, J=7.4 Hz, 1H), 2.08-1.90 (m, 13H), 1.77 (m, 2H), 1.65 (m, 2H); MS (ESI+) m/z 374 (M+H)⁺.

### EXAMPLE 30

### E-4-[2-(3,3-Difluoro-piperidin-1-yl)-acetylamino]-adamantane-1-carboxylic acid amide

A solution of E-4-[2-(3,3-difluoro-piperidin-1-yl)-acetylamino]-adamantane-1-carboxylic acid (71 mg, 0.2 mmoles) from Example 26 in DCM (2 mL) was treated with HOBt (33 mg, 0.22 mmoles) and EDC (46 mg, 0.24 mmoles) and stirred at room temperature for 1 hour. Excess of aqueous (35%) ammonia (2 mL) was added and the reaction was stirred for additional 20 hours. The layers were separated and the aqueous extracted twice more with methylene chloride. The combined organic extracts were dried over Na₂SO₄ and filtered. The filtrate was concentrated under reduced pressure to provide the crude title compound which was purified on reverse phase HPLC to afford the title compound (40 mg, 56%). ¹H NMR (300 MHz, CDCl₃) δ 7.74 (d, J=8.5 Hz, 1H), 5.54-5.18 (bd, 2H), 4.06 (d, J=8.5 Hz, 1H), 3.12 (s, 2H), 2.78 (t, J=11.2 Hz, 2H), 2.62 (bs, 2H), 2.08-1.80 (m, 15H), 1.6 (m, 2H); MS (ESI+) m/z 356 (M+H)⁺.

### EXAMPLE 31

### E-4-[2-(3-Fluoropyrrolidin-1-yl)-propionylamino]-adamantane-1-carboxylic acid amide

### EXAMPLE 31A

### E-4-(2-Bromo-propionylamino)-adamantane-1-carboxylic acid

A solution of E-2-bromo-N-(5-hydroxy-adamantan-2-yl)-propionamide from Example 13B (4.0 g, 13.25 mmol) in 99% formic acid (13 mL) was added dropwise with vigorous gas evolution over 40 minutes to a rapidly stirred 30% oleum solution (40 mL) heated to 60° C. (W. J. le Noble, S. Srivastava, C. K. Cheung, J. Org. Chem. 48: 1099-1101, 1983). Upon completion of addition, more 99% formic acid (13 mL) was slowly added over the next 40 minutes. The mixture was stirred another 60 minutes at 60° C. and then slowly poured into vigorously stirred iced water (100 mL) cooled to 0° C. The mixture was allowed to slowly warm to 23° C. while stirring, filtered and washed with water to neutral pH (1L). The precipitate was dried in a vacuum oven to provide the title compound as a white solid (4.3 g, 99% crude).

### EXAMPLE 311B

### E-4-(2-Bromo-propionylamino)-adamantane-1-carboxylic acid amide

A solution of E-4-(2-bromo-propionylamino)-adamantane-1-carboxylic acid (330 mg, 1 mmol) from Example 31A in DCM (5 mL) was treated with HOBt (168 mg, 1.1 mmol) and EDC (230 mg, 1.2 mmoles) and stirred at room temperature for 1 hour. Excess of aqueous (35%) ammonia (5 mL) was added and the reaction was stirred for additional 2 hours. The layers were separated and the aqueous extracted twice more with methylene chloride (2×5 mL). The combined organic extracts were dried over Na₂SO₄ and filtered. The filtrate was concentrated under reduced pressure. The residue was taken into MeOH and formed a white precipitate that was filtered to provide the title compound as a white solid (210 mg, 64%).

### EXAMPLE 31C

### E-4-[2-(3-Fluoropyrrolidin-1-yl)-propionylamino]-adamantane-1-carboxylic acid amide

A solution of E-4-(2-bromo-propionylamino)-adamantane-1-carboxylic acid amide (33 mg, 0.1 mmol) from Example 31B and the hydrochloride of (−)-3R-fluoropyrrolidine (15 mg, 0.12 mmol) in MeOH (0.5 mL) and DIPEA (0.1 mL) was stirred overnight at 70° C. The MeOH was removed under reduced pressure and the residue purified on reverse phase HPLC to provide the title compound as a mixture of 2 diastereomers (20 mg, 59%). ¹H NMR (400 MHz, Py-d₅) δ 7.7 (two d, 1H), 5.2-5.08 (bd, 2H), 4.32 (m, 1H), 3.56 (s, 4H), 3.29-2.95 (m, 2H), 2.6-2.5 (m, 2H), 2.25-2.0 (m, 10H), 1.95 (m, 3H), 1.37 (two d, 3H), 1.4 (t, 2H); MS (ESI+) m/z 338 (M+H)⁺.

### EXAMPLE 32

### E-4-[2-(3,3-Difluoropiperidine-1-yl)-propionylamino]-adamantane-1-carboxylic acid amide

A solution of E-4-(2-bromo-propionylamino)-adamantane-1-carboxylic acid amide (33 mg, 0.1 mmoles) and the hydrochloride of 3,3-difluoropiperidine (19 mg, 0.12 mmol) from Example 31B in MeOH (0.5 mL) and DIPEA (0.1 mL) was stirred overnight at 70° C. The MeOH was removed under reduced pressure and the residue purified on reverse phase HPLC to provide the title compound as a white solid (18 mg, 48%). ¹H NMR (400 MHz, Py-d₅) δ 7.92 (d, J=7.7 Hz, 1H), 7.51 (s, 2H), 4.32 (d, J=7.7 Hz, 1H), 3.42 (q, J=7 Hz, 1H), 2.92 (q, J=10.7 Hz, 1H), 2.78 (q, J=11.6 Hz, 1H), 2.5 (m, 2H), 2.27-2.10 (m, 8H), 1.98-1.88 (m, 5H), 1.68 (m, 2H), 1.55 (m, 2H), 1.32 (d, 3H); MS (ESI+) m/z 370 (M+H)⁺.

### EXAMPLE 33

### E-4-[2-(2-Trifluoromethylpyrrolidin-1-yl)-propionylamino]-adamantane-1-carboxylic acid amide

A solution of E-4-(2-Bromo-propionylamino)-adamantane-1-carboxylic acid amide (33 mg, 0.1 mmol) from Example 31B and the hydrochloride of 2-trifluoromethylpyrrolidine (21 mg, 0.12 mmol) in MeOH (0.5 mL) and DIPEA (0.1 mL) was stirred overnight at 70° C. The MeOH was removed under reduced pressure and the residue purified on reverse phase HPLC to provide the title compound as a mixture of 4 diastereomers (20 mg, 51%). ¹H NMR (400 MHz, Py-d₅) δ 7.81 (d, 1H), 4.32 (two d, 1H), 3.8 (two m, 2H), 3.2 (two m, 1H), 2.7 (two m, 1H), 2.48-1.5 (m, 17H), 1.47 (two d, 3H); MS (ESI+) m/z 388 (M+H)⁺.

### EXAMPLE 34

### E-4-{2-[4-(5-Chloro-pyridin-2-yl)-piperazin-1-yl]-2-methyl-propionylamino}-adamantane-1-carboxylic acid

### EXAMPLE 34A

### 2-Bromo-N-(5-hydroxy-adamantan-2-yl)-2-methyl-propionamide

A solution of E- and Z-5-hydroxy-2-adamantamine (8.7 g, 52 mmol) from Example 13A in DCM (150 mL) and DIPEA (25 mL) was cooled in an ice bath and treated with 2-bromoisobutyryl bromide (7.2 mL, 58 mmol) in DCM (25 mL). The mixture was stirred for 2 hours at room temperature and DCM was removed under reduced pressure. The residue was partitioned between water and ethyl acetate. The organic layer was washed with saturated sodium bicarbonate, water, dried (MgSO₄) and filtered. The filtrate was concentrated under reduced pressure to provide the title compound as a dark beige solid (11.7 g, 71%). The isomers were separated by column chromatography (silica gel, 5-35% acetone in hexane) to furnish 7.8 g of E-2-bromo-N-(5-hydroxy-adamantan-2-yl)-2-methyl-propionamide and 3.9 g of Z-2-bromo-N-(5-hydroxy-adamantan-2-yl)-2-methyl-propionamide.

### EXAMPLE 34B

### E-4-(2-Bromo-2-methyl-propionylamino)-adamantane-1-carboxylic acid methyl ester

A solution of E-2-bromo-N-(5-hydroxy-adamantan-2-yl)-2-methyl-propionamide (7.84 g, 24.8 mmol) from Example 34A in 99% formic acid (25 mL) was added dropwise with vigorous gas evolution over 40 minutes to a rapidly stirred 30% oleum solution (75 mL) heated to 60° C. (W. J. le Noble, S. Srivastava, C. K. Cheung, J. Org. Chem. 48: 1099-1101, 1983). Upon completion of addition, more 99% formic acid (25 mL) was slowly added over the next 40 minutes. The mixture was stirred another 60 minutes at 60° C. and then slowly poured into vigorously stirred iced water (300 mL) cooled to 0° C. The mixture was allowed to slowly warm to 23° C., filtered and washed with water to neutral pH (1L). The precipitate was dried in a vacuum oven, taken into MeOH and treated with thionyl chloride at 0° C. (2 mL, 28 mmol). The reaction mixture was stirring at room temperature for 3 hours and then MeOH was evaporated under reduced pressure to provide the title compound as an off-white solid (8.7 g, 97% crude).

### EXAMPLE 34C

### E-4-{2-[4-(5-Chloro-pyridin-2-yl)-piperazin-1-yl]-2-methyl-propionylamino}-adamantane-1-carboxylic acid

A two phase suspension of E-4-(2-bromo-2-methyl-propionylamino)-adamantane-1-carboxylic acid methyl ester (36 mg, 0.1 mmol) from Example 34B, 1-(5-chloro-2-pyridyl)piperazine (20 mg, 0.11 mmol) and tetrabutylammonium bromide (3 mg, 0.01 mmol) in DCM (0.2 mL) and 50% NaOH (0.2 mL) was stirred at room temperature for 20 hours. After that the reaction mixture was diluted with water and DCM and layers separated. Organic layer was washed with water (2×2 mL), dried (MgSO₄) and filtered. The filtrate was concentrated under reduced pressure to provide crude methyl ester of the title compound that was purified on reverse phase HPLC and hydrolyzed with 3N HCL at 60° C. over 6 hours. Drying of the reaction mixture under reduced pressure provided the title compound as a white solid (35 mg, 75%). ¹H NMR (400 MHz, Py-d₅) δ 8.38 (s, 1H), 7.87 (d, J=7.8 Hz, 1H), 6.8 (d, J=9 Hz, 1H), 4.31 (d, J=8.1 Hz, 1H), 3.64 (s, 4H), 2.59 (s, 4H), 2.25 (m, 4H), 2.17 (s, 2H), 2.11 (s, 2H), 1.96 (s, 1H), 1.87 (d, J=14.4 Hz, 2H), 1.62 (d, J=12.8 Hz, 2H), 1.31 (s, 6H); MS (ESI+) m/z 461 (M+H)⁺.

### EXAMPLE 35

### E-4-[2-Methyl-2-(1,2,4,5-tetrahydro-benzo[d]azepin-3-yl)-propionylamino]-adamantane-1-carboxylic acid

The title compound was prepared according to the procedure outlined in Example 34C substituting 2,3,4,5-tetrahydro-1H-benzo[d]azepine for 1-(5-chloro-2-pyridyl)piperazine (23 mg, 57%). ¹H NMR (400 MHz, Py-d₅) δ 7.85 (d, J=7.8 Hz, 1H), 7.24 (m, 4H), 4.33 (d, J=7.5 Hz, 1H), 2.9 (m, 4H), 2.56 (s, 4H), 2.32 (q, J=14 Hz, 4H), 2.22 (s, 1H), 2.16 (s, 1H), 2.01 (s, 1H), 1.88 (d, J=12.8 Hz, 2H), 1.78 (m, 2H), 1.65 (d, J=13.4 Hz, 2H), 1.28 (s, 6H); MS (ESI+) m/z 411 (M+H)⁺.

### EXAMPLE 36

### E-4-[2-Methyl-2-(4-m-tolyl-[1,4]diazepan-1-yl)-propionylamino]-adamantane-1-carboxylic acid

The title compound was prepared according to the procedure outlined in Example 34C substituting 1-m-tolyl-[1,4]diazepane for 1-(5-chloro-2-pyridyl)piperazine (23 mg, 51%). ¹H NMR (400 MHz, Py-d₅) δ 7.27 (t, J=7.7 Hz, 1H), 6.74 (s, 1H), 6.69 (d, J=6.4 Hz, 1H), 6.65 (d, J=8.6 Hz, 1H), 4.3 (d, J=7.3 Hz, 1H), 3.54 (t, J=8 Hz, 2H), 2.8 (s, 1H), 2.5 (s, 1H), 2.3 (s, 3H), 2.25 (m, 5H), 2.16 (m, 5H), 1.93 (m, 3H), 1.79 (m, 2H), 1.58 (m, 2H), 1.31 (s, 6H), 1.27 (t, J=7.4 Hz, 2H); MS(ESI+) m/z 454 (M+H)⁺.

### EXAMPLE 37

### E-4-[2-Methyl-2-(4-phenyl-piperidin-1-yl)-propionylamino]-adamantane-1-carboxylic acid

The title compound was prepared according to the method of procedure outlined in Example 34C substituting 4-phenyl-piperidine for 1-(5-chloro-2-pyridyl)piperazine (21 mg, 50%). ¹H NMR (400 MHz, Py-d₅) δ 7.96 (d, J=8.1 Hz, 1H), 7,41 (m, 4H), 7.29 (m, 1H), 4.3 (d, J=8.1 Hz, 1H), 2.93 (d, J=11.6 Hz, 2H), 2.53 (m, 1H), 2.31-2.12 (m, 10H), 1.90 (m, 5H), 1.77 (m, 2H), 1.6 (d, J=12.8 Hz, 2H), 1.35 (s, 6H); MS(ESI+) m/z 425 (M+H)⁺.

### EXAMPLE 38

### E-4-{2-[4-(4-Chloro-phenyl)-piperidin-1-yl]-2-methyl-propionylamino}-adamantane-1-carboxylic acid

The title compound was prepared according to the procedure outlined in Example 34C substituting 4-(4-chloro-phenyl)-piperidine for 1-(5-chloro-2-pyridyl)piperazine (24 mg, 56%). ¹H NMR (400 MHz, Py-d₅) δ 7.92 (d, J=8.1 Hz, 1H), 7,42 (d, J=8.5 Hz, 2H), 7.29 (d, J=8.7 Hz, 2H), 4.3 (d, J=8.1 Hz, 1H), 2.93 (d, J=11.6 Hz, 2H), 2.48 (m, 1H), 2.31-2.12 (m, 10H), 1.90 (m, 5H), 1.77 (m, 2H), 1.6 (d, J=13.1 Hz, 2H), 1.35 (s, 6H); MS(ESI+) m/z 459 (M+H)⁺.

### EXAMPLE 39

### E-4-{2-[5-(6-Chloro-pyridin-3-yl)-hexahydro-pyrrolo[3,4-c]pyrrol-2-yl]-2-methyl-propionylamino}-adamantane-1-carboxylic acid amide

### EXAMPLE 39A

### E-4-{2-[5-(6-Chloro-pyridin-3-yl)-hexahydro-pyrrolo[3,4-c]pyrrol-2-yl]-2-methyl-propionylamino}-adamantane-1-carboxylic acid

The title compound was prepared according to the procedure outlined in Example 34C substituting 2,3,4,5-tetrahydro-1H-benzo[d]azepine for 1-(5-chloro-2-pyridyl)piperazine (23 mg, 48%).

### EXAMPLE 39B

### E-4-{2-[5-(6-Chloro-pyridin-3-yl)-hexahydro-pyrrolo[3,4-c]pyrrol-2-yl]-2-methyl-propionylamino}-adamantane-1-carboxylic acid amide

The title compound was prepared according to the procedure outlined in Example 23 substituting E-4-{2-[5-(6-chloro-pyridin-3-yl)-hexahydro-pyrrolo[3,4-c]pyrrol-2-yl]-2-methyl-propionylamino}-adamantane-1-carboxylic acid for E-4-{2-methyl-2-[4-(5-trifluoromethyl-pyridin-2-yl)-piperazin-1-yl]-propionylamino}-adamantane-1-carboxylic acid (16 mg, 70%). ¹H NMR (400 MHz, Py-d₅) δ 7.98 (d, J=3.1 Hz, 1H), 7.73 (d, J=8.1 Hz, 1H), 7.32 (d, J=8.6 Hz, 1H), 6.98 (m, 1H), 4.23 (d, J=8.1 Hz, 1H), 3.32 (m, 2H), 3.12 (m, 2H), 2.76 (s, 2H), 2.59 (m, 4H), 2.16 (m, 4H), 2.01 (s, 4H), 1.6 (m, 3H), 1.38 (m, 2H), 1.31 (s, 6H); MS (ESI+) m/z 486 (M+H)⁺.

### EXAMPLE 40

### E-4-{2-[4-(5-Fluoro-pyridin-3-yl)-[1,4]diazepan-1-yl]-2-methyl-propionylamino}-adamantane-1-carboxylic acid amide

### EXAMPLE 40A

### E-4-{2-[4-(5-Fluoro-pyridin-3-yl)-[1,4]diazepan-1-yl]-2-methyl-propionylamino}-adamantane-1-carboxylic acid

The title compound was prepared according to the procedure outlined in Example 34C substituting 1-(5-fluoro-pyridin-3-yl)-[1,4]diazepane for 1-(5-chloro-2-pyridyl)piperazine (20 mg, 43%).

### EXAMPLE 40B

### E-4-{2-[4-(5-Fluoro-pyridin-3-yl)-[1,4]diazepan-1-yl]-2-methyl-propionylamino}-adamantane-1-carboxylic acid amide

The title compound was prepared according to the procedure outlined in Example 23 substituting E-4-{2-[4-(5-fluoro-pyridin-3-yl)-[1,4]diazepan-1-yl]-2-methyl-propionylamino}-adamantane-1-carboxylic acid for E-4-{2-methyl-2-[4-(5-trifluoromethyl-pyridin-2-yl)-piperazin-1-yl]-propionylamino}-adamantane-1-carboxylic acid (15 mg, 75%). ¹H NMR (400 MHz, Py-d₅) δ 8.28 (s, 1H), 8.13 (s, 1H), 7.44 (d, J=8 Hz, 1H), 7.0 (d, J=8 Hz, 1H), 4.25 (d, J=8.1 Hz, 1H), 3.5 (m, 4H), 2.73 (s, 2H), 2.45 (s, 2H), 2.23 (m, 4H), 2.14 (s, 2H), 2.06 (s, 2H), 1.9 (s, 1H), 1.79 (m, 2H), 1.66 (d, J=12.8 Hz, 2H), 1.55 (d, J=12.8 Hz, 2H), 1.29 (s, 6H); MS (ESI+) m/z 458 (M+H)⁺.

### EXAMPLE 41

### E-4-[2-Methyl-2-(3-pyridin-3-yl-3,9-diaza-bicyclo[4.2.1]non-9-yl)-propionylamino]-adamantane-1-carboxylic acid amide

### EXAMPLE 41A

### E-4-[2-Methyl-2-(3-pyridin-3-yl-3,9-diaza-bicyclo[4.2.1]non-9-yl)-propionylamino]-adamantane-1-carboxylic acid

The title compound was prepared according to the procedure outline in Example 34C substituting 3-pyridin-3-yl-3,9-diaza-bicyclo[4.2.1]nonane for 1-(5-chloro-2-pyridyl)piperazine (25 mg, 53%).

### EXAMPLE 41B

### E-4-[2-Methyl-2-(3-pyridin-3-yl-3,9-diaza-bicyclo[4.2.1]non-9-yl)-propionylamino]-adamantane-1-carboxylic acid amide

The title compound was prepared according to the procedure outlined in Example 23 substituting E-4-[2-methyl-2-(3-pyridin-3-yl-3,9-diaza-bicyclo[4.2.1]non-9-yl)-propionylamino]-adamantane-1-carboxylic acid for E-4-{2-methyl-2-[4-(5-trifluoromethyl-pyridin-2-yl)-piperazin-1-yl]-propionylamino}-adamantane-1-carboxylic acid (16 mg, 64%). ¹H NMR (300 MHz, CDCl₃) δ 7.84 (s, 1H), 3.99 (d, J=8.1 Hz, 1H), 3.35 (d, J=5.9 Hz, 1H), 2.71-2.65 (bd, 4H), 2.16-2.10 (m, 3H), 1.89 (d, J=11.9 Hz, 2H), 1.77-1.65 (m, 14H), 1.52 (d, J=12.8 Hz, 2H), 1.24 (d, J=6.9 Hz, 3H); MS(ESI+) m/z 466 (M+H)⁺.

### EXAMPLE 42

### E-4-[2-Methyl-2-(2-trifluoromethyl-pyrrolidin-1-yl)-propionylamino]-adamantane-1-carboxylic acid amide

### EXAMPLE 42A

### E-4-[2-Methyl-2-(2-trifluoromethyl-pyrrolidin-1-yl)-propionylamino]-adamantane-1-carboxylic acid

The title compound was prepared according to the procedure outlined in Example 34C substituting 2-trifluoromethylpyrrolidine for 1-(5-chloro-2-pyridyl)piperazine (19 mg, 47%).

### EXAMPLE 42B

### E-4-[2-Methyl-2-(2-trifluoromethyl-pyrrolidin-1-yl)-propionylamino]-adamantane-1-carboxylic acid amide

The title compound was prepared according to the procedure outlined in Example 23 substituting E-4-[2-methyl-2-(2-trifluoromethyl-pyrrolidin-1-yl)-propionylamino]-adamantane-1-carboxylic acid for E-4-{2-methyl-2-[4-(5-trifluoromethyl-pyridin-2-yl)-piperazin-1-yl]-propionylamino}-adamantane-1-carboxylic acid (12 mg, 63%). ¹H NMR (400 MHz, Py-d₅) δ 7.43 (d, J=7.8 Hz, 1H), 5.54 (bs, 1H), 5.18 (bs, 1H), 3.99 (d, J=8.1 Hz, 1H), 3.68 (m, 1H), 3.05 (m, 1H), 2.82 (m, 1H), 2.05-1.9 (m, 12H), 1.77 (d, J=13.1 Hz, 3H), 1.65 (m, 2H), 1.35 (s, 3H); 1.21 (s, 3H); MS(ESI+) m/z 402 (M+H)⁺.

### EXAMPLE 43

### E-4-[2-(3,3-Difluoro-piperidin-1-yl)-2-methyl-propionylamino]-adamantane-1-carboxylic acid amide

### EXAMPLE 43A

### E-4-[2-(3,3-Difluoro-piperidin-1-yl)-2-methyl-propionylamino]-adamantane-1-carboxylic acid

The title compound was prepared according to the procedure outlined in Example 34C substituting 3,3-difluoropiperidine for 1-(5-chloro-2-pyridyl)piperazine (19 mg, 47%).

### EXAMPLE 43B

### E-4-[2-(3,3-Difluoro-piperidin-1-yl)-2-methyl-propionylamino]-adamantane-1-carboxylic acid amide

The title compound was prepared according to the procedure outlined in Example 23 substituting E-4-[2-(3,3-difluoro-piperidin-1-yl)-2-methyl-propionylamino]-adamantane-1-carboxylic acid for E-4-{2-methyl-2-[4-(5-trifluoromethyl-pyridin-2-yl)-piperazin-1-yl]-propionylamino}-adamantane-1-carboxylic acid (12 mg, 66%). ¹H NMR (400 MHz, Py-d₅) δ 7.71 (s, 1H), 5.55 (bs, 1H), 5.22 (bs, 1H), 3.96 (d, J=8.1 Hz, 1H), 2.71 (s, 2H), 2.54 (s, 2H), 2.05-1.9 (m, 11H), 1.8 (m, 4H), 1.6 (d, J=13.1 Hz, 2H), 1.23 (s, 6H); MS(ESI+) m/z 384 (M+H)⁺.

### EXAMPLE 44

### E-4-[2-(3-Fluoro-pyrrolidin-1-yl)-2-methyl-propionylamino]-adamantane-1-carboxylic acid amide

### EXAMPLE 44A

### E-4-(2-Bromo-2-methyl-propionylamino)-adamantane-1-carboxylic acid

A solution of E-2-bromo-N-(5-hydroxy-adamantan-2-yl)-2-methyl-propionamide (7.84 g, 24.8 mmol) from Example 34A in 99% formic acid (25 mL) was added dropwise with vigorous gas evolution over 40 minutes to a rapidly stirred 30% oleum solution (75 mL) heated to 60° C. (W. J. le Noble, S. Srivastava, C. K. Cheung, J. Org. Chem. 48: 1099-1101, 1983). Upon completion of addition, more 99% formic acid (25 mL) was slowly added over the next 40 minutes. The mixture was stirred another 60 minutes at 60° C. and then slowly poured into vigorously stirred iced water (300 mL) cooled to 0° C. The mixture was allowed to slowly warm to 23° C., filtered and washed with water to neutral pH (1L). The precipitate was dried in a vacuum oven, to provide the title compound as an white solid (8.1 g, 95%).

### EXAMPLE 44B

### E-4-(2-Bromo-2-methyl-propionylamino)-adamantane-1-carboxylic acid amide

A solution of (1.72 g, 5 mmol) in E-4-(2-bromo-2-methyl-propionylamino)-adamantane-1-carboxylic acid from Example 44A in DCM (15 mL) was treated with HOBt (841 mg, 1.1 mmol) and EDC (1.15 g, 6 mmol) and stirred at room temperature for 1 hour. Excess of aqueous (35%) ammonia (15 mL) was added and the reaction was stirred for additional 2 hours. The layers were separated and the aqueous extracted twice more with methylene chloride (2×1 5 mL). The combined organic extracts were dried over Na₂SO₄ and filtered. The filtrate was concentrated under reduced pressure. The residue was taken into MeOH and formed a white precipitate that was filtered to provide the title compound as a white solid (1.1 g, 64%)

### EXAMPLE 44C

### E-4-[2-(3-Fluoro-pyrrolidin-1-yl)-2-methyl-propionylamino]-adamantane-1-carboxylic acid amide

A two phase suspension of E-4-(2-bromo-2-methyl-propionylamino)-adamantane-1-carboxylic acid amide (35 mg, 0.1 mmol) from Example 44B, 3-R-fluoropyrrolidine (14 mg, 0.11 mmol) and tetrabutylammonium bromide (3 mg, 0.01 mmol) in DCM (0.2 mL) and 50% NaOH (0.2 mL) was stirred at room temperature for 20 hours. After that the reaction mixture was diluted with water and DCM and layers separated. Organic layer was washed with water (2×2 mL), dried (MgSO₄) and filtered. The filtrate was concentrated under reduced pressure to provide the title compound as a white solid (15 mg, 43%). ¹H NMR (300 MHz, Py-d₅) δ 7.91 (d, J=7.7 Hz, 1H), 5.19-5.06 (bd, 1H), 4.29 (d, J=8.0 Hz, 1H), 3.0 (m, 1H), 2.91 (m, 1H), 2.58 (m, 1H), 2.39 (q, J=7.6 Hz, 1H), 2.27-2.01 (m, 7H), 1.96-1.85 (m, 6H), 1.53 (m, 3H), 1.35 (d, 6H); MS (ESI+) m/z 352 (M+H)⁺.

### EXAMPLE 45

### E-4-{2-[4-(5-Trifluormethyl-pyridin-2-yl)-piperazin-1-yl]-acetylamino}-adamantane-1-carboxamide

A solution of E-4-(2-bromo-propionylamino)-adamantane-1-carboxylic acid amide (0.075 g, 0.23 mmol) from Example 31B in MeOH (1.0 mL) and DIPEA (0.044 mL, 0.25 mmol) was treated with 1-(5-trifluoromethyl-pyridin-2-yl)-piperazine (0.058 g, 0.25 mmol) and stirred for 48 hours at 70° C. The cooled reaction mixture was purified on reverse phase HPLC and drying of the reaction mixture under reduced pressure provided the TFA salt of the title compound as a white solid (60 mg, 44%). ¹H NMR (400 MHz, Py-d₅) δ 8.66 (s, 1H), 7.93 (d, J=8 Hz, 1H), 7.77 (dd, J=2.8, 9.2 Hz, 1H), 7.62 (s, 1H), 6.84 (d, J=8.8 Hz, 1H), 4.36 (m, 1H), 3.74 (m, 4H), 3.33 (q, J=6.8 Hz, 1H), 2.67 (m, 2H), 2.57 (m, 2H), 2.27 (m, 4H), 2.16 (m, 5H), 1.94 (m, 3H), 1.60 (m, 2H), 1.34 (d, J=6.8 Hz, 3H); MS (DCI+) m/z 480 (M+H)⁺.

### EXAMPLE 46

### E-4-[2-(3,3-Difluoro-piperidin-1-yl)-2-methyl-propionylamino]-adamantane-1-carboxylic acid 3,4-dimethoxy-benzylamide

A solution of Example 43A (35.0 mg, 0.09 mmol) in DMA (5 mL) was treated with TBTU (O-(Benzotrialzol-1-yl)-1,1,3,3-tetramethyluronium tetrafluoroborate) (43.3 mg, 0.135 mmol), 3,4-dimethoxy-benzylamine (18.0 mg, 0.108 mmol) and DIEA (Ethyl-diisopropyl-amine) (0.033 ml, 0.18 mmol). The mixture was stirred at room temperature for 12 hours. The reaction mixture was concentrated under reduced pressure. The residue was purified by RP-HPLC to provide the title compound (8 mg, 16%). ¹H NMR (400 MHz, DMSO-D6) δ ppm 1.12 (s, 6H) 1.49-1.58 (m, 2H) 1.64-1.74 (m, 4H) 1.77-1.84 (m, 2H) 1.84-2.00 (m, 9H) 2.43-2.49 (m, 2H) 2.69 (m, 2H) 3.72 (s, 3H) 3.73 (s, 3H) 3.79 (m, 1H) 4.19 (d, J=5.83 Hz, 2H) 6.72 (dd, J=7.98 Hz, 1.53 Hz, 1H) 6.81 (d, J=1.53 Hz, 1H) 6.87 (d, J=7.98 Hz, 1H) 7.59 (d, J=7.98 Hz, 1H) 7.94 (t, J=5.83 Hz, 1H); MS (ESI+) m/z 534 (M+H)⁺.

### EXAMPLE 47

### E-4-[({4-[2-(3,3-Difluoro-piperidin-1-yl)-2-methyl-propionylamino]-adamantane-1-carbonyl}-amino)-methyl]-benzoic acid

A solution of Example 43A (71.0 mg, 0.18 mmol) in DMF (8 mL) was treated with TBTU (O-(Benzotrialzol-1-yl)-1,1,3,3-tetramethyluronium tetrafluoroborate) (77 mg, 0.27 mmol), 4-aminomethyl-benzoic acid methyl ester (36.0 mg, 0.216 mmol) and DIEA (Ethyl-diisopropyl-amine) (0.066 ml, 0.36 mmol). The mixture was stirred at room temperature for 12 hours. Then DCM (15 mL) and H₂O (5 mL) were added to reaction mixture. The layers were separated and the organic phase were dried over Na₂SO₄ and filtered. The filtrate was concentrated under reduced pressure. The residue was purified by RP-HPLC to provide white powder with MS (ESI+) m/z 532. The white powder was dissolved in THF (2 mL). H₂O (2 mL) and LiOH (24 mg, 1 mmol) were added to the THF solution. The reaction mixture was stirred for at room temperature for 12 hours. Then DCM (15 mL) and H₂O (5 mL) were added to reaction mixture. The layers were separated and the organic phase was dried over Na₂SO₄ and filtered. The filtrate was concentrated under reduced pressure. The residue was purified by RP-HPLC to provide the title compound (9 mg, 10%). ¹H NMR (500 MHz, DMSO-D6) δ ppm 1.12 (s, 6H) 1.50-1.59 (m, J=12.79 Hz, 2H) 1.63-1.74 (m, 4H) 1.82 (d, J=2.18 Hz, 2H) 1.85-1.97 (m, 9H) 2.44-2.49 (m, 2H) 2.69 (t, J=11.07 Hz, 2H) 3.78 (d, J=7.49 Hz, 1H) 4.30 (d, J=5.93 Hz, 2H) 7.26 (d, J=8.11 Hz, 2H) 7.59 (d, J=8.11 Hz, 1H) 7.85 (d, J=8.11 Hz, 2H) 8.07 (t, J=5.93 Hz, 1H); MS (ESI+) m/z 518 (M+H)⁺.

### EXAMPLE 48

### E-4-[2-(3,3-Difluoro-piperidin-1-yl)-2-methyl-propionylamino]-adamantane-1-carboxylic acid (furan-2-ylmethyl)-amide

A solution of Example 43A (35.0 mg, 0.09 mmol) in DMF (5 mL) was treated with TBTU (O-(Benzotrialzol-1-yl)-1,1,3,3-tetramethyluronium tetrafluoroborate) (43.3 mg, 0.135 mmol), furfurylamine (10.5 mg, 0.108 mmol) and DIEA (Ethyl-diisopropyl-amine) (0.033 ml, 0.18 mmol). The mixture was stirred at room temperature for 12 hours. The reaction mixture was concentrated under reduced pressure. The residue was purified by RP-HPLC to provide the title compound (6 mg, 14%). ¹H NMR (400 MHz, DMSO-D6) δ ppm 0.85-1.01 (s, 6H) 1.40-1.55 (m, 2H) 1.55-1.79 (m, 19H) 2.24-2.34 (m, 2H) 3.50-3.58 (m, 1H) 6.93-7.01 (m, 3H) 7.07 (t, J=7.67 Hz, 2H) 7.26 (t, J=5.52 Hz, 1H) 7.37 (d, J=7.98 Hz, 1H); MS (ESI+) m/z 464 (M+H)⁺.

### EXAMPLE 49

### E-4-[2-(3,3-Difluoro-piperidin-1-yl)-2-methyl-propionylamino]-adamantane-1-carboxylic acid (thiazol-5-ylmethyl)-amide

A solution of Example 43A (35.0 mg, 0.09 mmol) in DMA (5 mL) was treated with TBTU (O-(Benzotrialzol-1-yl)-1,1,3,3-tetramethyluronium tetrafluoroborate) (43.3 mg, 0.135 mmol), thiazol-5-yl-methylamine (12.0 mg, 0.108 mmol) and DIEA (Ethyl-diisopropyl-amine) (0.033 ml, 0.18 mmol). The mixture was stirred at room temperature for 12 hours. The reaction mixture was concentrated under reduced pressure. The residue was purified by RP-HPLC to provide the title compound (5 mg, 12%). ¹H NMR (400 MHz, DMSO-D6) δ ppm 1.12 (s, 6H) 1.48-1.59 (m, 2H) 1.64-1.76 (m, 4H) 1.80-1.85 (m, 2H) 1.86-2.00 (m, 9H) 2.44-2.49 (m, 2H) 2.69 (t, J=11.51 Hz, 2H) 3.78 (d, J=7.67 Hz, 1H) 4.39 (d, J=6.14 Hz, 2H) 7.26 (s, 1H) 7.59 (d, J=7.67 Hz, 1H) 8.03 (t, J=6.14 Hz, 1H) 9.01-9.05 (m, 1H); MS (ESI+) m/z 481 (M+H)⁺.

### EXAMPLE 50

### E-4-[2-(3,3-Difluoro-piperidin-1-yl)-2-methyl-propionylamino]-adamantane-1-carboxylic acid 2-methoxy-benzylamide

A solution of Example 43A (35.0 mg, 0.09 mmol) in DMA (5 mL) was treated with TBTU (O-(Benzotrialzol-1-yl)-1,1,3,3-tetramethyluronium tetrafluoroborate) (43.3 mg, 0.135 mmol), 2-methoxy-benzylamine (15.0 mg, 0.108 mmol) and DIEA (Ethyl-diisopropyl-amine) (0.033 ml, 0.18 mmol). The mixture was stirred at room temperature for 12 hours. The reaction mixture was concentrated under reduced pressure. The residue was purified by RP-HPLC to provide the title compound (7 mg, 15%). ¹H NMR (400 MHz, DMSO-D6) δ ppm 1.10-1.15 (m, 6H) 1.51-1.99 (m, 17H) 2.44-2.49 (m, 2H) 2.64-2.74 (m, 2H) 3.58-3.60 (m, 1H) 3.80 (s, 3H) 4.22 (d, J=5.83 Hz, 2H) 6.86-6.93 (m, 1H) 6.94-6.98 (m, 1H) 7.02-7.07 (m, 1H) 7.17-7.24 (m, 1H) 7.57-7.63 (m, 1H) 7.79-7.85 (m, 1H); MS (ESI+) m/z 504 (M+H)⁺.

### EXAMPLE 51

### E-4-(2-Methyl-2-phenylamino-propionylamino)-adamantane-1-carboxylic acid amide

E-4-(2-Methyl-2-phenylamino-propionylamino)-adamantane-1-carboxylic acid (MS (ESI+) m/z 357 (M+H)⁺) was prepared according to the method of Example 34 substituting aniline for 1-(5-chloro-2-pyridyl)piperazine. A solution of E-4-(2-Methyl-2-phenylamino-propionylamino)-adamantane-1-carboxylic acid (23.6 mg, 0.07 mmol) in DCM (1 mL) was treated with HOBt (10 mg, 0.073 mmol) and EDC (15.4 mg, 0.08 mmol) and stirred at room temperature for 1 hour. Excess of aqueous (30%) ammonia (1 mL) was added and the reaction was stirred at room temperature for additional 2 hours. The reaction mixture was concentrated under reduced pressure. The residue was purified by RP-HPLC to provide the title compound (12 mg, 51%). ¹H NMR (300 MHz, DMSO-D6) δ ppm 1.24-1.34 (m, 2H) 1.37 (s, 6H) 1.38-1.48 (m, 2H) 1.59-1.89 (m, 9H) 3.78 (d, J=7.80 Hz, 1H) 5.81 (s, 1H) 6.53 (d, 2H) 6.60 (m, 1H) 6.69 (s, 1H) 6.95 (s, 1H) 7.03-7.13 (m, 2H) 7.26 (d, 1H); MS (ESI+) m/z 356 (M+H)⁺.

### EXAMPLE 52

### E-4-[2-Methyl-2-(3-pyridin-3-yl-3,9-diaza-bicyclo[4.2.1]non-9-yl)-propionylamino]-adamantane-1-carboxylic acid amide

### EXAMPLE 52A

### E-4-[2-Methyl-2-(3-pyridin-3-yl-3,9-diaza-bicyclo[4.2.1]non-9-yl)-propionylamino]-adamantane-1-carboxylic acid

The title compound was prepared according to the method outlined in Example 34C substituting 3-pyridin-3-yl-3,9-diaza-bicyclo[4.2.1]nonane for 1-(5-chloro-2-pyridyl)piperazine (25 mg, 53%).

### EXAMPLE 52B

### E-4-[2-Methyl-2-(3-pyridin-3-yl-3,9-diaza-bicyclo[4.2.1]non-9-yl)-propionylamino]-adamantane-1-carboxylic acid amide

The title compound was prepared according to the method outlined in Example 23 substituting E-4-[2-methyl-2-(3-pyridin-3-yl-3,9-diaza-bicyclo[4.2.1]non-9-yl)-propionylamino]-adamantane-1-carboxylic acid for E-4-{2-methyl-2-[4-(5-trifluoromethyl-pyridin-2-yl)-piperazin-1-yl]-propionylamino}-adamantane-1-carboxylic acid (16 mg, 64%). ¹H NMR (400 MHz, Py-d₅) δ 8.56 (d, J=2.4 Hz, 1H), 8.18 (d, J=3 Hz, 1H), 7.32 (d, J=7.7 Hz, 1H), 7.18 (m, 2H), 4.31 (d, J=7.7 Hz, 1H), 3.74 (d, J=13.5 Hz, 1H), 3.56 (m, 2H), 3.40 (m, 2H), 3.1 (d, J=13.5 Hz, 1H), 2.29-2.04 (m, 12H), 1.95-1.85 (m, 2H), 1.7701.74 (m, 2H), 1.57 (m, 2H), 1.4 (m, 1H), 1.31 (s, 6H); MS(ESI+) m/z 466 (M+H)⁺.

### EXAMPLE 53

### E-4-{2-Methyl-2-[5-(3-trifluoromethyl-phenyl)-[1,5]diazocan-1-yl]-propionylamino}-adamantane-1-carboxylic acid

The title compound was prepared according to the method outlined in Example 34C substituting 1-(3-trifluoromethyl-phenyl)-[1,5]diazocane for 1-(5-chloro-2-pyridyl)piperazine (26 mg, 50%). ¹H NMR (400 MHz, Py-d₅) δ 7.42 (t, J=7.8 Hz, 1H), 7.07 (d, J=7.6 Hz, 1H), 7.03 (s, 1H), 6.91 (d, J=8.6 Hz, 1H), 4.25 (s, 1H), 3.55 (s, 4H), 2.53 (s, 4H), 2.26 (m, 4H), 2.16 (s, 4H), 1.94 (m, 2H), 1.76 (s, 5H), 1.58 (m, 2H), 1.33 (s, 6H); MS(ESI+) m/z 522 (M+H)⁺.

### EXAMPLE 54

### E-4-{2-[7-(5-Bromo-pyridin-2-yl)-3,7-diaza-bicyclo[3.3.1]non-3-yl]-2-methyl-propionylamino}-adamantane-1-carboxylic acid amide

### EXAMPLE 54A

### E-4-{2-[7-(5-Bromo-pyridin-2-yl)-3,7-diaza-bicyclo[3.3.1]non-3-yl]-2-methyl-propionylamino}-adamantane-1-carboxylic acid

The title compound was prepared according to the method outlined in Example 34C substituting 3-(5-bromo-pyridin-2-yl)-3,7-diaza-bicyclo[3.3.1]nonane for (25 mg, 46%).

### EXAMPLE 54B

### E-4-{2-[7-(5-Bromo-pyridin-2-yl)-3,7-diaza-bicyclo[3.3.1]non-3-yl]-2-methyl-propionylamino}-adamantane-1-carboxylic acid amide

The title compound was prepared according to the method outlined in Example 23 substituting E-4-{2-[7-(5-bromo-pyridin-2-yl)-3,7 diaza-bicyclo[3.3.1]non-3-yl]-2-methyl-propionylamino}-adamantane-1-carboxylic acid for E-4-{2-methyl-2-[4-(5-trifluoromethyl-pyridin-2-yl)-piperazin-1-yl]-propionylamino}-adamantane-1-carboxylic acid (16 mg, 64%). ¹H NMR (400 MHz, Py-d₅) δ 8.48 (s, 1H), 7.69 (m, 1H), 7.14 (d, J=4.1 Hz, 1H), 6.55 (d, J=9.2 Hz, 1H), 4.03 (d, J=6.1 Hz, 1H), 3.8 (d, J=12.6 Hz, 2H), 3.18 (m, 2H), 2.75 (d, J=11 Hz, 2H), 2.32-2.14 (m, 9H), 2.04-2.0 (m, 4H), 1.69 (s, 1H), 1.5-1.39 (m, 3H), 1.20 (s, 6H), 1.15 (d, J=12.6 Hz, 2H); MS(ESI+) m/z 545 (M+H)⁺.

### Biological Data

Measurement of Inhibition Constants:

The ability of test compounds to inhibit human 11β-HSD-1 enzymatic activity in vitro was evaluated in a Scintillation Proximity Assay (SPA). Tritiated-cortisone substrate, NADPH cofactor and titrated compound were incubated with truncated human 11β-HSD-1 enzyme (24-287AA) at room temperature to allow the conversion to cortisol to occur. The reaction was stopped by adding a non-specific 11β-HSD inhibitor, 18β-glycyrrhetinic acid. The tritiated cortisol was captured by a mixture of an anti-cortisol monoclonal antibody and SPA beads coated with anti-mouse antibodies. The reaction plate was shaken at room temperature and the radioactivity bound to SPA beads was then measured on a β-scintillation counter. The 11-βHSD-1 assay was carried out in 96-well microtiter plates in a total volume of 220 μl. To start the assay, 188 μl of master mix which contained 17.5 nM ³H-cortisone, 157.5 nM cortisone, and 181 mM NADPH was added to the wells. In order to drive the reaction in the forward direction, 1 mM G-6-P was also added. Solid compound was dissolved in DMSO to make a 10 mM stock followed by a subsequent 10-fold dilution with 3% DMSO in Tris/EDTA buffer (pH 7.4). 22 μl of titrated compounds was then added in triplicate to the substrate. Reactions were initiated by the addition of 10 μl of 0.1 mg/ml E. coli lysates overexpressing 11β-HSD-1 enzyme. After shaking and incubating plates for 30 minutes at room temperature, reactions were stopped by adding 10 μl of 1 mM glycyrrhetinic acid. The product, tritiated cortisol, was captured by adding 10 μl of 1 μM monoclonal anti-cortisol antibodies and 100 μl SPA beads coated with anti-mouse antibodies. After shaking for 30 minutes, plates were read on a liquid scintillation counter Topcount. Percent inhibition was calculated based on the background and the maximal signal. Wells that contained substrate without compound or enzyme were used as the background, while the wells that contained substrate and enzyme without any compound were considered as maximal signal. Percent of inhibition of each compound was calculated relative to the maximal signal and IC₅₀ curves were generated. This assay was applied to 11β-HSD-2 as well, whereby tritiated cortisol and NAD⁺ were used as substrate and cofactor, respectively.

Compounds of the present invention are active in the 11-βHSD-1 assay described above, and show selectivity for human 11-β-HSD-1 over human 11-β-HSD-2, as indicated in Table 1.

TABLE 1

| Compound | 11-β-HSD-1 IC₅₀ (nM) | 11-β-HSD-2 IC₅₀ (nM) |
|---|---|---|
| A | 110 | >10,000 |
| B | 92 | >10,000 |
| C | 150 | >10,000 |
| D | 140 | >10,000 |
| E | 82 | >10,000 |

The data in Table 1 indicates that the compounds of the present invention are active in the human 11β-HSD-1 enzymatic SPA assay described above, and show selectivity for 11β-HSD-1 over 11β-HSD-2. The 11β-HSD-1 inhibitors of this invention generally have an inhibition constant IC₅₀ of less than 600 nM, and preferably less than 50 nM. The compounds preferably are selective, having an inhibition constant IC₅₀ against 11β-HSD-2 greater than 1000 nM, and preferably greater than 10,000 nM. Generally, the IC₅₀ ratio for 11β-HSD-2 to 11β-HSD-1 of a compound is at least 10 or greater, and preferably 100 or greater.

Mouse Dehydrocorticosterone Challenge Model

Male CD-1 (18-22 g) mice (Charles River, Madison, Wis.) were group housed and allowed free access to food and water. Mice are brought into a quiet procedure room for acclimation the night before the study. Animals are dosed with vehicle or compound at various times (pretreatment period) before being challenged with 11-dehydrocorticosterone (Steraloids Inc., Newport, R.I.). Thirty minutes after challenge, the mice are euthanized with CO₂ and blood samples (EDTA) are obtained by cardiac puncture and immediately placed on ice. Blood samples were then spun, the plasma was removed, and the samples frozen until further analysis was performed. Corticosterone levels were obtained by ELISA (American Laboratory Prod., Co., Windham, N.H.) or HPLC/mass spectroscopy.

TABLE 2
Plasma corticosterone levels following vehicle, 11
dehydrocorticosterone (11-DHC), or the compound described
in example 3 (followed by 11-DHC) treatment.

| Pretreatment | | | | Compound F | Compound F |
|---|---|---|---|---|---|
| Period | | vehicle | 11-DHC | 30 mpk | 100 mpk |
| 0.5 | hours | 231 ± 51 | 1478 ± 180 | 1297 ± 121 | 742 ± 119 |
| 16 | hours | 151 ± 23 | 1200 ± 86 | 1402 ± 99 | 1422 ± 129 |

ob/ob Mouse Model of Type 2 Diabetes.

Male B6.VLep^{ob(-/-)} (ob/ob) mice and their lean littermates (Jackson Laboratory, Bar Harbor, Me.) were group housed and allowed free access to food (Purina 5015) and water. Mice were 6-7 weeks old at the start of each study. On day 0, animals were weighed and postprandial glucose levels determined (Medisense Precision-X™ glucometer, Abbott Laboratories). Mean postprandial glucose levels did not differ significantly from group to group (n=10) at the start of the studies. Animals were weighed, and postprandial glucose measurements were taken weekly throughout the study. On the last day of the study, 16 hours post dose (unless otherwise noted) the mice were euthanized via CO₂, and blood samples (EDTA) were taken by cardiac puncture and immediately placed on ice. Whole blood measurements for HbA1c were taken with hand held meters (A1c NOW, Metrika Inc., Sunnyvale Calif.). Blood samples were then spun and plasma was removed and frozen until further analysis. The plasma triglyceride levels were determined according to instructions by the manufacturer (Infinity kit, Sigma Diagnostics, St. Louis Mo.).

TABLE 3
Plasma glucose, HbA1c, and triglyceride levels following
three weeks of twice daily dosing with vehicle or
the compound described in Example 3.

| | Control | Compound F | Compound F |
|---|---|---|---|
| | ob/ob | 30 mpk | 100 mpk |
| Glucose mg/dL | 338 ± 13 | 295 ± 31 | 263 ± 21 |
| % HbA1c | 6.9 ± 0.3 | 7.6 ± 0.6 | 6.4 ± 0.5 |
| Triglycerides | 348 ± 31 | 255 ± 22 | 282 ± 36 |
| mg/dL | | | |

The compounds of this invention are selective inhibitors of the 11β-HSD-1 enzyme. Their utility in treating or prophylactically treating type 2 diabetes, high blood pressure, dyslipidemia, obesity, metabolic syndrome, and other diseases and conditions is believed to derive from the biochemical mechanism described below.

Biochemical Mechanism

Glucocorticoids are steroid hormones that play an important role in regulating multiple physiological processes in a wide range of tissues and organs. For example, glucocorticoids are potent regulators of glucose and lipid metabolism. Excess glucocorticoid action may lead to insulin resistance, type 2 diabetes, dyslipidemia, visceral obesity and hypertension. Cortisol is the major active and cortisone is the major inactive form of glucocorticoids in humans, while corticosterone and dehydrocorticosterone are the major active and inactive forms in rodents.

Previously, the main determinants of glucocorticoid action were thought to be the circulating hormone concentration and the density of receptors in the target tissues. Only in the last decade, it was discovered that the tissue glucocorticoid level may also be controlled by 11β-hydroxysteroid dehydrogenases enzymes (11β-HSDs). There are two 11β-HSD isozymes which have different substrate affinities and cofactors. The 11β-hydroxysteroid dehydrogenases type 1 enzyme (11β-HSD-1) is a low affinity enzyme with Kₘ for cortisone in the micromolar range that prefers NADPH/NADP⁺ (nicotinamide adenine dinucleotide) as cofactors. 11β-HSD-1 is widely expressed and particularly high expression levels are found in liver, brain, lung, adipose tissue, and vascular smooth muscle cells. In vitro studies indicate that 11β-HSD-1 is capable of acting both as a reductase and a dehydrogenase. However, many studies have shown that it is a predominant reductase in vivo and in intact cells. It converts inactive 11-ketoglucocorticoids (i.e., cortisone or dehydrocorticosterone) to active 11-hydroxyglucocorticoids (i.e., cortisol or corticosterone), and therefore amplifies the glucocorticoid action in a tissue-specific manner.

With only 20% homology to 11β-HSD-1, the 11β-hydroxysteroid dehydrogenases type 2 enzyme (11βHSD-2) is a NAD⁺-dependent, high affinity dehydrogenase with a Kₘ for cortisol in the nanomolar range. 11β-HSD-2 is found primarily in mineralocorticoid target tissues, such as kidney, colon, and placenta. Glucocorticoid action is mediated by the binding of glucocorticoids to receptors, such as mineralocorticoid receptors and glucocorticoid receptors. Through binding to its receptor, the main mineralocorticoid aldosterone controls the water and salts balance in the body. However, the mineralocorticoid receptors have a high affinity for both cortisol and aldosterone. 11β-HSD-2 converts cortisol to inactive cortisone, therefore preventing the non-selective mineralocorticoid receptors from exposure to high levels of cortisol. Mutations in the gene encoding 11β-HSD-2 cause Apparent Mineralocorticoid Excess Syndrome (AME), which is a congenital syndrome resulting in hypokaleamia and severe hypertension. Patients have elevated cortisol levels in mineralocorticoid target tissues due to reduced 11β-HSD-2 activity. The AME symptoms may also be induced by administration of 11β-HSD-2 inhibitor, glycyrrhetinic acid. The activity of 11β-HSD-2 in placenta is probably important for protecting the fetus from excess exposure to maternal glucocorticoids, which may result in hypertension, glucose intolerance and growth retardation.

Since glucocorticoids are potent regulators of glucose and lipid metabolism, excessive glucocorticoid action may lead to insulin resistance, type 2 diabetes, dyslipidemia, visceral obesity and hypertension. The present invention relates to the administration of a therapeutically effective amount of an 11β-HSD-1 inhibitor for the treatment, control, amelioration, and/or delay of onset of diseases and conditions that are mediated by excess, or uncontrolled, amounts of cortisol and/or other corticosteroids. Inhibition of the 11β-HSD-1 enzyme limits the conversion of inactive cortisone to active cortisol. Cortisol may cause, or contribute to, the symptoms of these diseases and conditions if it is present in excessive amounts.

The compounds of this invention are 11β-HSD-1 selective inhibitors when comparing to 11β-HSD-2. Previous studies (B. R. Walker et al., J. of Clin. Endocrinology and Met., 80: 3155-3159, 1995) have demonstrated that administration of 11β-HSD-1 inhibitors improves insulin sensitivity in humans. However, these studies were carried out using the nonselective 11β-HSD-1 inhibitor carbenoxolone. Inhibition of 11β-HSD-2 by carbenoxolone causes serious side effects, such as hypertension.

Although cortisol is an important and well-recognized anti-inflammatory agent (J. Baxer, Pharmac. Ther., 2:605-659, 1976), if present in large amount, it also has detrimental effects. For example, cortisol antagonizes the insulin effect in liver resulting in reduced insulin sensitivity and increased gluconeogenesis. Therefore, patients who already have impaired glucose tolerance have a greater probability of developing type 2 diabetes in the presence of abnormally high levels of cortisol.

Glucocorticoids may bind to and activate GRs (and possibly mineralocorticoid receptors) to potentiate the vasoconstrictive effects of both catecholamines and angiotensin II (M. Pirpiris et al., Hypertension, 19:567-574, 1992, C. Kornel et al., Steroids, 58: 580-587, 1993, B. R. Walker and B. C. Williams, Clin. Sci. 82:597-605, 1992). The 11-HSD-1 enzyme is present in vascular smooth muscle, which is believed to control the contractile response together with 11β-HSD-2. High levels of cortisol in tissues where the mineralocorticoid receptor is present may lead to hypertension. Therefore, administration of a therapeutic dose of an 11β-HSD-1 inhibitor should be effective in treating or prophylactically treating, controlling, and ameliorating the symptoms of NIDDM. Administration of a therapeutically effective amount of an 11β-HSD-1 inhibitor may actually delay, or prevent the onset of type 2 diabetes.

The effects of elevated levels of cortisol are also observed in patients who have Cushing's syndrome (D. N. Orth, N. Engl. J. Med. 332:791-803, 1995, M. Boscaro, et al., Lancet, 357: 783-791, 2001, X. Bertagna, et al, Cushing's Disease. In: Melmed S., Ed. The Pituitary. 2^{nd} ed. Malden, M A: Blackwell; 592-612, 2002), which is a metabolic disease characterized by high levels of cortisol in the blood stream. Patients with Cushing's syndrome often develop type 2 diabetes, obesity, metabolic syndrome and dyslipidemia.

Abdominal obesity is closely associated with glucose intolerance (C. T. Montaque et al., Diabetes, 49: 883-888, 2000), hyperinsulinemia, hypertriglyceridemia, and other factors of metabolic syndrome (also known as syndrome X), such as high blood pressure, elevated VLDL, and reduced HDL. Thus, administration of an effective amount of an 11β-HSD-1 inhibitor may be useful in the treatment or control of obesity by controlling excess cortisol, independent of its effectiveness in treating or prophylactically treating NIDDM. Long-term treatment with an 11β-HSD-1 inhibitor may also be useful in delaying the onset of obesity, or perhaps preventing it entirely if the patients use an 11β-HSD-1 inhibitor in combination with controlled diet and exercise.

By reducing insulin resistance and maintaining serum glucose at normal concentrations, compounds of this invention may also have utility in the treatment and prevention of the numerous conditions that often accompany type 2 diabetes and insulin resistance, including the metabolic syndrome, obesity, reactive hypoglycemia, and diabetic dyslipidemia.

The following diseases, disorders and conditions are related to type 2 diabetes, and some or all of these may be treated, controlled, in some cases prevented and/or have their onset delayed, by treatment with the compounds of this invention: Hyperglycemia, low glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, pancreatitis, abdominal obesity, neurodegenerative disease, retinopathy, nephropathy, neuropathy, metabolic syndrome and other disorders where insulin resistance is a component.

Evidence in rodents and humans suggests that prolonged elevation of plasma glucocorticoid levels impairs cognitive function that becomes more profound with aging. (See, A. M. Issa et al., J. Neurosci., 10:3247-3254, 1990, S. J. Lupien et. al., Nat. Neurosci., 1:69-73 1998, J. L. Yau et al., Neuroscience, 66: 571-581, 1995). Chronic excessive cortisol levels in the brain may result in neuronal loss and neuronal dysfunction. (See, D. S. Kerr et al., Psychobiology 22: 123-133, 1994, C. Woolley, Brain Res. 531: 225-231, 1990, P. W. Landfield, Science, 272: 1249-1251, 1996). Therefore, administration of a therapeutic dose of an 11β-HSD-1 inhibitor reduces, ameliorates, controls and/or prevents cognitive impairment associated with aging and of neuronal dysfunction.

In Cushing's patients, excess cortisol levels causes hypertension. (See, D. N. Orth, N. Engl. J. Med. 332:791-803, 1995, M. Boscaro, et al., Lancet, 357: 783-791, 2001, X. Bertagna, et al, Cushing's Disease. In: Melmed S., Ed. The Pituitary. 2^{nd} ed. Malden, M A: Blackwell; 592-612, 2002). Since hypertension and dyslipidemia contribute to the development of atherosclerosis, administration of a therapeutically effective amount of an 11β-HSD-1 inhibitor treats, controls, delays the onset of, and/or prevents atherosclerosis.

It has been reported that conversion of dehydrocorticosterone to corticosterone by 11β-HSD-1 inhibits insulin secretion from isolated murine pancreatic beta cells. (See, B. Davani et al., J. Biol. Chem., 275: 34841-34844, 2000). Incubation of isolated islets with an 11β-HSD-1 inhibitor improves glucose stimulated insulin secretion. An earlier study suggested that glucocorticoids reduce insulin secretion in vivo. (B. Billaudel et al., Horm. Metab. Res. 11: 555-560, 1979). Therefore, inhibition of 11β-HSD-1 enzyme in the pancreas may improve glucose stimulated insulin release.

In clinical ophthalmology, one of the most significant complications caused by using topical and systemic glucocorticoids is corticosteroid-induced glaucoma. This condition is characterized by a significant increase in intraocular pressure (IOP). A recent study indicates that administration of a non-specific 11β-HSD-1 inhibitor, carbenoxolone, to healthy volunteers for seven days resulted in a 17% reduction of IOP. Therefore, administration of 11β-HSD-1 specific inhibitors could be used for the treatment of glaucoma.

In certain disease states, such as tuberculosis, psoriasis, and stress in general, high glucocorticoid activity shifts the immune response to a humoral response, when in fact a cell based response may be more beneficial to the patients. Inhibition of 11β-HSD-1 activity may reduce glucocorticoid levels, thereby shifting the immuno response to a cell based response. (D. Mason, Immunology Today, 12: 57-60, 1991, G. A. W. Rook, Baillier's Clin. Endocrinol. Metab. 13: 576-581, 1999). Therefore, administration of 11β-HSD-1 specific inhibitors could be used for the treatment of tuberculosis, psoriasis, stress in general, and diseases or conditions where high glucocorticoid activity shifts the immune response to a humoral response.

Excess glucocorticoids decrease bone mineral density and increases fracture risk. This effect is mainly mediated by inhibition of osteoblastic bone formation, which results in a net bone loss (C. H. Kim et al. J. Endocrinol. 162: 371-379, 1999, C. G. Bellows et al. 23: 119-125, 1998, M. S. Cooper et al., Bone 27: 375-381, 2000). Therefore, reduction of cortisol levels by administration of an 11β-HSD-1 specific inhibitor may be useful for preventing bone loss due to osteroporosis.

Therapeutic Compositions-Administration-Dose Ranges

Therapeutic compositions of the present compounds comprise an effective amount of the same formulated with one or more therapeutically suitable excipients. The term “therapeutically suitable excipient,” as used herein, generally refers to pharmaceutically suitable, solid, semi-solid or liquid fillers, diluents, encapsulating material, formulation auxiliary and the like. Examples of therapeutically suitable excipients include, but are not limited to, sugars, cellulose and derivatives thereof, oils, glycols, solutions, buffers, colorants, releasing agents, coating agents, sweetening agents, flavoring agents, perfuming agents, and the like. Such therapeutic compositions may be administered parenterally, intracistemally, orally, rectally, intraperitoneally or by other dosage forms known in the art.

Liquid dosage forms for oral administration include, but are not limited to, emulsions, microemulsions, solutions, suspensions, syrups, and elixirs. Liquid dosage forms may also contain diluents, solubilizing agents, emulsifying agents, inert diluents, wetting agents, emulsifiers, sweeteners, flavorants, perfuming agents and the like.

Injectable preparations include, but are not limited to, sterile, injectable, aqueous, oleaginous solutions, suspensions, emulsions and the like. Such preparations may also be formulated to include, but are not limited to, parenterally suitable diluents, dispersing agents, wetting agents, suspending agents and the like. Such injectable preparations may be sterilized by filtration through a bacterial-retaining filter. Such preparations may also be formulated with sterilizing agents that dissolve or disperse in the injectable media or other methods known in the art.

The absorption of the compounds of the present invention may be delayed using a liquid suspension of crystalline or amorphous material having poor water solubility. The rate of absorption of the compounds generally depends upon the rate of dissolution and crystallinity. Delayed absorption of a parenterally administered compound may also be accomplished by dissolving or suspending the compound in oil. Injectable depot dosage forms may also be prepared by microencapsulating the same in biodegradable polymers. The rate of drug release may also be controlled by adjusting the ratio of compound to polymer and the nature of the polymer employed. Depot injectable formulations may also prepared by encapsulating the compounds in liposomes or microemulsions compatible with body tissues.

Solid dosage forms for oral administration include, but are not limited to, capsules, tablets, gels, pills, powders, granules and the like. The drug compound is generally combined with at least one therapeutically suitable excipient, such as carriers, fillers, extenders, disintegrating agents, solution retarding agents, wetting agents, absorbents, lubricants and the like. Capsules, tablets, and pills may also contain buffering agents. Suppositories for rectal administration may be prepared by mixing the compounds with a suitable non-irritating excipient that is solid at ordinary temperature but fluid in the rectum.

The present drug compounds may also be microencapsulated with one or more excipients. Tablets, dragees, capsules, pills, and granules may also be prepared using coatings and shells, such as enteric and release or rate controlling polymeric and nonpolymeric materials. For example, the compounds may be mixed with one or more inert diluents. Tableting may further include lubricants and other processing aids. Similarly, capsules may contain opacifying agents that delay release of the compounds in the intestinal tract.

Transdermal patches have the added advantage of providing controlled delivery of the present compounds to the body. Such dosage forms are prepared by dissolving or dispensing the compounds in suitable medium. Absorption enhancers may also be used to increase the flux of the compounds across the skin. The rate of absorption may be controlled by employing a rate controlling membrane. The compounds may also be incorporated into a polymer matrix or gel.

For a given dosage form, disorders of the present invention may be treated, prophylatically treated, or have their onset delayed in a patient by administering to the patient a therapeutically effective amount of compound of the present invention in accordance with a suitable dosing regimen. In other words, a therapeutically effective amount of any one of compounds of formulas I thru IX is administered to a patient to treat and/or prophylatically treat disorders modulated by the 11-beta-hydroxysteroid dehydrogenase type 1 enzyme. The specific therapeutically effective dose level for a given patient population may depend upon a variety of factors including, but not limited to, the specific disorder being treated, the severity of the disorder; the activity of the compound, the specific composition or dosage form, age, body weight, general health, sex, diet of the patient, the time of administration, route of administration, rate of excretion, duration of the treatment, drugs used in combination, coincidental therapy and other factors known in the art.

The present invention also includes therapeutically suitable metabolites formed by in vivo biotransformation of any of the compounds of formula I thru IX. The term “therapeutically suitable metabolite”, as used herein, generally refers to a pharmaceutically active compound formed by the in vivo biotransformation of compounds of formula I thru IX. For example, pharmaceutically active metabolites include, but are not limited to, compounds made by adamantane hydroxylation or polyhydroxylation of any of the compounds of formulas I thru IX. A discussion of biotransformation is found in Goodman and Gilman's, The Pharmacological Basis of Therapeutics, seventh edition, MacMillan Publishing Company, New York, N.Y., (1985).

The total daily dose (single or multiple) of the drug compounds of the present invention necessary to effectively inhibit the action of 11-beta-hydroxysteroid dehydrogenase type 1 enzyme may range from about 0.01 mg/kg/day to about 50 mg/kg/day of body weight, and more preferably about 0.1 mg/kg/day to about 25 mg/kg/day of body weight. Treatment regimens generally include administering from about 10 mg to about 1000 mg of the compounds per day in single or multiple doses.

It is understood that the foregoing detailed description and accompanying examples are merely illustrative and are not to be taken as limitations upon the scope of the invention, which is defined solely by the appended claims and their equivalents. Various changes and modifications to the disclosed aspects will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

1. A compound according to formula (I), 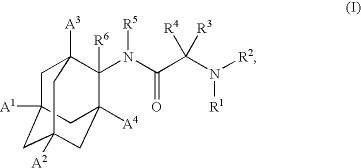
wherein
A¹, A², A³, and A⁴ are each a member independently selected from the group consisting of hydrogen, alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and, —C(R²³R²⁴)—N(R²⁵R²⁶);
n is 0 or 1;
p is 0 or 1;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and any intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle;
R³, R⁴ and any intervening atoms form a cycloalkyl; R³, R⁴ and any intervening carbon atoms form a non-aromatic heterocycle; and, R², R³ and any intervening carbon and nitrogen atoms form a non-aromatic heterocycle;
R⁵ is a member selected from the group consisting of hydrogen, alkyl, carboxyalkyl, carboxycycloalkyl, cycloalkyl, aryl, arylalkyl, aryloxyalkyl, heterocycle, heterocyclealkyl, and heterocycleoxyalkyl;
R⁶ is a member selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, and heterocycleoxyalkyl;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and any intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R¹¹, R¹² and any intervening atoms form a cycloalkyl; and, R¹¹, R¹² and any intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl; and, R¹⁵, R¹⁶ and any intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and any intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and any intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and any intervening atoms form a non-aromatic heterocycle;
provided that if R⁶ is hydrogen, then at least one of A¹, A², A³ and A⁴ is not hydrogen.

2. The compound according to claim 1, comprising a therapeutically suitable prodrug of the compound of formula (I).

3. The compound according to claim 1, comprising a therapeutically suitable salt of the compound of formula (I).

4. The compound according to claim 1, comprising a therapeutically suitable metabolite of the compound of formula (I).

5. A compound according to formula (II), 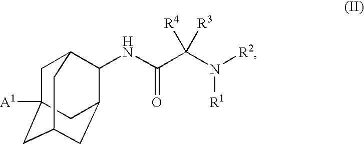
wherein,
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ, —C(O)—R¹⁰, —O—[C(R¹¹R¹²)]ₚ—C(O)—R¹³, —OR¹⁴, N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶);
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, aryl-heterocycle, and, R¹, R² and any intervening atoms form a heterocycle;
R³ and R⁴ are each a member independently selected from the group consisting of hydrogen, alkyl, carboxyalkyl, carboxycycloalkyl, cycloalkyl, haloalkyl, aryl, heterocycle;
R³, R⁴ and any intervening atoms form a cycloalkyl; R³, R⁴ and any intervening carbon atoms form a non-aromatic heterocycle; and, R², R³ and any intervening carbon and nitrogen atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl, and, R⁸, R⁹ and any intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and any intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and any intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and any intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and any intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and any intervening atoms form a non-aromatic heterocycle.

6. The compound according to claim 5, comprising a therapeutically suitable prodrug of the compound of formula (II).

7. The compound according to claim 5, comprising a therapeutically suitable salt of the compound of formula (II).

8. The compound according to claim 5, comprising a therapeutically suitable metabolite of the compound of formula (II).

9. The compound according to formula (III), 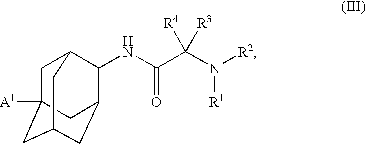
wherein
A¹ is a member selected from the group consisting of alkyl, alkyl-NH-alkyl, alkylcarbonyl, alkylsulfonyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R³, —OR¹⁴, —N(R¹⁵R¹⁶), —CO₂R¹⁷, —C(O)—N(R¹⁸R¹⁹), —C(R²⁰R²¹)—OR²², and, —C(R²³R²⁴)—N(R²⁵R²⁶;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle;
R³ and R⁴ are each a member independently selected from the group consisting of hydrogen, alkyl, carboxyalkyl, carboxycycloalkyl, cycloalkyl, aryl, and, heterocycle;
R⁷ is selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R⁸, R⁹ and any intervening atoms form a cycloalkyl; and, R⁸, R⁹ and any intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R¹¹, R¹² and any intervening atoms form a cycloalkyl; and, R¹¹, R¹² and any intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and any intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and any intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and any intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and any intervening atoms form a non-aromatic heterocycle.

10. The compound according to claim 9, comprising:
E-4-(2-Methyl-2-phenylamino-propionylamino)-adamantane-1-carboxylic acid amide.

11. The compound according to claim 9, comprising a therapeutically suitable prodrug of the compound of formula (III).

12. The compound according to claim 9, comprising a therapeutically suitable salt of the compound of formula (III).

13. The compound according to claim 9, comprising a therapeutically suitable metabolite of the compound of formula (III).

14. A compound according to formula (IV), 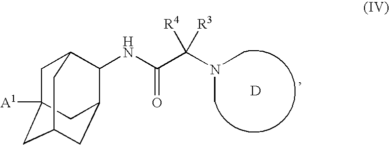
wherein,
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ, —C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and, —C(R²³R²⁴)—N(R²⁵R²⁶);
D is a non-aromatic heterocycle;
R³ and R⁴ are each a member independently selected from the group consisting of hydrogen, alkyl, carboxyalkyl, carboxycycloalkyl, cycloalkyl, haloalkyl, aryl, heterocycle;
R³, R⁴ and any intervening atoms form a cycloalkyl; and, R³, R⁴ and any intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R⁸, R⁹ and any intervening atoms form a cycloalkyl; and, R⁸, R⁹ and any intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R¹¹, R¹² and any intervening atoms form a cycloalkyl; and, R¹¹ and R¹² and any intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl; and, R¹⁵, R¹⁶ and any intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and any intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and any intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and any intervening atoms form a non-aromatic heterocycle.

15. The compound according to claim 14, comprising a member selected from the group consisting of:
2-[(cis)-2,6-dimethylmorpholin-4-yl]-N-[(E)-5-hydroxy-2-adamantyl]propanamide;
2-azepan-1-yl-N-[(E)-5-hydroxy-2-adamantyl]propanamide;
E-4-[2-(3,3-Difluoro-piperidin-1-yl)-acetylamino]-adamantane-1-carboxylic acid;
E-4-[2-(3,3-difluoro-piperidin-1-yl)-acetylamino]-adamantane-1-carboxylic acid amide;
E-4-[2-(3,3-difluoropiperidine-1-yl)-propionylamino]-adamantane-1-carboxylic acid amide;
E-4-[2-Methyl-2-(1,2,4,5-tetrahydro-benzo[d]azepin-3-yl)-propionylamino]-adamantane-1-carboxylic acid;
E-4-{2-[5-(6-Chloro-pyridin-3-yl)-hexahydro-pyrrolo[3,4-c]pyrrol-2-yl]-2-methyl-propionylamino}-adamantane-1-carboxylic acid amide;
E-4-{2-[4-(5-Fluoro-pyridin-3-yl)-[1,4]diazepan-1-yl]-2-methyl-propionylamino}-adamantane-1-carboxylic acid amide;
E-4-[2-methyl-2-(3-pyridin-3-yl-3,9-diazbicyclo[4.2.1]non-9-yl)-propionylamino]-adamantane-1-carboxylic acid amide;
E-4-[2-(3,3-difluoro-piperidin-1-yl)-2-methyl-propionylamino]-adamantane-1-carboxylic acid amide;
E-4-[2-(3,3-Difluoro-piperidin-1-yl)-2-methyl-propionylamino]-adamantane-1-carboxylic acid 3,4-dimethoxy-benzylamide;
E-4-[({4-[2-(3,3-Difluoro-piperidin-1-yl)-2-methyl-propionylamino]-adamantane-1-carbonyl}-amino)-methyl]-benzoic acid;
E-4-[2-(3,3-Difluoro-piperidin-1-yl)-2-methyl-propionylamino]-adamantane-1-carboxylic acid (furan-2-ylmethyl)-amide;
E-4-[2-(3,3-Difluoro-piperidin-1-yl)-2-methyl-propionylamino]-adamantane-1-carboxylic acid (thiazol-5-ylmethyl)-amide;
E-4-[2-(3,3-Difluoro-piperidin-1-yl)-2-methyl-propionylamino]-adamantane-1-carboxylic acid 2-methoxy-benzylamide;
E-4-[2-methyl-2-(3-pyridin-3-yl-3,9-diazbicyclo[4.2.1]non-9-yl)-propionylamino]-adamantane-1-carboxylic acid amide;
E-4-{2-methyl-2-[5-(3-trifluoromethyl-phenyl)-[1,5]diazocan-1-yl]-propionylamino}-adamantane-1-carboxylic acid; and
E-4-{2-[7-(5-bromo-pyridin-2-yl)-3,7-diazbicyclo[3.3.1]non-3-yl]-2-methyl-propionylamino}-adamantane-1-carboxylic acid amide.

16. The compound according to claim 14, comprising a therapeutically suitable prodrug of the compound of formula (IV).

17. The compound according to claim 14, comprising a therapeutically suitable salt of the compound of formula (IV).

18. The compound according to claim 14, comprising a therapeutically suitable metabolite of the compound of formula (IV).

19. A compound according to formula (V), 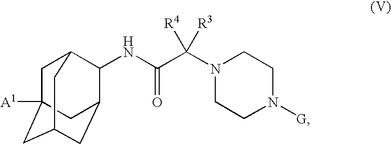
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶);
G is a member selected from the group consisting of aryl and heterocycle;
R³ and R⁴ are each a member independently selected from the group consisting of hydrogen, alkyl, carboxyalkyl, carboxycycloalkyl, cycloalkyl, haloalkyl, aryl, heterocycle;
R³, R⁴ and any intervening atoms form a cycloalkyl; and, R³, R⁴ and any intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R⁸, R⁹ and any intervening atoms form a cycloalkyl; and, R⁸, R⁹ and any intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R¹¹, R¹² and any intervening atoms form a cycloalkyl; and, R¹¹, R¹² and any intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and any intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and any intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and any intervening atoms form a non-aromatic heterocycle; and,
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and any intervening atoms form a non-aromatic heterocycle.

20. The compound according to claim 19, comprising a member selected from the group consisting of:
N-[(Z)-5-hydroxy-2-adamantyl]-2-{4-[5-(trifluoromethyl)pyridin-2-yl]piperazin-1-yl}acetamide;
N-[(E)-5-hydroxy-2-adamantyl]-2-{4-[5-(trifluoromethyl)pyridin-2-yl]piperazin-1-yl}acetamide;
N-[(E)-5-hydroxy-2-adamantyl]-2-{4-[5-(trifluoromethyl)pyridin-2-yl]piperazin-1-yl}propanamide;
(E)-4-[({4-[5-(trifluoromethyl)pyridin-2-yl]piperazin-1-yl}acetyl)amino]-1-adamantyl carbamate;
(E)-4-[(2-{4-[5-(trifluoromethyl)pyridin-2-yl]piperazin-1-yl}acetyl)amino]-1-adamantyl acetate;
N-[(E)-5-(acetylamino)-2-adamantyl]-2-{4-[5-(trifluoromethyl)pyridin-2-yl]piperazin-1-yl}acetamide;
N-[(E)-5-fluoro-2-adamantyl]-2-{4-[5-(trifluoromethyl)pyridin-2-yl]piperazin-1-yl}acetamide;
N-[(Z)-5-fluoro-2-adamantyl]-2-{4-[5-(trifluoromethyl)pyridin-2-yl]piperazin-1-yl}acetamide;
N-[(E)-5-hydroxy-2-adamantyl]-2-[4-(5-methylpyridin-2-yl)piperazin-1-yl]propanamide;
N-[(E)-5-hydroxy-2-adamantyl]-2-methyl-2-{4-[5-(trifluoromethyl)pyridin-2-yl]piperazin-1-yl}propanamide;
E-4-{2-Methyl-2-[4-(5-trifluoromethyl-pyridin-2-yl)-piperazin-1-yl]-propionylamino}-adamantane-1-carboxylic acid;
E-4-({1-[4-(5-Trifluoromethyl-pyridin-2-yl)-piperazin-1-yl]-cyclopropanecarbonyl}-amino)-adamantane-1-carboxylic acid;
E-4-({1-[4-(5-Trifluoromethyl-pyridin-2-yl)-piperazin-1-yl]-cyclopropanecarbonyl}-amino)-adamantane-1-carboxylic acid amide;
E-4-{2-[4-(5-trifluoromethyl-pyridin-2-yl)-piperazin-1-yl]-butyrylamino}-adamantane-1-carboxylic acid amide;
E-4-{2-Cyclopropyl-2-[4-(5-trifluoromethyl-pyridin-2-yl)-piperazin-1-yl]-acetylamino}-adamantane-1-carboxylic acid amide;
E-4-({1-[4-(5-Ttrifluoromethyl-pyridin-2-yl)-piperazin-1-yl]-cyclobutanecarbonyl}-amino)-adamantane-1-carboxylic acid amide;
E-N-(5-Hydroxymethyl-adamantan-2-yl)-2-[4-(5-trifluoromethyl-pyridin-2-yl)-piperazin-1-yl]-isobutyramide;
E-N-(5-Formyl-adamantan-2-yl)-2-[4-(5-trifluoromethyl-pyridin-2-yl)-piperazin-1-yl]-isobutyramide;
E-4-{2-methyl-2-[4-(5-trifluoromethyl-pyridin-2-yl)-piperazin-1-yl]-propionylamino}-adamantane-1-carboxylic acid amide;
E-4-{2-methyl-2-[4-(5-trifluoromethyl-pyridin-2-yl)-piperazin-1-yl]-propionylamino}-adamantane-1-carboxylic acid hydroxyamide;
E-4-{2-[4-(5-Trifluormethyl-pyridin-2-yl)-piperazin-1-yl]-acetylamino}-adamantane-1-carboxylic acid;
E-4-{2-[4-(5-trifluoromethyl-pyridin-2-yl)-piperazin-1-yl]-acetylamino}-adamantane-1-carboxylic acid amide;
E-4-{2-[4-(5-Chloro-pyridin-2-yl)-piperazin-1-yl]-2-methyl-propionylamino}-adamantane-1-carboxylic acid;
E-4-[2-Methyl-2-(4-m-tolyl-[1,4]diazepan-1-yl)-propionylamino]-adamantane-1-carboxylic acid; and
E-4-{2-[4-(5-Trifluormethyl-pyridin-2-yl)-piperazin-1-yl]-acetylamino}-adamantane-1-carboxamide.

21. The compound according to claim 19, comprising a therapeutically suitable prodrug of the compound of formula (V).

22. The compound according to claim 19, comprising a therapeutically suitable salt of the compound of formula (V).

23. The compound according to claim 19, comprising a therapeutically suitable metabolite of a compound of formula (V).

24. A compound of formula (VI), 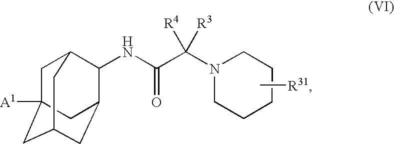
wherein
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R¹³, —OR¹⁴, —N(R¹⁵R¹⁶), —CO₂R¹⁷, —C(O)—N(R¹⁸R¹⁹), —C(R²⁰R²¹)—OR²², and —C(R²³R²⁴)—N(R²⁵R²⁶);
R³ and R⁴ are each a member independently selected from the group consisting of hydrogen, alkyl, carboxyalkyl, carboxycycloalkyl, cycloalkyl, haloalkyl, aryl, heterocycle;
R³, R⁴ and any intervening atoms form a cycloalkyl; and, R³, R⁴ and any intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R⁸ and R⁹ including any intervening atoms form a cycloalkyl; and, R⁸, R⁹ and any intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl, R¹¹ and R¹² including any intervening atoms form a cycloalkyl, and, R¹¹, R¹² and any intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰;
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl, and, R¹⁵, R¹⁶ and any intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²⁵ and R²⁶ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and any intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²¹ and any intervening atoms form a non-aromatic heterocycle;
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and any intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

25. The compound according to claim 24, comprising a member selected from the group consisting of:
N-[(Z)-5-hydroxy-2-adamantyl]-2-(4-hydroxypiperidin-1-yl)propanamide;
N-[(E)-5-hydroxy-2-adamantyl]-2-(4-hydroxypiperidin-1-yl)propanamide;
E-4-[2-Methyl-2-(4-phenyl-piperidin-1-yl)-propionylamino]-adamantane-1-carboxylic acid; and
E-4-{2-[4-(4-Chloro-phenyl)-piperidin-1-yl]-2-methyl-propionylamino}-adamantane-1-carboxylic acid.

26. The compound according to claim 23, comprising a therapeutically suitable prodrug of the compound of formula (VI).

27. The compound according to claim 23, comprising a therapeutically suitable salt of the compound of formula (VI).

28. The compound according to claim 23, comprising a therapeutically suitable metabolite of the compound of formula (VI).

29. A compound of formula (VII), 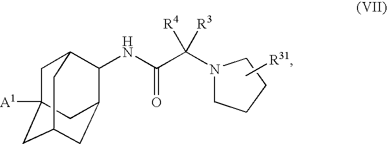
wherein,
A¹ is a member selected from the group consisting of alkyl, alkyl-NH-alkyl, alkylcarbonyl, alkylsulfonyl, cycloalkyl, cycloalkylcarbonyl, cycloalkylsulfonyl, arylcarbonyl, arylsulfonyl, heterocyclecarbonyl, heterocyclesulfonyl, aryl, arylalkyl, aryloxyalkyl, carboxyalkyl, carboxycycloalkyl, halogen, haloalkyl, heterocycle, heterocyclealkyl, heterocycleoxyalkyl, —NR⁷—[C(R⁸, R⁹)]ₙ—C(O)—R¹⁰, —O—[C(R¹¹R¹²)]ₚ—C(O)—R³, —OR⁴, —N(R¹⁵R⁶), —CO₂R¹⁷, —C(O)—N(R¹⁸R¹⁹), —C(R²⁰R²¹)—OR²², and —C(R²³R²⁴)—N(R²⁵R²⁶);
R³ and R⁴ are each a member independently selected from the group consisting of hydrogen, alkyl, carboxyalkyl, carboxycycloalkyl, cycloalkyl, haloalkyl, aryl, heterocycle;
R³, R⁴ and any intervening atoms form a cycloalkyl; and, R³, R⁴ and any intervening carbon atoms form a non-aromatic heterocycle;
R⁷ is a member selected from the group consisting of hydrogen, alkyl, carboxyalkyl, cycloalkyl, carboxycycloalkyl, aryl, arylalkyl, aryloxyalkyl, aryloxy, hydroxy, alkoxy, cycloalkyloxy, heterocycleoxy, heterocycle, heterocyclealkyl, and heterocycleoxyalkyl;
R⁸ and R⁹ are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R⁸, R⁹ and any intervening atoms form a cycloalkyl; and, R⁸, R⁹ and any intervening atoms form a non-aromatic heterocycle;
R¹⁰ is a member selected from the group consisting of hydrogen, alkyl, carboxy, carboxyalkyl, cycloalkyl, carboxycycloalkyl, aryl, aryloxy, arylalkyl, aryloxyalkyl, hydroxy, alkoxy, cycloalkyloxy, heterocycleoxy, heterocycle, heterocyclealkyl, heterocycleoxyalkyl, and —N(R²⁷R²⁸);
R¹¹ and R¹² are each a member independently selected from the group consisting of hydrogen, alkyl, carboxy, carboxyalkyl, cycloalkyl, carboxycycloalkyl, aryl, arylalkyl, aryloxyalkyl, heterocycle, heterocyclealkyl, heterocycleoxyalkyl; R¹¹, R¹² and any intervening atoms form a cycloalkyl; and, R¹¹, R¹² and any intervening atoms form a non-aromatic heterocycle;
R¹³ is selected from the group consisting of hydrogen, alkyl, carboxy, carboxyalkyl, cycloalkyl, carboxycycloalkyl, aryl, arylalkyl, aryloxy, aryloxyalkyl, hydroxy, alkoxy, cycloalkyloxy, heterocycleoxy, heterocycle, heterocyclealkyl, heterocycleoxyalkyl, and —N(R²⁹R³⁰);
R¹⁴ is a member selected from the group consisting of hydrogen, alkyl, carboxyalkyl, cycloalkyl, carboxycycloalkyl, aryl, arylalkyl, aryloxyalkyl, haloalkyl, heterocycle, heterocyclealkyl, and heterocycleoxyalkyl;
R¹⁵ and R¹⁶ are each a member independently selected from the group consisting of hydrogen, alkyl, carboxyalkyl, cycloalkyl, carboxycycloalkyl, aryl, arylalkyl, aryloxyalkyl, heterocycle, heterocyclealkyl, heterocycleoxyalkyl, alkylsufonyl, cycloalkylsulfonyl, arylsulfonyl, heterocyclesulfonyl; and, R⁵, R¹⁶ and any intervening atoms form a heterocycle;
R¹⁷ is a member selected from the group consisting of hydrogen, alkyl, carboxyalkyl, cycloalkyl, carboxycycloalkyl, aryl, arylalkyl, aryloxyalkyl, heterocycle, heterocyclealkyl, and heterocycleoxyalkyl;
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl; and, R¹⁸, R¹⁹ and any intervening atoms form a non-aromatic heterocycle;
R²⁰, R²¹ and R²² are each a member independently selected from the group consisting of hydrogen, alkyl, carboxyalkyl, carboxycycloalkyl, cycloalkyl, haloalkyl, aryl, and heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, cycloalkyl, aryl, and, heterocycle;
R²³ and R²⁴ are each a member independently selected from the group consisting of hydrogen, alkyl, alkylcarbonyl, alkylsulfonyl, carboxyalkyl, carboxycycloalkyl, cycloalkylcarbonyl, cycloalkylsulfonyl, arylcarbonyl, arylsulfonyl, heterocyclecarbonyl, heterocyclesulfonyl, hydroxy, alkoxy, cycloalkyloxy, aryloxy, heterocycleoxy, cycloalkyl, aryl, heterocycle, and, R²⁵, R²⁶ and any intervening atoms form a heterocycle;
R²⁷ and R²⁸ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁷, R²⁸ and any intervening atoms form a non-aromatic heterocycle;
R²⁹ and R³⁰ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R²⁹, R³⁰ and any intervening atoms form a non-aromatic heterocycle; and,
R³¹ is a member selected from the group consisting of alkyl, alkoxy, aryl, arylalkyl, aryloxy, aryloxyalkyl, cycloalkoxy, halogen, haloalkyl, heterocycle, heterocyclealkyl, heterocycleoxy, heterocycleoxyalkyl and hydroxy.

30. The compound according to claim 29, comprising a member selected from the group consisting of:
E-4-[2-(2-Trifluoromethyl-pyrrolidin-1-yl)-acetylamino]-adamantane-1-carboxylic acid;
E-4-[2-(2-trifluoromethyl-pyrrolidin-1-yl)-acetylamino]-adamantane-1-carboxylic acid amide;
E-4-[2-(3-fluoropyrrolidin-1-yl)-propionylamino]-adamantane-1-carboxylic acid amide;
E-4-[2-(2-trifluoromethylpyrrolidin-1-yl)-propionylamino]-adamantane-1-carboxylic acid amide;
E-4-[2-methyl-2-(2-trifluoromethyl-pyrrolidin-1-yl)-propionylamino]-adamantane-1-carboxylic acid amide; and
E-4-[2-(3-fluoro-pyrrolidin-1-yl)-2-methyl-propionylamino]-adamantane-1-carboxylic acid amide.

31. The compound according to claim 29, comprising a therapeutically suitable prodrug of the compound of formula (VII).

32. The compound according to claim 29, comprising a therapeutically suitable salt of the compound of formula (VII).

33. The compound according to claim 29, comprising a therapeutically suitable metabolite of the compound of formula (VII).

34. A compound according to formula (VIII) 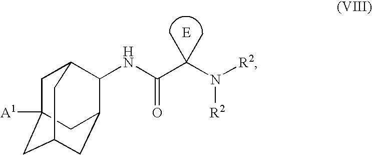
wherein
A¹ is a member selected from the group consisting of —OH, —CO₂H, carboxyalkyl, carboxycycloalkyl, and —C(O)—N(R¹⁸R¹⁹);
E is a member selected from the group consisting of cycloalkyl and non-aromatic heterocycle;
R¹ and R² are each a member independently selected from the group consisting of hydrogen, alkyl, alkoxyalkyl, alkyl-NH-alkyl, aryloxyalkyl, aryl-NH-alkyl, carboxyalkyl, carboxycycloalkyl, heterocycleoxyalkyl, heterocycle-NH-alkyl, cycloalkyl, aryl, arylalkyl, haloalkyl, heterocycle, heterocyclealkyl, heterocycle-heterocycle, and, aryl-heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle.

35. The compound according to claim 34, comprising a therapeutically suitable prodrug of the compound of formula (VIII).

36. The compound according to claim 34, comprising a therapeutically suitable salt of the compound of formula (VIII).

37. The compound according to claim 34, comprising a therapeutically suitable metabolite of the compound of formula (VIII).

38. A compound according to formula (IX), 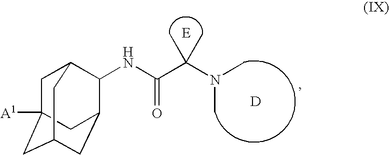
wherein
A¹ is a member selected from the group consisting of —OH, —CO₂H, carboxyalkyl, carboxycycloalkyl, and —C(O)—N(R¹⁸R¹⁹);
D is a non-aromatic heterocycle;
E is a member selected from the group consisting of cycloalkyl and non-aromatic heterocycle; and,
R¹⁸ and R¹⁹ are each a member independently selected from the group consisting of hydrogen, alkyl, carboxy, carboxyalkyl, cycloalkyl, cycloalkyloxy, carboxycycloalkyl, aryl, arylalkyl, aryloxy, aryloxyalkyl, heterocycle, heterocyclealkyl, heterocycleoxyalkyl, heterocycleoxy, hydroxy, alkoxy, alkylsufonyl, cycloalkylsulfonyl, arylsulfonyl, heterocyclesulfonyl, and, R¹⁸, R¹⁹ and any intervening atoms form a non-aromatic heterocycle.

39. The compound according to claim 38, comprising a therapeutically suitable prodrug of the compound of formula (IX).

40. The compound according to claim 38, comprising a therapeutically suitable salt of the compound of formula (IX).

41. The compound according to claim 38, comprising a therapeutically suitable metabolite of the compound of formula (IX).

42. A method of inhibiting 11-beta-hydroxysteroid dehydrogenase Type I enzyme, comprising administering to a mammal, a therapeutically effective amount of the compound of formula (I) of claim 1.

43. A method of inhibiting 11-beta-hydroxysteroid dehydrogenase Type I enzyme, comprising administering to a mammal, a therapeutically effective amount of the compound of formula (II) of claim 5.

44. A method of inhibiting 11-beta-hydroxysteroid dehydrogenase Type I enzyme, comprising administering to a mammal, a therapeutically effective amount of the compound of formula (III) of claim 9.

45. A method of inhibiting 11-beta-hydroxysteroid dehydrogenase Type I enzyme, comprising administering to a mammal, a therapeutically effective amount of the compound of formula (IV) of claim 14.

46. A method of inhibiting 11-beta-hydroxysteroid dehydrogenase Type I enzyme, comprising administering to a mammal, a therapeutically effective amount of the compound of formula (V) of claim 19.

47. A method of inhibiting 11-beta-hydroxysteroid dehydrogenase Type I enzyme, comprising administering to a mammal, a therapeutically effective amount of the compound of formula (VI) of claim 24.

48. A method of inhibiting 11-beta-hydroxysteroid dehydrogenase Type I enzyme comprising administering to a mammal, a therapeutically effective amount of a compound of formula (VII) of claim 29.

49. A method of inhibiting 11-beta-hydroxysteroid dehydrogenase Type I enzyme, comprising administering to a mammal, a therapeutically effective amount of the compound of formula (VIII) of claim 34.

50. A method of inhibiting 11-beta-hydroxysteroid dehydrogenase Type I enzyme, comprising administering to a mammal, a therapeutically effective amount of the compound of formula (IX) of claim 38.

51. A method of treating or prophylactically treating disorders in a mammal by inhibiting 11-beta-hydroxysteroid dehydrogenase Type I enzyme, comprising administering to a mammal, a therapeutically effective amount of the compound of formula (I) of claim 1.

52. A method of treating or prophylactically treating disorders in a mammal by inhibiting 11-beta-hydroxysteroid dehydrogenase Type I enzyme, comprising administering to a mammal, a therapeutically effective amount of the compound of formula (II) of claim 5.

53. A method of treating or prophylactically treating disorders in a mammal by inhibiting 11-beta-hydroxysteroid dehydrogenase Type I enzyme, comprising administering to a mammal, a therapeutically effective amount of the compound of formula (III) of claim 9.

54. A method of treating or prophylactically treating disorders in a mammal by inhibiting 11-beta-hydroxysteroid dehydrogenase Type I enzyme, comprising administering to a mammal, a therapeutically effective amount of the compound of formula (IV) of claim 14.

55. A method of treating or prophylactically treating disorders in a mammal by inhibiting 11-beta-hydroxysteroid dehydrogenase Type I enzyme, comprising administering to a mammal, a therapeutically effective amount of the compound of formula (V) of claim 19.

56. A method of treating or prophylactically treating disorders in a mammal by inhibiting 11-beta-hydroxysteroid dehydrogenase Type I enzyme, comprising administering to a mammal, a therapeutically effective amount of the compound of formula (VI) of claim 24.

57. A method of treating or prophylactically treating disorders in a mammal by inhibiting 11-beta-hydroxysteroid dehydrogenase Type I enzyme, comprising administering to a mammal, a therapeutically effective amount of the compound of formula (VII) of claim 29.

58. A method of treating or prophylactically treating disorders in a mammal by inhibiting 11-beta-hydroxysteroid dehydrogenase Type I enzyme, comprising administering to a mammal, a therapeutically effective amount of the compound of formula (VIII) of claim 34.

59. A method of treating or prophylactically treating disorders in a mammal by inhibiting 11-beta-hydroxysteroid dehydrogenase Type I enzyme, comprising administering to a mammal, a therapeutically effective amount of the compound of formula (IX) of claim 38.

60. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (I) of claim 1.

61. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (II) of claim 5.

62. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (III) of claim 9.

63. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (IV) of claim 14.

64. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (V) of claim 19.

65. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (VI) of claim 24.

66. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (VII) of claim 29.

67. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (VIII) of claim 34.

68. A method of treating or prophylactically treating non-insulin dependent type 2 diabetes in a mammal by inhibiting 11-beta-hydroxysteroid dehydrogenase Type I enzyme comprising administering to a mammal, a therapeutically effective amount of the compound of formula (IX) of claim 38.

69. A method of treating or prophylactically treating insulin resistance in a mammal by inhibiting 11-beta-hydroxysteroid dehydrogenase Type I enzyme comprising administering to a mammal, a therapeutically effective amount of the compound of formula (I) of claim 1.

70. A method of treating or prophylactically treating insulin resistance in a mammal by inhibiting 11-beta-hydroxysteroid dehydrogenase Type I enzyme comprising administering to a mammal, a therapeutically effective amount of the compound of formula (II) of claim 5.

71. A method of treating or prophylactically treating insulin resistance in a mammal by inhibiting 11-beta-hydroxysteroid dehydrogenase Type I enzyme comprising administering to a mammal, a therapeutically effective amount of the compound of formula (III) of claim 9.

72. A method of treating or prophylactically treating insulin resistance in a mammal by inhibiting 11-beta-hydroxysteroid dehydrogenase Type I enzyme comprising administering to a mammal, a therapeutically effective amount of the compound of formula (IV).

73. A method of treating or prophylactically treating insulin resistance in a mammal by inhibiting 11-beta-hydroxysteroid dehydrogenase Type I enzyme comprising administering to a mammal, a therapeutically effective amount of the compound of formula (V) of claim 19.

74. A method of treating or prophylactically treating insulin resistance in a mammal by inhibiting 11-beta-hydroxysteroid dehydrogenase Type I enzyme comprising administering to a mammal, a therapeutically effective amount of the compound of formula (VI) of claim 24.

75. A method of treating or prophylactically treating insulin resistance in a mammal by inhibiting 11-beta-hydroxysteroid dehydrogenase Type I enzyme comprising administering to a mammal, a therapeutically effective amount of the compound of formula (VII) of claim 29.

76. A method of treating or prophylactically treating insulin resistance in a mammal by inhibiting 11-beta-hydroxysteroid dehydrogenase Type I enzyme comprising administering to a mammal, a therapeutically effective amount of the compound of formula (VIII) of claim 34.

77. A method of treating or prophylactically treating insulin resistance in a mammal by inhibiting 11-beta-hydroxysteroid dehydrogenase Type I enzyme comprising administering to a mammal, a therapeutically effective amount of the compound of formula (IX) of claim 38.

78. A method of treating or prophylactically treating obesity in a mammal by inhibiting 11-beta-hydroxysteroid dehydrogenase Type I enzyme comprising administering to a mammal, a therapeutically effective amount of the compound of formula (I) of claim 1.

79. A method of treating or prophylactically treating obesity in a mammal by inhibiting 11-beta-hydroxysteroid dehydrogenase Type I enzyme comprising administering to a mammal, a therapeutically effective amount of the compound of formula (II) of claim 5.

80. A method of treating or prophylactically treating obesity in a mammal by inhibiting 11-beta-hydroxysteroid dehydrogenase Type I enzyme comprising administering to a mammal, a therapeutically effective amount of the compound of formula (III) of claim 9.

81. A method of treating or prophylactically treating obesity in a mammal by inhibiting 11-beta-hydroxysteroid dehydrogenase Type I enzyme comprising administering to a mammal, a therapeutically effective amount of the compound of formula (IV) of claim 14.

82. A method of treating or prophylactically treating obesity in a mammal by inhibiting 11-beta-hydroxysteroid dehydrogenase Type I enzyme comprising administering to a mammal, a therapeutically effective amount of the compound of formula (V) of claim 19.

83. A method of treating or prophylactically treating obesity in a mammal by inhibiting 11-beta-hydroxysteroid dehydrogenase Type I enzyme comprising administering to a mammal, a therapeutically effective amount of the compound of formula (VI) of claim 24.

84. A method of treating or prophylactically treating obesity in a mammal by inhibiting 11-beta-hydroxysteroid dehydrogenase Type I enzyme comprising administering to a mammal, a therapeutically effective amount of the compound of formula (VII) of claim 29.

85. A method of treating or prophylactically treating obesity in a mammal by inhibiting 11-beta-hydroxysteroid dehydrogenase Type I enzyme comprising administering to a mammal, a therapeutically effective amount of the compound of formula (VIII) of claim 34.

86. A method of treating or prophylactically treating obesity in a mammal by inhibiting 11-beta-hydroxysteroid dehydrogenase Type I enzyme comprising administering to a mammal, a therapeutically effective amount of the compound of formula (IX) of claim 38.

87. A method of treating or prophylactically treating lipid disorders in a mammal by inhibiting 11-beta-hydroxysteroid dehydrogenase Type I enzyme comprising administering to a mammal, a therapeutically effective amount of the compound of formula (I) of claim 1.

88. A method of treating or prophylactically treating lipid disorders in a mammal by inhibiting 11-beta-hydroxysteroid dehydrogenase Type I enzyme comprising administering to a mammal, a therapeutically effective amount of the compound of formula (II) of claim 5.

89. A method of treating or prophylactically treating lipid disorders in a mammal by inhibiting 11-beta-hydroxysteroid dehydrogenase Type I enzyme comprising administering to a mammal, a therapeutically effective amount of the compound of formula (III) of claim 9.

90. A method of treating or prophylactically treating lipid disorders in a mammal by inhibiting 11-beta-hydroxysteroid dehydrogenase Type I enzyme comprising administering to a mammal, a therapeutically effective amount of the compound of formula (IV) of claim 14.

91. A method of treating or prophylactically treating lipid disorders in a mammal by inhibiting 11-beta-hydroxysteroid dehydrogenase Type I enzyme comprising administering to a mammal, a therapeutically effective amount of the compound of formula (V) of claim 19.

92. A method of treating or prophylactically treating lipid disorders in a mammal by inhibiting 11-beta-hydroxysteroid dehydrogenase Type I enzyme comprising administering to a mammal, a therapeutically effective amount of the compound of formula (VI) of claim 24.

93. A method of treating or prophylactically treating lipid disorders in a mammal by inhibiting 11-beta-hydroxysteroid dehydrogenase Type I enzyme comprising administering to a mammal, a therapeutically effective amount of the compound of formula (VII) of claim 29.

94. A method of treating or prophylactically treating lipid disorders in a mammal by inhibiting 11-beta-hydroxysteroid dehydrogenase Type I enzyme comprising administering to a mammal, a therapeutically effective amount of the compound of formula (VIII) of claim 34.

95. A method of treating or prophylactically treating lipid disorders in a mammal by inhibiting 11-beta-hydroxysteroid dehydrogenase Type I enzyme comprising administering to a mammal, a therapeutically effective amount of the compound of formula (IX) of claim 38.

96. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (I) of claim 1.

97. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (II) of claim 5.

98. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (III) of claim 9.

99. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (IV) of claim 14.

100. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (V) of claim 19.

101. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (VI) of claim 24.

102. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (VII) of claim 29.

103. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (VIII) of claim 34.

104. A method of treating or prophylactically treating metabolic syndrome in a mammal by inhibiting 11-beta-hydroxysteroid dehydrogenase Type I enzyme comprising administering to a mammal, a therapeutically effective amount of the compound of formula (IX) of claim 38.

105. A method of treating or prophylactically treating diseases and conditions that are mediated by excessive glucocorticoid action in a mammal by inhibiting 11-beta-hydroxysteroid dehydrogenase Type I enzyme comprising administering to a mammal, a therapeutically effective amount of the compound of formula (I) of claim 1.

106. A method of treating or prophylactically treating a disease or condition mediated by excessive glucocorticoid action in a mammal by inhibiting 11-beta-hydroxysteroid dehydrogenase Type I enzyme comprising administering to a mammal, a therapeutically effective amount of the compound of formula (II) of claim 5.

107. A method of treating or prophylactically treating a disease or condition mediated by excessive glucocorticoid action in a mammal by inhibiting 11-beta-hydroxysteroid dehydrogenase Type I enzyme comprising administering to a mammal, a therapeutically effective amount of the compound of formula (III) of claim 9.

108. A method of treating or prophylactically treating a disease or condition mediated by excessive glucocorticoid action in a mammal by inhibiting 11-beta-hydroxysteroid dehydrogenase Type I enzyme comprising administering to a mammal, a therapeutically effective amount of the compound of formula (IV) of claim 14.

109. A method of treating or prophylactically treating a disease or condition mediated by excessive glucocorticoid action in a mammal by inhibiting 11-beta-hydroxysteroid dehydrogenase Type I enzyme comprising administering to a mammal, a therapeutically effective amount of the compound of formula (V) of claim 19.

110. A method of treating or prophylactically treating a disease and condition mediated by excessive glucocorticoid action in a mammal by inhibiting 11-beta-hydroxysteroid dehydrogenase Type I enzyme comprising administering to a mammal, a therapeutically effective amount of the compound of formula (VI) of claim 24.

111. A method of treating or prophylactically treating a disease or condition mediated by excessive glucocorticoid action in a mammal by inhibiting 11-beta-hydroxysteroid dehydrogenase Type I enzyme comprising administering to a mammal, a therapeutically effective amount of the compound of formula (VII) of claim 29.

112. A method of treating or prophylactically treating a disease or condition mediated by excessive glucocorticoid action in a mammal by inhibiting 11-beta-hydroxysteroid dehydrogenase Type I enzyme comprising administering to a mammal, a therapeutically effective amount of the compound of formula (VIII) of claim 34.

113. A method of treating or prophylactically treating a disease or condition mediated by excessive glucocorticoid action in a mammal by inhibiting 11-beta-hydroxysteroid dehydrogenase Type I enzyme comprising administering to a mammal, a therapeutically effective amount of the compound of formula (IX) of claim 38.

114. A pharmaceutical composition comprising a therapeutically effective amount of the compound of formula (I) of claim 1 in combination with a pharmaceutically suitable carrier.

115. A pharmaceutical composition comprising a therapeutically effective amount of the compound of formula (II) of claim 5 in combination with a pharmaceutically suitable carrier.

116. A pharmaceutical composition comprising a therapeutically effective amount of the compound of formula (III) of claim 9 in combination with a pharmaceutically suitable carrier.

117. A pharmaceutical composition comprising a therapeutically effective amount of the compound of formula (IV) of claim 14 in combination with a pharmaceutically suitable carrier.

118. A pharmaceutical composition comprising a therapeutically effective amount of the compound of formula (V) of claim 19 in combination with a pharmaceutically suitable carrier.

119. A pharmaceutical composition comprising a therapeutically effective amount of the compound of formula (VI) of claim 24 in combination with a pharmaceutically suitable carrier.

120. A pharmaceutical composition comprising a therapeutically effective amount of the compound of formula (VII) of claim 29 in combination with a pharmaceutically suitable carrier.

121. A pharmaceutical composition comprising a therapeutically effective amount of the compound of formula (VIII) of claim 34 in combination with a pharmaceutically suitable carrier.

122. A pharmaceutical composition comprising a therapeutically effective amount of the compound of formula (IX) of claim 38 in combination with a pharmaceutically suitable carrier.
